(19) United States
(12) Patent Application Publication
(10) Pub. No.: US 2015/0313961 A1
(43) Pub. Date: Nov. 5, 2015

(54) Materials and Methods for Regulating Whole Body Glucose Homeostasis

(71) Applicant: Indiana University Research & Technology Corporation, Indianapolis, IN (US)

(72) Inventors: Debbie C. Thurmond, Indianapolis, IN (US); Raghavendra Mirmira, Zionsville, IN (US)

(73) Assignee: Indiana University Research & Technology Corporation, Indianapolis, IN (US)

(21) Appl. No.: 14/626,809

(22) Filed: Feb. 19, 2015

Related U.S. Application Data
(60) Provisional application No. 61/986,509, filed on Apr. 30, 2014.
(60) Provisional application No. 62/087,379, filed on Dec. 4, 2014.

(51) Int. Cl. A61K 38/17; C07K 14/47; A61N 7/00; A61K 35/39; A61N 1/32; A61M 37/00; C07K 14/705; A61K 48/00

(52) U.S. Cl. CPC A61K 38/177; C07K 14/705; C07K 14/4705; C07K 14/47; A61K 48/0058; A61K 48/0025; A61K 38/1709; A61K 35/39; A61N 1/327; A61M 37/0092; A61N 7/00

(57) ABSTRACT

Described herein are methods and materials for improving whole body glucose homeostasis in a subject. The methods and materials described herein are useful for preventing, delaying, and/or treating insulin-related diseases and conditions, including but not limited to type I and II diabetes, chronic pancreatitis, pancreatectomy, insulin resistance, prediabetes, and age-related insulin resistance, by increasing the efficiency of insulin secretion by β cells, and increasing the sensitivity of peripheral tissues, including skeletal muscle and adipose tissues.

### CROSS-REFERENCE TO RELATED APPLICATIONS

This application claims the benefit of U.S. Provisional Application No. 61/986,509, filed Apr. 30, 2014, and U.S. Provisional Application No. 62/087,379, filed Dec. 4, 2014, the entire disclosures of which are expressly incorporated herein by reference for all purposes.

### STATEMENT REGARDING FEDERALLY SPONSORED RESEARCH

This invention was made with government support under DK067912, DK076614, DK083583 and DK060581 awarded by National Institutes of Health. The Government has certain rights in the invention.

### SEQUENCE LISTING

The instant application contains a Sequence Listing which has been submitted via EFS-web and is hereby incorporated by reference in its entirety. The ASCII copy, created on xxxx, xx, is named ******************, and is xxxxx bytes in size.

| SEQ. | | |
|---|---|---|
| ID NO: | Description | Sequence |
| 1 | Human | AF026007.1 |
| | Syntaxin 4 | |
| 2 | Mouse | NM009294.3 |
| | Syntaxin 4 | |
| 3 | Human | AF026007.1, with a |
| | Syntaxin 4 LE | nucleic acid mutation |
| | | resulting in the amino |
| | | acid mutation of |
| | | L173A/E174A |
| 4 | Mouse | NM009294.3, with a |
| | Syntaxin 4 LE | nucleic acid mutation |
| | | resulting in the amino |
| | | acid mutation of |
| | | L173A/E174A |
| 5 | Human Doc2b | NM003585.4 |
| 6 | Mouse Doc2b | NM007873 |
| 7 | Human Munc18c | NM007269.2 |
| 8 | Mouse Muc18c | NM011504 |
| 9 | Tg Doc2B | ggcagaggacaagtccctgg |
| | Primer #3 | |
| 10 | Tg Doc2B | agaggattgagcgttggcac |
| | Primer #9 | |
| 11 | Tg Doc2B | acgacgaggcttgcaggatcataa |
| | Primer #10 | |
| 12 | Wt Doc2B | ggaaagaaggcgaatggaag |
| | Primer O | |
| 13 | Wt Doc2B | tcactccagggttttcatcc |
| | Primer F | |

### BACKGROUND OF THE INVENTION

The regulation of glucose metabolism is a complex physiological process involving the interaction of several hormones, each of which themselves act in many target hormones. Among the hormones involved in glucose metabolism and homeostasis, insulin has the broadest array of functions. Once released by pancreatic β cells, insulin favors glucose uptake in whit adipose tissue and muscle, and suppresses gluconeogenesis in liver. The end result is to decrease blood glucose levels.

Several diseases and disorders or condition are associated with aberrant insulin secretion or signaling, including type 1 diabetes, type 2 diabetes, chronic pancreatitis, pancreatectomy, insulin resistance, prediabetes, and age-related insulin resistance.

Diabetes mellitus describes a metabolic disorder characterized by chronic hyperglycemia with disturbances of carbohydrate, fat and protein metabolism that result from defects in insulin secretion, insulin action, or both. The effects of diabetes include long-term damage, dysfunction and failure of various organs. In its most severe forms, ketoacidosis or a non-ketotic hyperosmolar state may develop and lead to stupor, coma and, in the absence of effective treatment, death. Often symptoms are not severe, not recognized, or may be absent. Consequently, hyperglycemia sufficient to cause pathological and functional changes may be present for a long time, occasionally up to ten years, before a diagnosis is made, usually by the detection of high levels of glucose in urine after overnight fasting during a routine medical work-up. The long-term effects of diabetes include progressive development of complications such as retinopathy with potential blindness, nephropathy that may lead to renal failure, neuropathy, microvascular changes, and autonomic dysfunction. People with diabetes are also at increased risk of cardiovascular, peripheral vascular, and cerebrovascular disease. There is also an increased risk of cancer.

Several pathogenetic processes are involved in the development of diabetes. These include processes which destroy the insulin-secreting beta cells of the pancreas with consequent insulin deficiency, and changes in liver and smooth muscle cells that result in the resistance to insulin uptake. The abnormalities of carbohydrate, fat and protein metabolism are due to deficient action of insulin on target tissues resulting from insensitivity to insulin or lack of insulin.

Regardless of the underlying cause, diabetes is subdivided into type 1 diabetes and type 2 diabetes. Type 1 diabetes results from autoimmune mediated destruction of the beta cells of the pancreas. Individuals with type 1 diabetes often become dependent on insulin for survival and are at risk for ketoacidosis. Patients with type 1 diabetes exhibit little or no insulin secretion, as manifested by low or undetectable levels of plasma C-peptide.

Treatment of type 1 diabetes at present is not satisfactory and the disease leads to serious life-threatening complications that can be only partly overcome with adequate control of insulin levels, which is usually difficult to accomplish in patients with juvenile onset.

Type 2 diabetes is the most common form of diabetes, and is characterized by disorders of insulin action and insulin secretion, either of which may be the predominant feature. Both are usually present at the time that this form of diabetes is clinically manifested. Type 2 diabetes patients are characterized with a relative, rather than absolute, insulin deficiency and are resistant to the action of insulin. At least initially, and often throughout their lifetime, these individuals do not need insulin treatment to survive. Type 2 diabetes accounts for 90-95% of all cases of diabetes. Whereas patients with this form of diabetes may have insulin levels that appear normal or elevated, the high blood glucose levels in these diabetic patients would be expected to result in even higher insulin values had their beta cell function been normal. Thus, insulin secretion is often defective and insufficient to compensate for the insulin resistance. On the other hand, some hyperglycemic individuals have essentially normal insulin action, but markedly impaired insulin secretion. Regulation of glucose homeostasis in the bloodstream must be tightly controlled to maintain healthy metabolic function. Low serum glucose levels (hypoglycemia) can lead to weakness, headaches, confusion, and if unchecked, ultimately convulsions, coma, and death.

Chronic pancreatitis is an inflammatory disease that causes structural and functional damage to functioning glandular tissue pancreas. This damage results in exocrine and endocrine defects. In particular, the lack of pancreatic enzymes interferes with the ability to properly digest fat. The production of insulin is also affected, which can lead to diabetes. Currently, there is a lack of effective preventative and therapeutic strategies for chronic pancreatitis.

The prevalence of type 2 diabetes mellitus (T2D) increases substantially with age. Both aging and T2D are accompanied by deleterious physiological changes, such as the development of insulin resistance, diminished glucose tolerance, fat accumulation and loss of muscle mass.

Therapies for type 1 and type 2 diabetes vary greatly, but in both cases exogenous insulin administration is often the primary therapeutic approach. However, constant administration of insulin is costly and cumbersome. Insulin administration typically requires frequent assessment of blood sugar to determine the dosage and administration schedule, and the insulin itself is typically administered by injection or infusion.

It would be beneficial to improve insulin secretion and/or insulin sensitivity in diabetic patients, in patients suffering from conditions such as chronic pancreatitis and prediabetes, both of which may lead to diabetes, and in aging patients, where the prevalence of T2D increases with age. By increasing insulin secretion and/or insulin sensitivity in these patients, whole body glucose homeostasis would be improved, thereby preventing onset or progression of, or treating, the patient's insulin-associated disease, disorder, or condition

### SUMMARY OF THE INVENTION

Described herein are methods and materials for improving whole body glucose homeostasis in a subject. The methods and materials described herein are useful for preventing, delaying, and/or treating insulin-related diseases and conditions, including but not limited to type I and II diabetes, chronic pancreatitis, pancreatectomy, insulin resistance, prediabetes, and age-related insulin resistance, by increasing the efficiency of insulin secretion by β cells, and increasing the sensitivity of peripheral tissues, including skeletal muscle and adipose tissues.

In a particular embodiment described herein is a method for improving whole body glucose homeostasis in a subject, comprising inducing overexpression of at least one gene in the subject, wherein the at least one gene is selected from the group consisting of: Syntaxin 4; and Doc2b, thereby improving whole body glucose homeostasis. In certain embodiments the method further comprises inducing overexpression of the Munc18c gene in the subject.

The subject is selected from a group consisting of: human; canine; rodent; primate; swine; equine; sheep; and feline. In a particular embodiment, the subject is human.

In one embodiment, the method for improving whole body glucose homeostasis in a subject is performed in a subject suffering from an insulin-related disease or condition selected from the group consisting of: type 1 diabetes; type 2 diabetes; chronic pancreatitis; pancreatectomy; insulin resistance; prediabetes; and age-related insulin resistance.

In other embodiments described herein, overexpression of the at least one gene in the subject is induced by at least one method selected from the group consisting of: cell therapy; and gene therapy.

Wherein overexpression of the at least one gene in the subject is induced by cell therapy, the cell therapy comprises administering to the subject cells overexpressing the at least one gene. In certain embodiments, overexpression of the at least one gene in the cells is achieved by introducing into the cells at least one nucleic acid having a sequence identity at least 80% identical to at least one nucleic acid selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; and SEQ ID NO: 8.

In other embodiments, the at least nucleic acid has a sequence identity selected from the group consisting of: at least 80%; at least 85%; at least 90%; at least 95%; at least 96%; at least 97%; at least 98%; at least 99%; and 100% identical with the at least one selected nucleic acid.

In yet another embodiment described herein, the introduction of the at least one nucleic acid is by transduction via viral vector. The viral vector can be selected from the group of viral vectors consisting of: a retroviral vector; adenovirus; herpes simplex virus; lentivirus; poxvirus; adeno-associated virus; and recombinant adeno-associated virus (rAAV). In one embodiment, the viral vector is recombinant adeno-associated virus (rAAV).

In one embodiment described herein, the nucleic acid is operably linked to a promoter.

In another embodiment described herein, the cells are autogenic, allogenic, or xenogenic. The cells are selected from the group of cells consisting of β cells; differentiated stem cells; undifferentiated stem cells; precursor cells, and reprogrammed insulin producing cells.

In yet another embodiment described herein, the cells are targeted to a particular tissue selected from the group consisting of: pancreatic tissue; skeletal muscle tissue; adipose tissue; brain tissue; heart tissue; liver tissue; spleen tissue; kidney tissue; and lung tissue.

The cells can be administered to the subject by a method of administration selected from the group consisting of: intravenous administration; and transplantation. In embodiments wherein the cells are administered by transplantation, cells are transplanted in at least one tissue selected from the group consisting of: pancreatic tissue; skeletal muscle tissue; adipose tissue; brain tissue; heart tissue; liver tissue; spleen tissue; kidney tissue; and lung tissue.

In one embodiment described herein, the cells are β cells overexpressing Syntaxin 4, and wherein the cells are transplanted in a tissue of the subject. In a particular embodiment, the tissue is kidney tissue.

In another embodiment described herein, the cells are autologous β cells isolated from the subject, transduced to induce overexpression of Syntaxin 4, and transplanted back into the subject.

Wherein overexpression of the at least one gene in the subject is induced by gene therapy, the gene therapy comprises administering to the subject at least one nucleic acid associated with the at least one gene. In an embodiment described herein, overexpression of the at least one gene is achieved by administering to the subject at least one nucleic acid having a sequence identity at least 80% identical to at least one nucleic acid selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; and SEQ ID NO: 8.

In another embodiment, the at least one nucleic acid has a sequence identity selected from the group consisting of: at least 80%; at least 85%; at least 90%; at least 95%; at least 96%; at least 97%; at least 98%; at least 99%; and 100% identical with the at least one selected nucleic acid.

In yet another embodiment, the at least one nucleic acid is present as a naked DNA, in a plasmid, or in a vector. Wherein the at least one nucleic acid is present in a vector, the vector is selected from a group of vectors consisting of: a retroviral vector; adenovirus; herpes simplex virus; lentivirus; poxvirus; adeno-associated virus; and recombinant adeno-associated virus (rAAV).

In certain embodiments described herein, the at least one nucleic acid is operably linked to a tissue specific promoter. In one embodiment, the promoter is β cell-specific.

The at least one nucleic acid can be administered to the subject by at least one method selected from the group consisting of: intravenous injection; direct organ or tissue injection; organ surface instillation; intra-arterial injection; intraportal injection; and retrograde intravenous injection. In some embodiments described herein, administration further comprises at least one physical method to enhance delivery of the nucleic acid selected from the group consisting of: electroporation; sonoporation; mechanical massage; and ultrasound exposure. In certain embodiments, the at least one physical method is applied to a target tissue. The target tissue is selected from the group consisting of: pancreatic tissue; skeletal muscle tissue; adipose tissue; brain tissue; heart tissue; liver tissue; spleen tissue; kidney tissue; and lung tissue.

In another embodiment described herein, the at least one nucleic acid is chemically modified. The chemical modification is selected from the group consisting of: lipoplex condensation and encapsulation; polymersome condensation and encapsulation; polyplex complex formation; dendrimer complex formation; inorganic nanoparticle complex formation; and cell penetrating peptide complex formation. In certain embodiments, the chemical modification further comprises the addition of a tissue-specific peptide.

In a particular embodiment described herein is a pharmaceutical composition comprising at least one nucleic acid associated with a gene selected from the group consisting of: Syntaxin 4; and Doc2b, wherein the at least one nucleic acid is operably linked to a promoter. The composition can further comprise a nucleic acid encoding Munc18c, wherein the nucleic acid encoding Munc18c is operably linked to a promoter. In certain embodiments, the promoter is specific for a tissue selected from the group consisting of: pancreatic tissue; skeletal muscle tissue; adipose tissue; brain tissue; heart tissue; liver tissue; spleen tissue; kidney tissue; and lung tissue.

In some embodiments described here, the pharmaceutical composition comprises at least one nucleic acid, wherein the at least one nucleic acid shares a sequence identity of at least 80% with at least one nucleic acid selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; and SEQ ID NO: 8. In other embodiments, the at least one nucleic acid shares a sequence identity selected from the group consisting of: at least 80%; at least 85%; at least 90%; at least 95%; at least 96%; at least 97%; at least 98%; at least 99%; and 100% with the at least one selected nucleic acid.

In another embodiment described herein, the at least one nucleic acid of the pharmaceutical composition is present in a vector. The vector can be selected from the group consisting of a retroviral vector; adenovirus; herpes simplex virus; lentivirus; poxvirus; adeno-associated virus; recombinant adeno-associated virus (rAAV); and naked plasmid DNA

In yet another embodiment described herein, the pharmaceutical composition further comprises a pharmaceutically acceptable excipient.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the Patent Office upon request and payment of the necessary fee.

FIG. 1A: Islet cells from type 2 diabetes (T2D) patients have reduced Syntaxin 4 (Syn4) compared to healthy individuals. The bar graph shows the average±S.E.; *P<0.05 versus healthy (non-diabetic) islet Syntaxin 4 levels. Detailed donor information for human islets is provided in Table 2.

FIG. 1B: Expression of Munc18-1, Syntaxin 1A, Syntaxin 4 and α-Tubulin by immunoblot in healthy compared to T2D subjects. Human islets obtained from five independent healthy donors and five T2D donors were lysed and Syntaxin 4 abundances were assessed by immunoblotting.

FIG. 1C: Human islet cells transduced with Syn4 have elevated glucose stimulated insulin secretion in an individual batch of human donor islets. Donor is a 25 year old Hispanic male with a BMI of 29.3.

FIG. 1D: Immunoblotting showing that the Syntaxin 4 transduced cells have increased Syntaxin 4 levels for the islet cells tested in FIG. 1C.

FIG. 1E: Human islet cells transduced with Syn4 have elevated glucose stimulated insulin secretion in an individual batch of human donor islets. Donor is a 59 year old Caucasian female with a BMI of 28.2.

FIG. 1F: Immunoblotting showing that the Syntaxin 4 transduced cells have increased Syntaxin 4 levels of the islet cells of FIG. 1E.

FIG. 1G: Adenovirus-Syn4 transduced islets have 80% and 130% more glucose stimulated insulin secretion as determined by an under the curve (AUC) analysis. Area under the curve (AUC) analysis was calculated for each donor islet phase of secretion and normalized to Ad-Control, shown as the average±S.E.; *P<0.05 versus Ad-Control.

FIG. 2A: Islet cells from human type 2 diabetes donors transduced with Ad-Syn4 have an improved response to stimulatory glucose. Human islets from a T2D donor was immediately transduced with control or Syntaxin 4 adenoviruses for subsequent perifusion analysis. Donor was a 54 year old, African female that had type 2 Diabetes for 5 years, a blood glucose of 170 mg/dL, BMI of 48.5.

FIG. 2B: Immunoblots showing Syntaxin 4 expression after transduction (relative to clathrin, as denoted below each blot) of the islet cells in FIG. 2A.

FIG. 2C: Islets from severe human type 2 diabetes donors had virtually no detectable insulin release, and that Syn4 cannot rescue insulin release in this case. Human islets from a T2D donor was immediately transduced with control or Syntaxin 4 adenoviruses for subsequent perifusion analysis. Donor was a 45 year old, Caucasian female, having type 2 diabetes for 10 years, blood glucose of 748 mg/dL, and a BMI of 28.5.

FIG. 2D: Immunoblots (IB) show Syntaxin 4 expression after transduction (relative to clathrin, as denoted below each blot) for the islet cells of FIG. 2C.

FIG. 2E: In vivo reversal of STZ-induced diabetic blood glucose levels in a minimal islet transplant model when provided with Syn4-enriched islet cells. SG mice were rendered diabetic using a single high-dose streptozotocin (STZ) intraperitoneal injection. Donor islets from Syntaxin 4 over-expressing transgenic (Tg) or littermate wild-type mice were transplanted into the diabetic NSG mice and random blood glucose readings captured at intervals shown over a 14 day period post-transplant. Raw data represent the outcomes of 9 transplant recipient mice of wild-type and 5 of Syntaxin 4 over-expressing donor islets. Data represent the average±S.E.; *P<0.05 versus wild-type islet recipients, and *P=0.055 versus wild-type.

FIG. 2F: Normalized data from in vivo studies from transplant model provided Syn4-enriched islet cells compared to wild type islets. Data represent the outcomes of 9 transplant recipient mice of wild-type and 5 of Syntaxin 4 over-expressing donor islets. Normalized (to the corresponding recipient blood glucose reading at Day 0 prior to transplantation) random (non-fasting) blood glucose was monitored before (day 0) and after transplantation (days 2, 6 and 14). Data represent the average±S.E.; *P<0.05 versus wild-type islet recipients.

FIG. 2G: AUC analysis of entire period of the in vivo transplant studies showing Syn4-enriched islet cells having a 25% improvement in blood glucose levels compared to wild type islet cells. Area under the curve (AUC) was calculated from panel C, where data represent the average±S.E.; *P<0.05 versus wild-type islet recipients.

FIG. 2H: Improvement of glycemia in transplantation models was attributable to Syn4 transduced islet cells. Three of the five Syntaxin 4 Tg islet transplant recipients underwent excision of the kidney (nephrectomy) to remove transplanted islets on day 15, and blood glucose levels assessed 3 days later; **P<0.05 versus before excision.

FIG. 2I: Pancreata from Syntaxin 4 Tg and littermate wild-type mice were evaluated for β cell area. Data are representative of 3 sets of pancreata.

FIG. 3A: Healthy (non-diabetic) human islets enriched with Syntaxin 4 exhibit amplified insulin release during both phases. Healthy human islets from a third donor were sized and sorted into two equal groups for immediate transduction with control or Syntaxin 4 adenoviruses (Ad-Control, Ad-Syntaxin 4) and subsequently perifused in parallel chambers with 2.8 mM glucose, stimulated with 20 mM glucose, and returned to 2.8 mM glucose to evaluate the regulated patterns of biphasic secretion. Donor was a 51 year old, Caucasian female with a BMI of 21.2.

FIG. 3B: Immunoblot detection (IB) of Syntaxin 4 expression levels in Ad-treated islets of FIG. 3A is shown, relative to the loading control (clathrin).

FIG. 4A: Perifusion traces of an additional batch of type 2 diabetic human islets enriched with Syntaxin 4. All islets were immediately transduced with control or Syntaxin 4 adenoviruses (Ad-Control, Ad-Syntaxin 4) upon receipt from the islet center and subsequently perifused in parallel chambers with 2.8 mM glucose, stimulated with 20 mM glucose, and returned to 2.8 mM glucose to evaluate the regulated patterns of biphasic secretion. Donor was a 43 year old male of unknown race, having T2DM for 10 years and a BMI of 37.0.

FIG. 4B: Perifusion traces of an additional batch of type 2 diabetic human islets enriched with Syntaxin 4. Donor is a 42 year old, Hispanic man with T2DM for 5 years and a BMI of 34.9.

FIG. 4C: Perifusion traces of an additional batch of type 2 diabetic human islets enriched with Syntaxin 4. Donor is a 58 year old, Caucasian man having T2DM for 5 years and a BMI of 48.9.

FIG. 4D: Perifusion traces of an additional batch of type 2 diabetic human islets enriched with Syntaxin 4. Donor is a 43 year old Hispanic of non-recorded gender having T2DM for 2 years, and HbA1c of 11.1%, and a BMI of 27.0.

FIG. 5: Transplantation of human islets enriched for Syntaxin 4. NOD-SCID mice were rendered diabetic using a single high-dose streptozotocin (STZ) intraperitoneal injection. Each batch of donor human islets was split into two groups for immediate transduction with Ad-Syntaxin 4 or Ad-Control particles. After 40 hr, transduced islets were transplanted under the renal capsule of recipient 8 week-old streptozotocin (STZ, 180 mg/kg) diabetic NOD-SCID mice and random blood glucose readings captured at intervals shown over a 16 day period post-transplant. Eight total grafts harboring human islets transduced with Ad-Syntaxin 4 (four grafts) or Ad-Control (four grafts) from two donors (male, middle aged, BMI's in the overweight/obese range, about 200 islets/graft) performed using identical methods, were combined as done previously (Levitt, et al. (2011). Diabetologia 54, 572-582). Statistical analysis was performed using Students two-tailed t-test; p=0.08 versus Ad-Syntaxin 4. AUC analysis: Ad-Control=7,540±640 (arbitrary units) vs. Ad-Syntaxin 4=5,649±589, p=0.07.

FIGS. 6A-6G: Protein expression in tissues of Doc2b transgenic mice. (A) Gastrocnemius skeletal muscle (Musc), Pancreas (Panc), and epigonadal fat were isolated from 3-5 pairs of Doc2b transgenic (Tg) (black bars) and wild-type (Wt) (white bars) littermate mice and immunoblotted (IB) for detection of SNARE and SNARE accessory proteins. (B) Doc2b abundances were normalized to Clathrin to account for minor variations in protein loading, *P<0.05 vs. Wild type. (C) Isolated islets were assessed for Doc2b and SNARE protein expression as described in panel A above, with (D) quantification of Doc2b abundances by AUC analysis. (E) Heart, liver and spleen were similarly assessed, as was (F) whole brain lysate, cerebellum (Cere) and hypothalamus (Hypo), for Doc2b levels; representative of results obtained from 3-5 pairs of mice. (G) GLUT4 protein abundance was assessed in heart, skeletal muscle and fat from mice in panel A. Data are representative of 3-5 pairs of mice.

FIGS. 7A-7C: Doc2b Tg mice have enhanced glucose tolerance. (A) Intraperitoneal glucose tolerance testing (IPGTT) of Doc2b Tg (black squares) and Wild type (white diamonds) littermate mice was performed in 4-6 month old female mice fasted for 6 hours. (B) Area under the curve (AUC) data are shown as the average±SE from 7 pair of mice; *P<0.05, vs. Wild type. (C) Insulin content present in serum taken prior to (Basal) and 10 min post-injection of glucose (Stimulated) during the IPGTT. Doc2b Tg (black bars) and Wild type (white bars). Data represent the average±SE from 6 pairs of mice; *P<0.05 vs. Wild type basal; †P<0.05 vs. Wild type glucose-stimulated.

FIGS. 8A-8D: Tetracycline-mediated repression of the DOC2B transgene reduces glucose tolerance to that of the Wild type mice. (A) Doc2b Tg (black squares) and Wild type (white diamonds) female mice assessed in FIG. 2 assays were subsequently administered tetracycline (1 mg/ml) in the drinking water for 1 week and IPGTT re-performed. (B) AUC analysis is shown as the average±SE from 7 pairs of mice. (C) Tissue extracts were immunoblotted and (D) quantified. Doc2b Tg (black bars) and Wild type (white bars). Data represent the average±SE of 3 independent sets of tissues.

FIGS. 9A-9C: Islets from Doc2b Tg mice exhibit potentiated biphasic insulin release. (A) Islets isolated from Doc2b Tg (black squares) and littermate Wild type mice (white diamonds) were perifused in parallel at 2.8 mmol/l glucose for 10 min, followed by 16.7 mmol/l glucose for 35 min, then returned to low glucose for 20 min. Eluted fractions were collected and insulin secretion determined by RIA, as depicted in this representative pair of traces. (B) AUC for first (11-17 min) and second (18-45 min) phases of insulin secretion was quantified from islets, normalized to baseline. Doc2b Tg (black bars) and Wild type (white bars). Data represent the average±SE of at 3 independent sets of perifused islets; *P<0.05 vs. Wild type (Wild type set equal to 1.0 and Tg normalized thereto for each phase per set). (C) Average insulin content per 10 islets from Doc2b Tg and Wild type littermate mice.

FIGS. 10A-10B: Doc2b Tg mice exhibit enhanced insulin sensitivity. (A) Insulin tolerance testing (ITT) of 7 pair of Doc2b Tg (black squares) and littermate Wild type female mice (white diamonds) fasted for 6 h. Data are shown as the mean percent of starting basal blood glucose concentrations±SE; *P<0.05 vs. Wild type mice. (B) Area over the curve (AOC) data are shown as the average±SE from 7 pairs of mice; *P<0.05, vs. Wild type.

FIGS. 11A-11D: Doc2b Tg mice show increased insulin-stimulated GLUT4 accumulation at the sarcolemma/transverse tubule plasma membranes of skeletal muscle. (A) GLUT4 abundance in the plasma membrane (PM) fractions was detected by immunoblot (left panel-Ponceau S shows protein loading). (B) Quantitation of GLUT4 accumulation in PM fractions is shown in the adjacent bar graph for 3 sets of mice; *P<0.05 vs. Wild type basal, †P<0.05 vs. Tg basal. (C) Whole skeletal muscle detergent homogenates from mice stimulated with insulin were immunoblotted for activated AKT (p-AKT^{S473}). Blots were stripped and reprobed for total AKT content. Data are representative of three independent sets of tissue homogenates. (D) PM fractions prepared from 3 sets of Doc2b Tg (black bars) and Wild type mice (white bars) from panel A were immunoblotted for Doc2b, Munc18c and Syn4. While Doc2b was significantly elevated in Tg vs. Wild type fractions (P<0.05), no statistical differences in Syn4 or Munc18c abundances were observed.

FIGS. 12A-12D: Over-expression of Doc2b coordinately decreases Munc18c-Syn4 binding while increasing Syn4 activation in L6 GLUT4-myc myoblasts. (A) Detergent lysates prepared from L6 GLUT4-myc myoblasts transfected to express GFP-tagged Doc2b or GFP alone and stimulated with insulin for 5 min were used in (anti-Syn4 immunoprecipitation reactions and co-precipitated Munc18c or Doc2b proteins detected by immunoblotting and (B) Munc18c/Syn4 percent quantified, or (C) GST-VAMP2 interaction assays for detection of the Syn4 present in lysates that is accessible to the exogenous GST-VAMP2 probe. Proteins were immunoblotted for Syn4, GST and GFP or GFP-Doc2b (left panel, about 75 kDa). (D) Quantitation is represented in the adjacent bar graphs as the average±SE of the ratio of Munc18c/Syn4 (%), and Syn4/GST-VAMP2 (fold), respectively, in 3 independent experiments each; *P<0.05 vs. GFP (right panel).

FIGS. 13A-13B: Body weight and food intake characteristics of the Doc2b Tg mice. (A) Body weights of 5 pairs of littermate Doc2b transgenic (black squares) and wild-type (white diamonds) female mice were monitored over a period of 9 weeks, and (B) food intake measured weekly for 7 weeks. Data represent the average±SE.

FIG. 14: IPGTT of the lower-expressing Doc2b Tg line. Doc2b Tg (black squares) and Wild type (white diamonds) 4-6 month old female littermate mice were fasted for 6 hours and IPGTT performed. Data represent the average±SE from 5 pairs of mice.

FIG. 15: Endogenous expression of Doc2b in detergent lysates prepared from L6 GLUT4-myc myoblasts transfected to express GFP-tagged Doc2b or GFP alone and stimulated with insulin. Data are representative of three independent experiments.

FIG. 16: Doc2b antibody specifically recognizes Doc2b. To confirm specificity of the antibody used, GFP-Doc2a and GFP-Doc2b were expressed in Chinese hamster ovary (CHO) cells by electroporation, and 48 h later detergent cell lysates were prepared and proteins resolved by 10% SDS-page for immunoblotting (IB). The full-length membrane is shown to reveal that the antibody recognized only one band of the expected molecular weight, no band was detected in Doc2a expressing cells. Anti GFP immunoblotting confirmed the expression and appropriate molecular weights of the fusion proteins.

FIGS. 17A-17H: Syn4 abundance and aging. Protein levels in pancreata of 4 and 18 months old C57BL/6J mice: Syn4 relative to tubulin (loading control) (A), phosphorylated mTOR relative to total mTOR (B). Data represent the mean±S.E. of 5 sets of pancreata. *P<0.05. (C) Proportion of all mice surviving, both sexes pooled, n=19 controls and 17 Syn4 Tg mice. (D-E) Survival curves for males (12 Con and 11 Syn4 Tg), versus females (7 Con and 6 Syn4 Tg). (F-H) Abundance/activation of proteins associated with aging in 3 paired sets of pancreata: phosphorylated/total mTOR, total SIRT1 and phosphorylated/total FoxO1 (pSer256).

FIGS. 18A-18J: Improved insulin and glucose tolerance in aged Syn4 Tg mice. Insulin tolerance tests were assessed in 25 month old mice with both sexes combined (A), or males alone (B); *P<0.05 (A, 8 Con and 12 Tg mice; B, 6 Con and 8 Tg mice). Glucose tolerance tests were performed in 24 month old mice, both with sexes combined (C), and in males alone (D) with bar graphs denoting area under the curve (AUC); *P<0.05 (C, 12 Con and 16 Tg; D, 9 Con and 11 Tg mice). Metabolic caging studies, wherein data represent the average±S.E. for 4-9 pairs of 12 month old male and female mice: body weights (E), food intake (F), respiratory quotient (G), and physical activity (H). DEXA scanning quantified body fat in 4 pairs of 8 month old male mice (I). Serum triglycerides (18 month old 7 Con and 7 Tg, sexes combined) (J); *p<0.05. BW, body weight.

FIGS. 19A-19G: Syn4 Tg mice show robust islet function. (A) Fasting serum insulin content in aged mice. Data represent the average±S.E. of 3 mice per group; *p<0.05 versus Con mice. (B-C) Serum insulin and glucagon contents during glucose tolerance testing in 6 month old male mice. Data represent the average±S.E. of 6-8 mice per group; *p<0.05 versus Con at same time point. Glucagon in serum from mice fasted (0) and after glucose stimulation (10 min). (D-E) Static insulin release from mouse islets ex vivo, and insulin content therefrom (n=3-4 islet batches from 6 month old mice); *p<0.05. (F-G) Islet cell distributions (red, glucagon stained α cell; green, insulin stained (cell) and percentage of β cell area in 18 month old mouse islets (n=3/group).

FIGS. 20A-20H: Protection of skeletal muscle and islet function in high-fat fed Syn4 Tg male mice. Body weight (A), epididymal fat pad weight (B), and food intake (C) in control (Con) and Syn4 Tg mice (n=8-12 per group). (D) Skeletal muscle GLUT4 accumulated at the cell surface (stimulation index=insulin/basal levels of GLUT4 at cell surface). Data represent the average±S.E. of 4 matched sets of mice/group; *p<0.05). Dashed line, average RD-Con mice. (E) Fasting serum insulin content during the HFD study (n=6 mice/group; *p<0.05). (F) Glucose stimulated insulin release (stimulation index=glucose stimulated/basal) of islets from mice fed HFD for 10 weeks (n=3 mice/group). Dashed line, average RD-Con mice; p=0.07, RD-Con versus HFDCon. (G) Islet insulin content after 10 weeks on HFD; data represent the average±S.E. of 6 mice/group; *p<0.05 versus Tg fed the RD. (H) β cell area for 4-6 month old mice fed HFD for 10 wks; n=3/group. Body weight (BW).

FIGS. 21A-21C: No differences in abundances of Syn4-based SNARE partners VAMP2, SNAP23 or Munc18c were observed with aging. Expression of proteins associated with aging in 3 paired sets of pancreata from 18 month old Syn4 Tg and Wt control mice were assessed by immunoblotting and quantified by optical scanning densitometry.

### DETAILED DESCRIPTION

Throughout this disclosure, various publications, patents and published patent specifications are referenced. The disclosures of these publications, patents and published patent specifications are hereby incorporated by reference into the present disclosure to more fully describe the state of the art to which this invention pertains.

Described herein are methods and materials for improving whole body glucose homeostasis in a subject. The methods and materials described herein are useful for preventing, delaying, and/or treating insulin-related diseases and conditions, including but not limited to type I and II diabetes, chronic pancreatitis, pancreatectomy, insulin resistance, prediabetes, and age-related insulin resistance, by increasing the efficiency of insulin secretion by β cells, and increasing the sensitivity of peripheral tissues, including skeletal muscle and adipose tissues.

### DEFINITIONS

As used herein, “subject” or “patient” refers an individual having impaired glucose homeostasis resulting from an insulin-related disease or condition including but not limited to type 1 diabetes, type 2 diabetes, chronic pancreatitis, pancreatectomy, insulin resistance, prediabetes, and age-related insulin resistance. A subject can be any individual animal suffering from an insulin-related disease or condition, including but not limited to human, canine, rodent, primate, swine, equine, sheep, and feline. “Insulin-related disease or condition” refers to any disease or condition characterized by an insulin deficiency or insulin resistance.

As used herein, the term “allogenic,” refers to tissue or cells from a distinct subject of the same species. The term “xenogenic” refers to tissue or cells from a subject of another species. The terms “autogenic” and “autologous” refer to tissue or cells derived from a subject, and re-introduced into the same subject.

As used herein, “effective amount” refers to an amount of a substance or composition sufficient to bring about improved whole body glucose. An effective amount, or effective dose, can be administered in one or more administrations. The precise determination of an effective amount will be affected by factors individual to each subject, including, but not limited to, the subject's age, size, type and extent of insulin-related disease or condition, method of administration, whether the substance or composition is administered alongside conventional therapy or on its own, and the desired result. It will be known to one of skill in the art how to determine an effective amount for a particular subject.

As used herein, a “promoter” means a nucleotide region comprising a nucleic acid (i.e., DNA) regulatory sequence, wherein the regulatory sequence is derived from a gene that is capable of binding RNA polymerase and initiating transcription of a downstream (3′-direction) coding sequence. Transcription promoters can include “inducible promoters” (where expression of a nucleic acid sequence operably linked to the promoter is induced by an analyte, cofactor, regulatory protein, etc.), “repressible promoters” (where expression of a polynucleotide sequence operably linked to the promoter is repressed by an analyte, cofactor, regulatory protein, etc.) and “constitutive promoters” (where expression of a polynucleotide sequence operably linked to the promoter is unregulated and therefore continuous). A “tissue specific” promoter is only active in a specific type or subset of tissues or cells. An “islet specific” promoter is only active in islet tissue or cells.

As used herein, “operably linked” means that elements of an expression sequence are configured so as to perform their usual function. Thus, control sequences (i.e., promoters) operably linked to a coding sequence are capable of effecting expression of the coding sequence. The control sequences need not be contiguous with the coding sequence, so long as they function to direct the expression thereof. Thus, for example, intervening untranslated, yet transcribed, sequences can be present between a promoter and a coding sequence, and the promoter sequence and still be considered “operably linked” to the coding sequence.

As used herein, the terms “providing,” “administering,” “introducing,” “delivering,” “placement,” and “transplanting” may used interchangeably herein to refer to the placement of a cell or nucleic acid described herein in a subject

“Pancreatectomy” refers to the surgical removal of the pancreas or a portion thereof.

As used herein, “pharmaceutically-acceptable excipient” means a pharmaceutically acceptable material, composition or vehicle involved in giving form or consistency to a pharmaceutical composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must be of sufficiently high purity to render it pharmaceutically-acceptable.

General Description

Insulin secretion and insulin action are critical for normal glucose homeostasis. Defects in either or both of these processes lead to Type 2 diabetes. Glucose-stimulated insulin secretion (GSIS) from islet beta cells, and insulin-stimulated glucose uptake into peripheral tissues, are events mediated by similar exocytotic mechanisms involving a family of Soluble N-Ethylmaleimide-Sensitive Factor Attachment Protein Receptor (SNARE) protein isoforms. SNARE-mediated exocytosis requires the association of two target membrane (t)-SNARE proteins (Syntaxins and SNAPs) with one vesicle/granule membrane (v)-SNARE protein (VAMP) to form a heterotrimeric SNARE core complex that promotes docking and fusion of vesicles/granules.

SNARE protein-mediated GSIS from islet beta cells is biphasic. The two glucose-stimulated phases are proposed to be supported by differing pools of VAMP2-localized granules mobilized to differing SNARE protein isoform binding sites at the cell surface, including Syntaxin 1A (Syn1A), Syntaxin 3, Syntaxin 4 (Syn4), and SNAP25 (or SNAP23). The v-SNARE protein VAMP8 also functions in insulin secretion, but is required selectively for GLP-1-enhanced GSIS. Similarly, insulin-stimulated glucose uptake into skeletal muscle and adipose cells involves the recruitment of VAMP2-bound vesicles containing the insulin-responsive glucose transporter 4 (GLUT4) to the cell surface, yet involves a smaller subset of SNARE isoforms at the cell surface: Syn4 and SNAP23.

Two isoforms of each type of t-SNARE participate in insulin secretion: SNAP23 and SNAP25, which appear to work interchangeably, and Syntaxin 1 and Syntaxin 4. Syntaxin 1 regulates only first-phase insulin secretion, and Syntaxin 4 facilitates both first- and second-phases of insulin secretion. While attenuated abundances of VAMP2, SNAP25 and Syntaxin 1 are reported in islets from human T2D individuals and from diabetic rodent models, the abundance of Syntaxin 4 is largely untested.

Syn4 is a SNARE required for biphasic insulin secretion. Syntaxin 4 homozygous (−/−) null mice die early in embryogenesis, apparently due to a requirement for Syn4 in the fusion of the GLUT8-containing vesicle with the plasma membrane in the mouse blastocyst. However, Syntaxin 4 heterozygous (−/+) knockout mice are viable and exhibit insulin resistance and impaired insulin secretion. This insulin resistance is largely due to significantly reduced skeletal muscle glucose uptake and GLUT4 translocation, while insulin secretion deficit is attributed to decreased first- and second-phase insulin release. In addition to the expected 50% decrease in Syn4 protein in the Syn4 (−/+) mouse tissues, Munc18c protein levels were decreased in parallel, while no other protein levels were altered.

In particular embodiments described herein, the nucleic acid encoding Syntaxin 4 has a mutation that makes it constitutively active, called Syntaxin LE, having the mutations L173A/E174A. Syntaxin L173A/E174A provides a Syntaxin 4 in a constitutively active (unfolded) state, as described in JBC, 282: 16553-16566, (2007). Constitutively active Syntaxin 4 provides for more robust insulin secretion.

Syntaxin proteins are regulated by specific high-affinity binding partners (mammalian homologue of unc-18 [Munc18] proteins). Three Munc18 isoforms are involved in one or both phases of glucose-stimulated insulin release, Munc18-1, Munc18b and Munc18c. Only one isoform, Munc18c, is involved in insulin-stimulated GLUT4 vesicle translocation. Munc18c is the only isoform capable of pairing with Syn4, and this pairing is altered when Munc18c becomes tyrosine phosphorylated in response to physiologically relevant stimuli.

TABLE 1
Expression of v- and t-SNARE isoforms in adipose, skeletal muscle, and pancreatic β-cells.

| | Tissue (Localization | Function |
|---|---|---|
| v-SNARE | Pancreatic β-cells, adipocytes | Exocytosis of insulin granules (β- |
| VAMP2/Synaptobrevin | muscle (vesicles) | cells), GLUT4 vesicles at the PM |
| VAMP3/Cellubrevin | Pancreatic β-cells, adipocyte, | Exocytosis of insulin granules (β- |
| | muscle (vesicles) | cells), GLUT4 vesicles at the PM |
| VAMP4 | Adipocyte, muscle (TGN) | ND |
| VAMP5/myobrevin | Adipocyte, muscle (PM) | Myogenesis |
| VAMP7/TI-VAMP | Adipocyte (PM, endosome) | Osmotic shock-induced GLUT4 |
| | | translocation |
| VAMP8/Endobrevin | Pancreatic β cells, adipocyte | GLUT4 endocytosis, insulin |
| t-SNARE | (endosome) | secretion (β-cells) |
| Syntaxin 1A | Pancreatic β cells (PM) | First phase insulin secretion in |
| | | pancreatic β cells |
| Syntaxin 2/Epimorphin | Pancreatic β cells, adipocyte | ND |
| Syntaxin 3 | Pancreatic β cells, adipocyte (PM) | ND |
| Syntaxin 4 | Pancreatic β cells, adipocyte, | Both phases of insulin secretion |
| | muscle (PM) | (β-cells), GLUT4 translocation |
| Syntaxin 5 | Adipocyte (TGN) | GLUT4 endocytosis in adipocyte |
| Syuntaxin 6 | Adipocyte, muscle (TGN) | Putative involvement in GLUT4 |
| | | endocytosis (adipocytes) |
| Syntaxin 7 | Pancreatic β-cells, adipocyte | ND |
| | (endosomes) | |
| Syntaxin 8 | Adipocyte (endosomes) | ND |
| Syntaxin 10 | Muscle | ND |
| Syntaxin 12 | Adipocyte | ND |
| Syntaxin 16 | Adipocyte (TGN) | GLUT4 intracellular trafficking |
| SNAP23 | Pancreatic β-cells, adipocyte, | Exocytosis of insulin granules (β- |
| | muscle (PM) | cells), GLUT4 vesicles at PM |
| SNAP25 | Pancreatic β-cells | Exocytosis of insulin granules (β- |
| | | cells) |

SNARE, soluble N-ethylmaleimide-sensitive factor attachment protein receptor protein;
ND, no determined;
PM, plasma membrane;
TGN, trans-Golgi network;
ER, endoplasmic reticulum.

Decreased Munc-18c-Syn4 binding occurs concomitantly with increased binding of double C2 domain protein β (Doc2b) to Munc18c, with Doc2v preferentially binding tyrosine-phosphorylated Munc18c. Doc2b is a ubiquitously expressed soluble protein that can localize to the plasma membranes (PM) of β cells, adipocytes and skeletal muscles. Doc2b has also been shown to bind to Syn4 in clonal β cells and 3T3L1 adipocytes, and has been shown to alter membrane bending via binding to Syn4-based SNARE complexes in vitro. Despite the discrepant mechanistic data, there is support for the role of Doc2b as a limiting factor in GSIS and glucose uptake.

As demonstrated in the Examples herein, Syn4 protein is markedly reduced in human islets from type 2 diabetes (T2D) individuals (˜70%), and that upregulating expression of Syn4 improves the insulin secretory function of T2D islets retaining low levels of residual function by ˜2-fold. Syn4 is also shown to be limiting for peak insulin release from healthy human islets, as its overexpression boosted the efficiency of glucose-regulated biphasic insulin release by 100%. In a minimal islet transplant model in vivo, Syn4-enriched mouse islets more effectively attenuating streptozotocin (STZ)-induced diabetes than control islets, and did so without causing hypoglycemia.

Also described herein is the ability of Syn4 to maintain glucose homeostasis during aging. Both aging and T2D are accompanied by deleterious physiological changes, such as development of insulin resistance, diminished glucose tolerance, fat accumulation and loss of muscle mass. Despite a strong correlation between aging and T2D, the molecular events linking aging and T2D are unknown. As describe herein, Syn4 transgenic mice overexpressing Syn4 live ˜33% longer than controls, and show increased peripheral insulin sensitivity, even at ages where controls show age-related insulin resistance. With Syn4 transgenic mice spending more hours of each day under normoglycemic conditions, Syn4 slows multiple aspects of aging related to glucose homeostasis.

Further, it is shown herein that Doc2b transgenic mice cleared glucose substantially faster than wildtype mice, correlating with enhancements in both phases of insulin secretion and peripheral insulin sensitivity. The heightened peripheral insulin sensitivity observed in the Doc2b transgenic mice correlates with elevated insulin-stimulated GLUT4 vesicle accumulation in cell surface membranes of their skeletal muscle. Doc2b enrichment enhances syntaxin-4-SNARE complex formation in skeletal muscle cells, and leads to both increased insulin secretion efficiency and increased insulin sensitivity.

These results show that overexpressing Syn4, Doc2b, or both, improves whole body glucose homeostasis by increasing insulin secretion, increasing peripheral insulin sensitivity, or a combination thereof. Described herein are methods for improving whole body glucose homeostasis in a subject, comprising inducing overexpression of one or both of these genes in the subject. Overexpression of the Syn4 and Doc2b genes can be achieved in the subject by methods including cell therapy and gene therapy.

A method for improving whole body glucose homeostasis may further comprise inducing overexpression of the Munc18c gene in the subject. In a preferred embodiment, Munc18c is overexpressed concomitantly with Syn4. Increased expression of Munc18c in islet cells is shown to increase insulin secretion efficiency, albeit to a lesser extent than Syntaxin 4. Therefore, upregulation of Syn4 and Munc18c in islet cells will have a synergistic effect in increasing efficiency of insulin release.

The methods and materials described herein can be used to improve whole body glucose homeostasis in any subject in need thereof. The subject can be of a variety of genus and species, including but not limited to the groups of human, canine, rodent, primate, swine, equine, sheep, and feline. Animals from each of these groups have been shown to suffer from insulin-related diseases and conditions, including type 1 diabetes, type 2 diabetes, chronic pancreatitis, pancreatectomy, insulin resistance, prediabetes, and age-related insulin resistance. Therefore, the methods and materials described herein can be used to prevent development of an insulin-related disease or condition in a subject, slow or halt progression of an insulin-related disease or condition in a subject, or treat an insulin-related disease or condition in a subject. For example the methods and materials described herein can be used to manage diabetes (type 1 or type 2) in dogs or cats, and to help prevent or treat insulin resistance, including age-related insulin resistance, in horses. The materials and methods described herein can also be used to promote longevity in mice, thereby allowing long-range laboratory studies. In a preferred embodiment, the materials and methods described herein are utilized to treat a human suffering from an insulin-related disease or condition (e.g., diabetes).

The methods and materials described herein improve whole body glucose homeostasis at least in part by improving insulin sensitivity, insulin secretion, or both in a subject. Wherein Syn4 and/or Doc2B are overexpressed in peripheral tissue, such as skeletal muscle, insulin sensitivity is increased. Wherein these genes are overexpressed in β cells, insulin secretion is increased, or improved. Improvements are considered any increase in glucose sensitivity, glucose secretion, or both, as determined by comparing sensitivity and/or secretion in a subject before and after a method described herein has been performed on the subject. Both insulin sensitivity and secretion may be determined by any method known in the art, including but not limited to hyperinsulinemic euglycemic clamp, modified insulin suppression test, homeostatic model assessment, quantitative insulin sensitivity check index, C-peptide test, deconvolution method, and combined method.

Overexpression of a gene (Syn4, Doc2B, Munc18c) can be achieved by either cell therapy, gene therapy, or a combination thereof.

In one aspect, a cell therapy for improving whole body glucose homeostasis in a subject is provided, comprising administering to the subject cells overexpressing Syn4, Doc2b, or both. Where the cells overexpress Syn4, Munc18c can also be overexpressed. Overexpression of a gene chosen from Syn4, Doc2B, and Munc18c can be achieved by introducing into the cells a nucleic acid molecule encoding the chosen gene. Overexpression of a gene in the cells can be achieved by introducing into the cells a nucleic acid having a sequence identity at least 80% identical to SEQ ID NO: 1 (human Syn4), SEQ ID NO: 2 (mouse Syn4), SEQ ID NO: 3 (human Syn4^{LE}), SEQ ID NO: 4 (mouse Syn4^{LE}), SEQ ID NO: 5 (human Doc2b), SEQ ID NO: 6 (mouse Doc2b), SEQ ID NO: 7 (human Munc18c), and SEQ ID NO: 8 (mouse Munc198c).

In particular embodiments, the nucleic acid can have a sequence identity at least 80%, at least 85%, at least 90%, at least 95%, at least 96%, at least 97%, at least 98%, at least 99%, or 100% identical to SEQ ID NO: 1 (human Syn4), SEQ ID NO: 2 (mouse Syn4), SEQ ID NO: 3 (human Syn4^{LE}), SEQ ID NO: 4 (mouse Syn4^{LE}), SEQ ID NO: 5 (human Doc2b), SEQ ID NO: 6 (mouse Doc2b), SEQ ID NO: 7 (human Munc18c), or SEQ ID NO: 8 (mouse Munc198c).

More than one gene can be upregulated or expressed in the cells at once. For example, Syn4 and Doc2b can be overexpressed at the same time, Syn4 and Munc18c can be overexpressed at the same time, Doc2b and Munc18c can be expressed at the same time, or Syn4, Munc18c, and Doc2b can all be overexpressed in the cells at the same time.

One or more nucleic acids can be introduced into the cells by any method known in the art, including but not limited to: transduction utilizing a viral vector; transfection, including chemical (e.g. liposome)-mediated transfection, electroporation, sonoporation, impalefection, optical transfection, and particle-based transfection (gene gun); and transformation, including chemical-mediated transformation and electroporation. One of skill in the art will recognize that the nucleic acids disclosed herein can be transferred to a cell to be administered to a subject, thereby resulting in upregulation or overexpression of a gene, and thus a gene product, in the cell.

In particular embodiments, the introduction of nucleic acid(s) into the cells to be administered to a subject is by transduction via a viral vector. The viral vector can be any viral vector suitable for transduction, including but not limited to a retroviral vector, adenovirus, herpes simplex virus, lentivirus, poxvirus, adeno-associated virus, and recombinant adeno-associated virus (rAAV). In a preferred embodiment, the viral vector is recombinant adeno-associated virus.

The nucleic acid, whether naked DNA or incorporated into a vector, may optionally be operably linked to a promoter. The promoter can be chosen to affect transcription and/or translation of the nucleic acid, and is ideally functional in a desired host cell. The promoter can be tissue specific, promoting transcription and/or translation only in particular tissues or cells. In certain aspects, it may desirable to prevent upregulation or overexpression of a gene outside of the cells in which upregulation or overexpression was initially induced.

The cells in which overexpression of one or more genes is to be induced can be autogenic, allogenic, or xenogenic, originating from the same subject on which the method is being performed, a different subject of the same species as that on which the method is being performed, and a subject of another species, respectively. The cells can be any type of cell capable of either secreting or responding to insulin, including but not limited to β (islet) cells, synthetic or semi-synthetic cells, transgenic cells, insulin producing cells derived from stem cells (differentiated stem cells) or precursor cells, and insulin producing cells derived from reprogrammed cells. Methods for differentiating stem cells into β cells, differentiating precursor cells into β cells, and reprogramming other cells, such as pancreatic α cells, into β cells have been developed; see, for example, US20110280842, describing methods of reprogramming cells into β cells, and WO2000078929, describing methods of dedifferentiating pancreatic cells and obtaining pancreatic islet cells from the dedifferentiated pancreatic cells.

Cells can be obtained from a subject themselves, a cadaver donor, a donor from another species, or any other source of donor β cell. Alternatively, β cells can be derived from stem cell differentiation, precursor cells, reprogramming of other cell types, or any other method known to produce β cells. β cells can be maintained in culture for a period of time under conditions allowing for cell growth and division, thereby increasing the number of β cells; see, for example, US20130023491, describing methods of increasing β-cell replication in a population of cells.

β cells can be isolated from a subject or donor, induced to overexpress Syn4, Doc2b, Munc18c, or a combination thereof, and administered to the subject. Administration can be undertaken by any means suitable to introduce cells to a subject, including intravenous administration and transplantation. As shown in the Examples below, intravenous administration of cells overexpressing Syn4 should not pose any systemic problems, as while beneficial when upregulated or overexpressed in pancreas and skeletal muscle, Syn4 is relatively inert in other tissues such as adipose tissue. Alternatively, cells overexpressing a nucleic acid described herein can be transplanted at a targeted site where improved insulin secretion and/or sensitivity is desired. The cells can be transplanted in, for example, pancreatic tissue, liver tissue, skeletal muscle tissue, adipose tissue, brain tissue, heart tissue, spleen tissue, kidney tissue, and lung tissue.

When isolating β cells from the subject or a donor, separation or isolation of the β cells from the connective matrix and remaining exocrine tissue is advantageous. Transplanting β cells rather than complete pancreatic tissue has the distinct advantages of ease of transplantation, and the elimination of the pancreatic exocrine function of the donor tissue involving secretion of digestive enzymes. Liberating islets from pancreatic exocrine tissue is an initial and crucial step that influences successful β cell transplantation.

A widely used method for transplanting β cells is known as the “Edmonton Protocol.” The Edmonton Protocol transplants healthy β cells into diabetic patients. β cell transplantation using the Edmonton Protocol is described in Shapiro et al. Transplantation Proceedings, 33, pp. 3502-3503 (2001); Ryan et al., Diabetes, Vol. 50, April 2001, pp. 710-719; and Ryan et al., Diabetes, Vol. 51, July 2002, pp. 2148-2157. Once in the liver, the cells develop a blood supply and begin producing insulin. The Edmonton Protocol, or any other islet cell transplantation protocol, can be used to administer cells overexpressing Syn4, Doc2b, Munc18c, or a combination thereof.

In addition to administration of the cells overexpressing Syn4, Doc2b, Munc18c, or a combination thereof, a cell therapy described herein can further comprise administration of anti-inflammatory and/or immunosuppressive drugs prior to, concurrently, or following the administration of the cells. Suitable drugs include but are not limited to cyclosporin, FK506, rapamycin, corticosteroids, cyclophosphamide, mycophenolate mofetil, leflunomide, deoxyspergualin, azathioprine, OKT-3, and the like. The inflammatory response associated with islets is a primary cause of early damage to β cells and graft loss after transplantation. Harmful inflammatory events can occur during pancreas procurement (from the subject or a donor), pancreas preservation, islet isolation, and islet infusion. Controlling pre- and peritransplant islet inflammation improves posttransplant islet survival, enhancing the benefits of the transplantation. Immunosuppressive medications can be helpful in preventing rejection of the transplant.

Use of anti-inflammatory and/or immunosuppressive drugs can also be beneficial during systemic, intravenous administration of the cells overexpressing Syn4, Doc2b, Munc18c, or a combination thereof.

In a particular embodiment of the cell therapy method described herein, β cells overexpressing Syn4 are generated and transplanted in a tissue of the subject. The tissue can be any tissue described above, including kidney tissue.

In other particular embodiments of the cell therapy method, the cells are autologous β cells isolated from the subject, transduced to induce overexpression of Syntaxin 4, and transplanted back into the subject.

A gene therapy for improving whole body glucose homeostasis in a subject is also provided herein, comprising administering to the subject at least one nucleic acid associated with the gene Syn4 or the gene Doc2b. In certain instances, it may be desirable to administer two or more nucleic acids associated with Syn4 and Doc2b. Wherein the nucleic acid comprises Syn4, a nucleic acid associated with Munc18c can be further administered to the subject.

Overexpression of a gene chosen from yn4, Doc2B, and Munc18c can be achieved in a subject by administering to the subject a nucleic acid having a sequence identity at least 80% identical to SEQ ID NO: 1 (human Syn4), SEQ ID NO: 2 (mouse Syn4), SEQ ID NO: 3 (human Syn4^{LE}), SEQ ID NO: 4 (mouse Syn4^{LE}), SEQ ID NO: 5 (human Doc2b), SEQ ID NO: 6 (mouse Doc2b), SEQ ID NO: 7 (human Munc18c), and SEQ ID NO: 8 (mouse Munc198c).

In particular embodiments, the nucleic acid can have a sequence identity at least 80%, at least 85%, at least 90%, at least 95%, at least 96%, at least 97%, at least 98%, at least 99%, or 100% identical to SEQ ID NO: 1 (human Syn4), SEQ ID NO: 2 (mouse Syn4), SEQ ID NO: 3 (human Syn4^{LE}), SEQ ID NO: 4 (mouse Syn4^{LE}), SEQ ID NO: 5 (human Doc2b), SEQ ID NO: 6 (mouse Doc2b), SEQ ID NO: 7 (human Munc18c), or SEQ ID NO: 8 (mouse Munc198c).

More than one nucleic acid can be administered to the subject at once. For example, nucleic acids associated with Syn4 and Doc2b can be administered at the same time, nucleic acids associated with Syn4 and Munc18c can be administered at the same time, nucleic acids associated with Doc2b and Munc18c can be administered at the same time, or nucleic acids associated with Syn4, Munc18c, and Doc2b can all be administered to the subject at the same time

The nucleic acids described herein, once administered to the subject, result in the upregulation or overexpression of Syn4, Doc2b, Munc18c, or combinations thereof, thereby improving whole body glucose homeostasis.

The nucleic acids can be administered as naked DNA, or as part of a plasmid or vector. Where nucleic acids are present in a vector, the vector may be viral or non-viral. DNA- and RNA-liposome complex formations are examples of useful non-viral vectors. Such complexes comprise a mixture of lipids which bind to genetic material (DNA or RNA), providing a hydrophobic coat which allows the genetic material to be delivered into cells. Liposomes which can be used include DOPE (dioleyl phosphatidyl ethanol amine) and CUDMEDA (N-(5-cholestrum-3-β-ol 3-urethanyl)-N′,N′-dimethylethylene diamine).

When Syntaxin 4, Munc18c and/or Doc2b is administered using a liposome, it is preferable to first determine in vitro the optimal DNA:lipid ratio and the absolute concentration of DNA and lipid as a function of cell death and transformation efficiency for the particular type of cell to be transformed. These values can then be used, or extrapolated for use, in vivo administration. The in vitro determination of these values can be readily carried out using techniques known in the art. Some other examples of non-viral vectors include non-lipid cationic polymers [polyethylenimine (PEI), polyamidoamine (PAMAM), poly-L-lysine], hemagglutinating virus of Japan-envelope (HVJ-E, an inactivated Sendai virus envelope), cationic liposomal lipid (Lipofectamine), and cationic non-liposomal lipids (Effectene), were developed for favorable transfection efficiency in gene transfer.

Other non-viral vectors can also be used in accordance with the present disclosure. These include chemical formulations of nucleic acids coupled to a carrier molecule or other molecule which facilitates delivery to target cells and tissues for the purpose of altering the biological properties of the host cells (e.g., increasing insulin secretion or sensitivity). Exemplary protein carrier molecules include antibodies specific to the islet cells or receptor ligands, i.e., molecules and peptides capable of interacting with receptors associated with a cell of a targeted secretory gland.

Other methods for delivering nucleic acids to target cells and tissues include, for example, lipoplex condensation and encapsulation, polymersome condensation and encapsulation, polyplex complex formation, dendrimer complex formation, inorganic nanoparticle complex formation, and cell penetrating peptide complex formation. These are further examples of possible chemical modifications of a nucleic acid described herein, facilitating delivery of the nucleic acid to a target cell or tissue. This delivery may be further facilitated by incorporating a tissue specific peptide, thereby targeting the chemically modified nucleic acid to a particular tissue (e.g., pancreas or skeletal muscle).

Nucleic acids describe herein can also be administered to a subject in a viral vector. The viral vector can be a lentiviral vector, such as a human immunodeficiency virus (HIV) vector or a simian immunodeficiency virus (SIV) vector, bovine immunodeficiency virus, equine infectious anemia virus, feline immunodeficiency virus, puma lentivirus, caprine arthritis encephalitis virus, or visna/maedi virus. “HIV” is meant to include all clades and/or strains of human immunodeficiency virus 1 (HIV-I) and human immunodeficiency virus 2 (HIV-2). Likewise, the viral vectors can be self-inactivating (SIN) vectors, which have an inactivating deletion in the U3 region of the 3′ LTR. Such a deletion may include the deletion of the enhancer and/or promoter. SIN vectors are engineered so that transcription of the target gene can only be driven by an internal promoter once the expression cassette is integrated into the genome. Given the deletion in the U3 region, expression of Syn4, Munc18c, and/or Doc2b can be driven by a promoter. In certain embodiments, the gene transfer vector is selected from a retroviral vector, adenovirus, lentivirus, adeno-associated virus, recombinant adeno-associated virus, poxvirus, herpes simplex virus, and the like.

Self-inactivating retroviral vectors can also be used. The self-inactivation of the retroviral vector minimizes the risk that Replication Competent Retrovirus (RCRs) will emerge. It also reduces the likelihood that cellular coding sequences located adjacent to the vector integration site will be aberrantly expressed, either due to the promoter activity of the 3′ LTR or through an enhancer effect. Finally, a potential transcriptional interference between the LTR and the internal promoter (used for expression in tissue/cells of interest) driving the transgene is prevented by the SIN vector design.

In a preferred embodiment, the vector is an adeno-associated virus, due to the vector's minimal immunogenicity and high safety profile. Recombinant adeno-associated virus (rAAV) vectors appear to offer a vehicle for safe, long-term therapeutic gene transfer afforded through the propensity of rAAV to establish long-term latency without deleterious effects on the host cell and the relative non-immunogenicity of the virus or viral expressed transgenes.

The nucleic acids described herein, whether naked DNA, or incorporated into a plasmid or vector, can be administered to the subject by any means known in the art, including but not limited to intravenous injection, direct organ or tissue injection, organs surface instillation, intra-arterial injection, intraportal injection, and retrograde intravenous injection. These are all methods known in the art for gene delivery in a subject.

As described herein, Syn4 has either no effects or only positive effects when overexpressed in other tissue types, the nucleic acids can be administered systemically (e.g., intravenously). Doc2b is shown herein to have beneficial effects in increasing insulin sensitivity when expressed in skeletal muscle, so systemic delivery of Doc2b is also beneficial.

In order to target particular tissues, or to improve delivery of the nucleic acid, physical methods, including but not limited to electroporation, sonoporation, mechanical massage, and ultrasound can be used in conjunction with any particular method of administration. These physical methods have been shown in the art to enhance gene delivery by improving entry of the nucleic acid (whether or not in a vector) into cells of a target tissue.

Tissues beneficial for targeting by a method described herein include pancreatic tissue, skeletal muscle tissue, adipose tissue, brain tissue, heart tissue, liver tissue, spleen tissue, kidney tissue, and lung tissue.

Particular tissues can be further targeted by operatively linking a nucleic acid described herein to a tissue specific promoter, which can limit expression of Syn4, Doc2b, and/or Munc18 to particular tissues. Preferably the promoter is a strong, eukaryotic promoter. Exemplary eukaryotic promoters include promoters from cytomegalovirus (CMV), mouse mammary tumor virus (MMTV), Rous sarcoma virus (RSV), and adenovirus. More specifically, exemplary promoters include the promoter from the immediate early gene of human CMV and the promoter from the long terminal repeat (LTR) of RSV. Of these two promoters, the CMV promoter provides for higher levels of expression than the RSV promoter. There are a number of promoters known in the art that are capable of driving expression of Syn4, Doc2b, and/or Munc18cs.

The promoter can be tissue specific, such as a pancreas-specific promoter, i.e. the promoter facilitates specific expression of the nucleic acid to which it is operably linked when the construct is in the presence of a pancreas cell specific transcriptional activator protein. Examples of pancreas specific promoters include the insulin promoter and pancreas α-amylase promoters. Other beneficial promoters include for, example, skeletal muscle-specific promoters, kidney-specific promoters, and liver-specific promoters. The promoters can be derived from the genome of any mammal, and are preferably derived from a murine or a human source, more preferably from a human source.

Described herein are methods for improving the healthspan of a subject. These methods generally comprise inducing overexpression of Syntaxin 4 in the subject. Healthspan of an individual can be improved by overexpressing Syn4 in peripheral tissues, such as skeletal muscle, in β cells, or both. As used herein, “healthspan” refers to the overall health of the subject, wherein the subject is generally healthy and free from serious disease. By overexpressing Syn4 in a subject, insulin sensitivity and/or secretion may be maintained at healthy levels despite the subject being of an advanced age, obese, or both. By increasing, or at least maintaining insulin sensitivity and/or secretion in these individuals, healthspan is improved. In particular embodiments, healthspan is improved in a human or mouse.

The methods and materials disclosed herein can also be used to prolong the lifespan of a subject. By maintaining or improving insulin secretion and/or sensitivity, lifespan of a subject may be extended. In certain embodiments, the lifespan of a mouse is extended by a method described herein.

Also described herein are pharmaceutical compositions comprising at least one nucleic acid described herein. Such compositions can be used in gene therapy methods described herein for improving whole body glucose homeostasis.

A pharmaceutical composition comprises at least one nucleic acid associated with a gene selected from the group consisting of: Syntaxin 4; and Doc2b, wherein the at least one nucleic acid is operably linked to a promoter. The pharmaceutical composition can further comprise a nucleic acid associated with Munc18c. As described above, the promoter is functional in eukaryotic cells, and can be tissue specific.

The nucleic acids associated with any of the genes Syn4, Doc2b, and Munc18c are the same as those described above. The nucleic acids have a sequence identity at least 80% identical to at least one of SEQ ID NO: 1 (human Syn4), SEQ ID NO: 2 (mouse Syn4), SEQ ID NO: 3 (human Syn4^{LE}), SEQ ID NO: 4 (mouse Syn4^{LE}), SEQ ID NO: 5 (human Doc2b), SEQ ID NO: 6 (mouse Doc2b), SEQ ID NO: 7 (human Munc18c), and SEQ ID NO: 8 (mouse Munc198c).

In certain aspects, the nucleic acids have a sequence identity at least 80%, at least 85%, at least 90%, at least 95%, at least 96%, at least 97%, at least 98%, at least 99%, or 100% identical to at least one of SEQ ID NO: 1 (human Syn4), SEQ ID NO: 2 (mouse Syn4), SEQ ID NO: 3 (human Syn4^{LE}), SEQ ID NO: 4 (mouse Syn4^{LE}), SEQ ID NO: 5 (human Doc2b), SEQ ID NO: 6 (mouse Doc2b), SEQ ID NO: 7 (human Munc18c), or SEQ ID NO: 8 (mouse Munc198c).

The at least one nucleic acid of the pharmaceutical composition can be present as naked DNA or in a vector. Suitable vectors are discussed above.

The pharmaceutical composition can comprise a pharmaceutically acceptable excipient. The excipient can be a natural or synthetic substance, and can act as a filler or diluents for the at least one nucleic acid, facilitating administration to the subject. The excipient can also facilitate nucleic acid uptake into a target cell, or otherwise enhance overexpression of Syn4, Doc2b, and/or Mun18c in the subject.

### Examples

The methods and embodiments described herein are further defined in the following Examples. Certain embodiments of the present invention are defined in the Examples herein. It should be understood that these Examples, while indicating preferred embodiments of the invention, are given by way of illustration only. From the discussion herein and these Examples, one skilled in the art can ascertain the essential characteristics of this invention and without departing from the spirit and scope thereof, can make various changes and modifications of the invention to adapt it to various usages and conditions.

### Example I

### Syntaxin 4 Upregulation—Materials and Methods

Mice.

The rat Syntaxin 4 cDNA inserted into the pCombi-CMV targeting vector was used to generate heterozygous transgenic mice on the C57 BL/6J strain background. Syntaxin 4 over-expressing transgenic mice were generated and maintained as heterozygous for the transgene on the C57BL/6J strain background and paired littermates used as controls, according to the Indiana University School of Medicine Laboratory Animal Research Center guidelines for use and care of animals. NSG (NOD/SCID-IL2R-γ-null) and NOD-SCID mice were obtained from the Indiana University In vivo Therapeutics Core.

Human Islet Perifusion.

Human islets were obtained through the Integrated Islet Distribution Program, IIDP. Human islets isolated <36 hr from isolation were accepted (Table 2), allowed to recover in CMRL medium for 2 h, then handpicked. Islets were immediately transduced (MOI=100) with Control-Ad or Syntaxin 4-Ad CsCl-purified particles for perifusion analysis and insulin secretion quantitated by radioimmunoassay (Millipore; Billerica, Mass.). Insulin content from donor islets solubilized in NP40-lysis buffer was quantified relative to total islet protein content.

Islet Morphometry.

β cell mass in Syntaxin 4 transgenic (Tg) and littermate wild-type (WT) mice were determined. Pancreata from five-month old male WT or Syntaxin 4 Tg mice were fixed with 10% formalin (neutralized buffer), paraffin embedded, and longitudinally sectioned at 5-μm thickness and 100-μm intervals for insulin staining and counterstaining with hematoxylin. Digital images were acquired on an Axio-Observer Z1 microscope (Zeiss) fitted with an AxioCam high resolution color camera and associated software used to calculate β cell area. Data are representative of 16-18 sections per pancreas and 3 pancreata from each group.

Immunoblotting.

Human islets were lysed in 1% NP40 lysis buffer and proteins resolved by 10% SDS-PAGE followed by transfer to PVDF membranes for immunoblotting for visualization by ECL-based detection using a Chemi-Doc documentation imaging system (Bio-Rad; Hercules, Calif.).

Transplantation.

Islets were isolated as described in Stull et. al. J Vis Exp (2012), 67 from donor mice (10-14 weeks old), and transplanted under the renal capsule of recipient 8 week-old streptozotocin (STZ, 180 mg/kg) diabetic NSG mice in groups of 100 islets per recipient as described in Zmuda et al J Vis Exp (2011) 50. Random (non-fasted) blood glucose measurements were obtained on days 2, 6 and 14. On day 15 kidneys harboring transplanted islets were surgically removed (nephrectomy) for measurement on day 18.

Statistical Analyses—

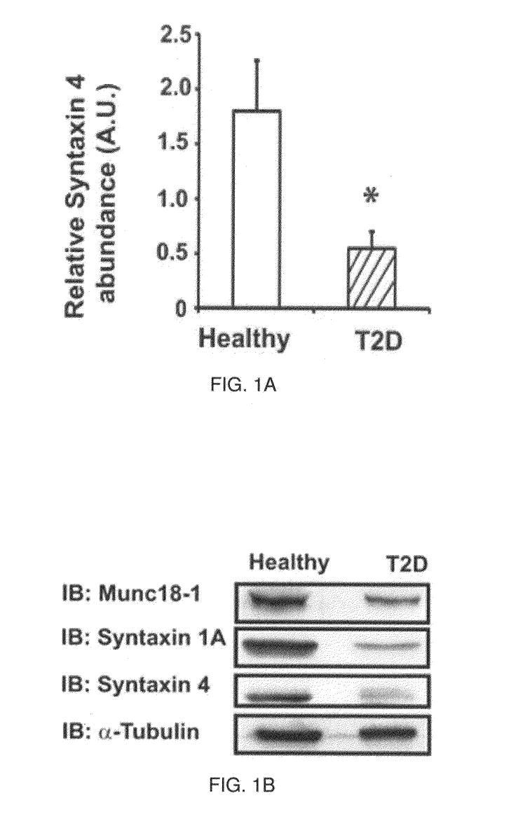

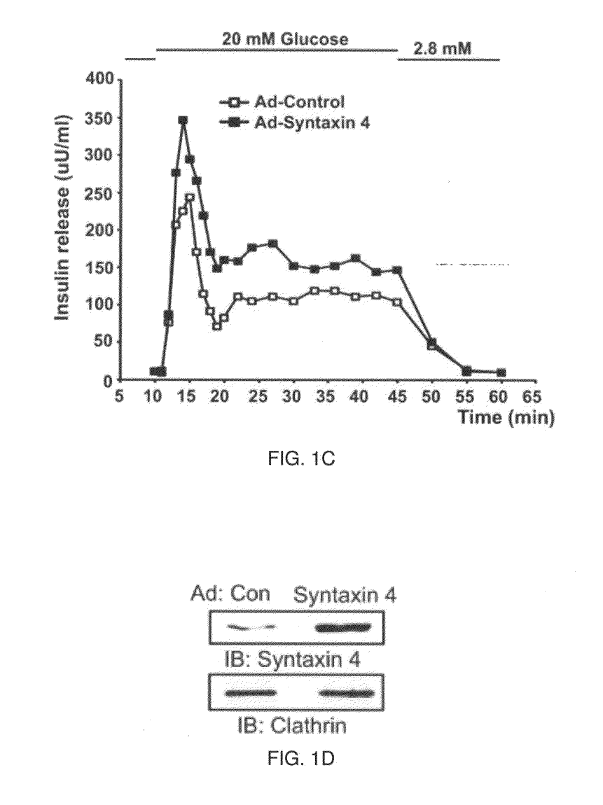

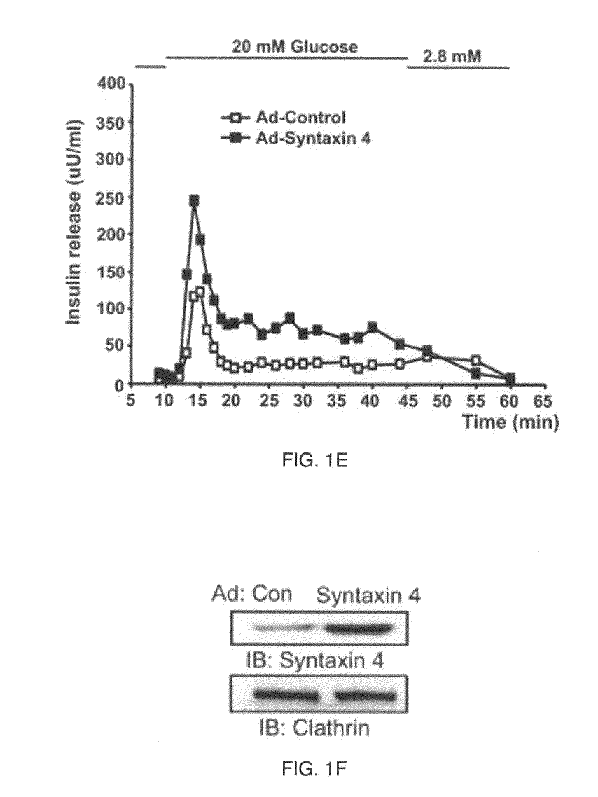

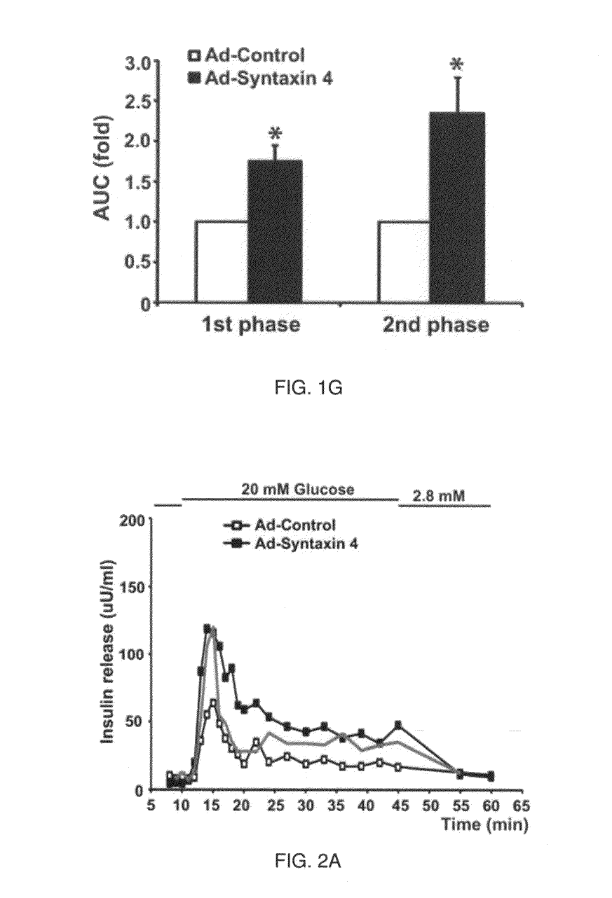

All values are presented as means±SE. Differences between two groups were analyzed by Student's unpaired t-test for independent samples; a 1 sample t-test was used for FIG. 1D.

### Example II

### Syntaxin 4 Protein Levels are Reduced in Diabetic Human Islets and are Limiting for Islet Function

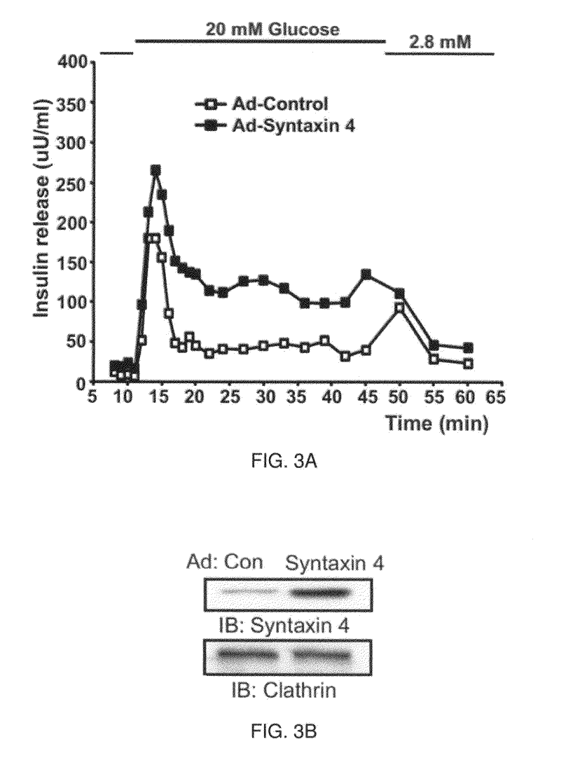

To assess Syntaxin 4 levels in human islets, islets from healthy and T2D individuals (cadaveric donors) were subjected to immunoblotting, revealing a reduction in Syntaxin 4 protein in islets from T2D subjects of about 70% (FIGS. 1A-1B). To determine if over-expression of Syntaxin 4 in healthy human islets recapitulates the beneficial effects upon biphasic insulin release observed with rodent islets, human islets were transduced with Ad-Syntaxin 4 or Ad-Control viral particles and over-expression validated by immunoblotting. Glucose-stimulated insulin secretion (GSIS) was substantially elevated in each of three independent batches of healthy donor islets over-expressing Syntaxin 4 (FIGS. 1C-1F, FIGS. 3A-3B); basal insulin release was normal. Peak amplitudes of each phase in this small study tended to be decreased with increased age of the human islet donor. Area under the curve (AUC) analysis showed Ad-Syntaxin 4-expressing human islets releasing about 80% and 130% more insulin during the first and second phases of GSIS, respectively, compared with Ad-control islets from the same donor (FIGS. 1E-1F). These data indicate that Syntaxin 4 is limiting for biphasic GSIS.

TABLE 2
Human Islet Donor Profiles

| | | | | | Islet | Islet | HbA1c/BG/yrs | Experimental |
|---|---|---|---|---|---|---|---|---|
| FIG. NO: | Gender | Age | BMI | Race | purity | viability | with diabetes | Use of islets |
| 1A | F | 50 y | 32.9 | Caucasian | 85 | NR | NA | IB, insulin |
| | | | | | | | | content |
| 1A | M | 39 y | 33.0 | Caucasian | 80 | 90 | NA | IB |
| 1A | M | 48 y | 33.5 | Caucasian | 95 | 94 | NA | IB |
| 1A | F | 54 y | 30.6 | Caucasian | 95 | 98 | NA | IB |
| 1A | M | 44 y | 35.4 | Caucasian | 85 | NR | NA | IB |
| text | M | 40 y | 28.3 | Caucasian | 75 | 90 | NA | Insulin content |
| 1A* | M | 54 y | 35.8 | Caucasian | 95 | 88 | 5.8/188/NR | IB |
| 1A*, | F | 54 y | 48.5 | African | 90 | 90 | NR/170/5 | Perifusion, |
| 2A* | | | | American | | | | Insulin |
| | | | | | | | | content, IB |
| 1A*, | F | 45 y | 28.5 | Hispanic | 85 | 83 | 14.5/748/10 | Perifusion, |
| 2B* | | | | | | | | insulin |
| | | | | | | | | content, IB |
| 1A*, | M | 43 y | 37.0 | NA | 80 | 90 | 6.4/351/10 | Perifusion, IB |
| 4A | | | | | | | | |
| 1A*, | M | 42 y | 34.9 | Hispanic | 85 | 96 | 9.1/271/5 | Perifusion, |
| 4B*, | | | | | | | | insulin |
| | | | | | | | | content, IB |
| 1B, D | M | 25 y | 29.3 | Hispanic | 99 | 99 | NA | Perifusion |
| 1C, D | F | 59 y | 28.2 | Caucasian | 92 | 92 | NA | Perifusion |
| 1D, T1 | F | 51 y | 21.2 | Caucasian | 85 | 92 | NA | Perifusion, |
| | | | | | | | | insulin content |
| 4C | M | 58 y | 48.9 | Caucasian | 83 | 96 | NR/NR/5 | Perifusion |
| 4D* | NR | 43 y | 27.0 | Hispanic | 80 | 90 | 11.1/82‡/2 | IB, Perifusion, |
| | | | | | | | | insulin content |
| 4A | M | 25 | 35.7 | Pacific | 85 | 90 | NA | Transplantation, |
| | | | | Islander | | | | insulin |
| | | | | | | | | content |
| 4B | M | 41 | 29.3 | Hispanic | 90 | 93 | NA | Transplantation |

*= type 2 diabetic donors. History of anti-diabetic therapy not available.
‡= at time of procurement.
NR; information not reported by islet centers.
NA; not applicable (not reported for non-diabetic donors).
BG; blood glucose (mg/dL) provided by islet center.
IB = immunoblot.

### Example III

### Syntaxin 4 Upregulation can Improve the Function of Diabetic Islets

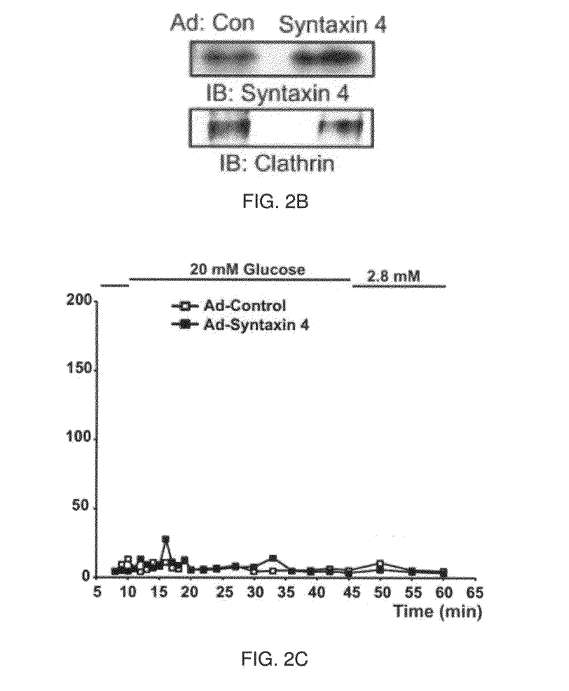

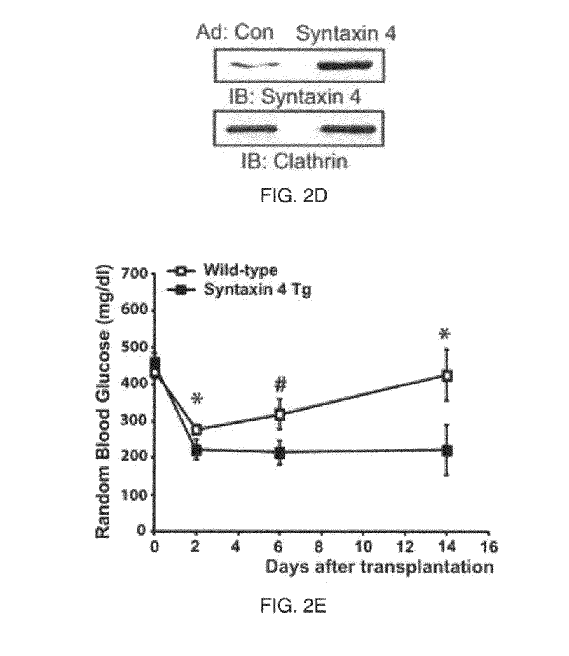

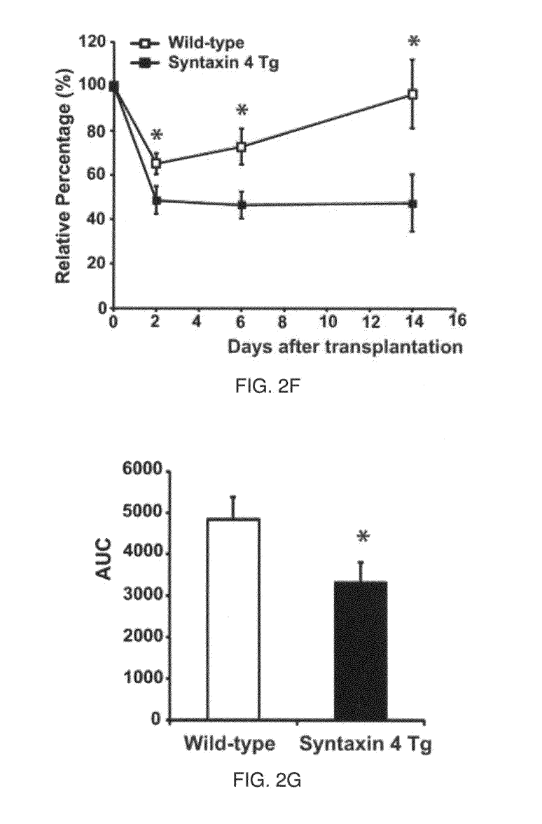

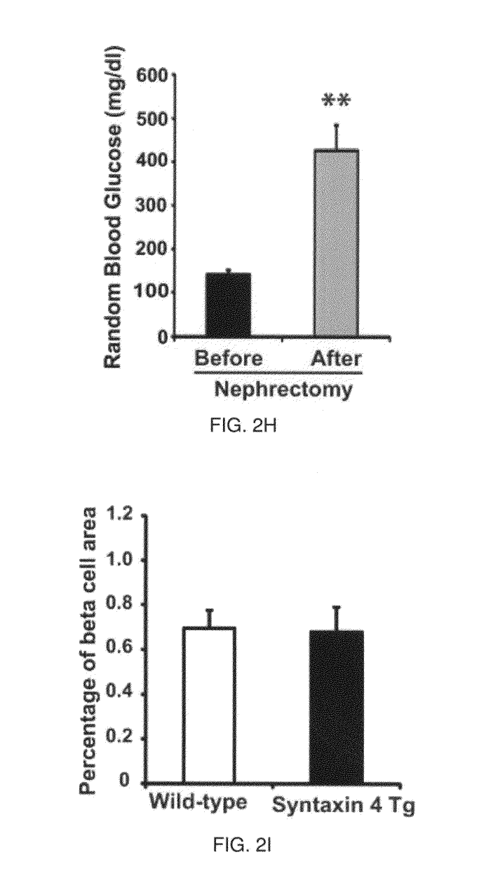

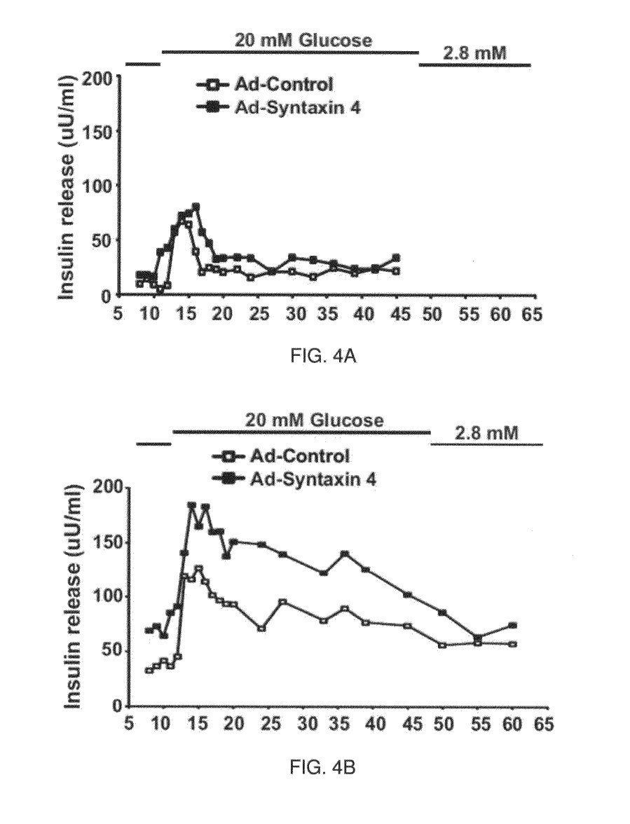

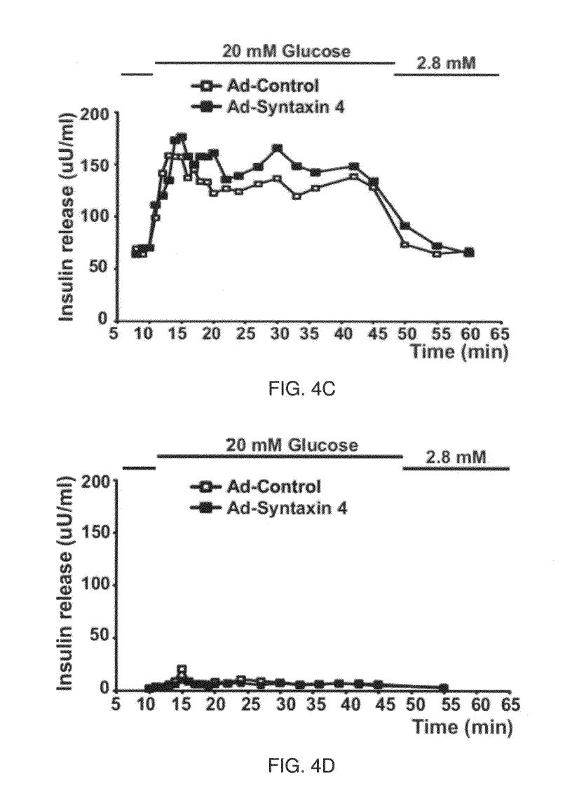

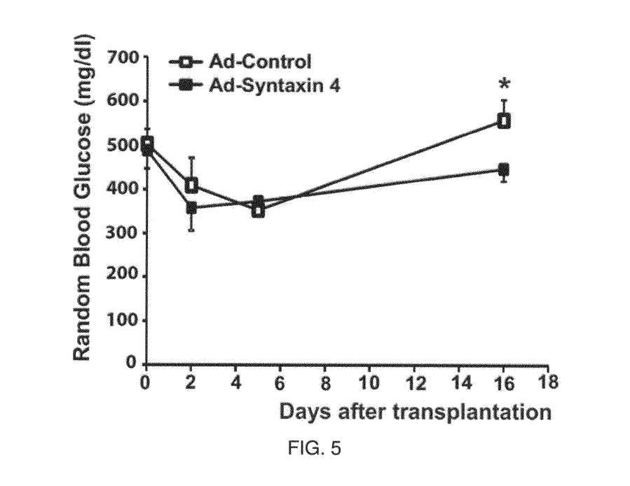

Given the boost afforded to normal healthy human islets, it was determined whether replenishment of Syntaxin 4 in human islets of T2D individuals would restore islet function. Six independent batches of human T2D islets were obtained and transduced with Ad-Syntaxin 4 or Ad-Control for parallel perifusion analysis as in FIGS. 1C-1F. Four of the six human T2D islet batches displayed an attenuated response to stimulatory glucose (Ad-Control), with a first phase peak amplitude height at about half that of the lowest of the three healthy islets cases of FIG. 1 (FIG. 2A, FIGS. 4A-4C). In these four cases Syntaxin 4-enriched T2D islets showed enhanced first and second phases, with one case showing improvements matching peak amplitudes seen in an age-matched healthy individual (FIG. 2A). However, islets from the remaining two T2D individuals had virtually undetectable levels of insulin release (FIG. 2B, FIG. 4D), consistent with the phenotypes of these two T2D donors having the highest HbA1c values (14.5 and 11.1%) of all donors assessed (Table 2). Insulin content of one batch of the unresponsive T2D islets (FIG. 4D) was 183 μU/μg protein, compared with the responsive T2D donor (FIGS. 2A-2B) at 382 μU/μg protein; both were lower than insulin contents of healthy age-matched donor islets at 1,170 μU/μg. These data indicate the effectiveness of Syntaxin 4 enrichment apply to cases of well-controlled or early-onset diabetes harboring a base level of functional β cells/insulin content.

### Example IV

### Syntaxin 4 Enrichment of Islet Cells Reverse Diabetes In Vivo

To evaluate the ability of Syntaxin 4 enrichment to reverse diabetes in vivo, a minimal islet transplant model system was used. Only 100 islets from male Syntaxin 4 transgenic or littermate wild-type mice were implanted under the renal capsule of recipient 8 week-old diabetic (high-dose STZ, 180 mg/kg) NSG mice. Within 2 days both wild-type and Syntaxin 4-islet recipient mice showed significantly improved blood glucose levels, yet only the Syntaxin 4-islet recipients maintained the improvement throughout the 14 day period (FIG. 2E); wild-type-islet recipients reverted back to pre-transplant glucose levels of 450 mg/dL by day 14. When normalized to starting glucose levels, the Syntaxin 4-islet recipient mice showed an initial and sustained 50% reduction in blood glucose, whereas wildtype-islet recipient mice showed only a 35% initial reduction, which was abolished by day 14 (FIG. 2F).

AUC analysis of glycemia following transplantation shows a 35% improvement with Syntaxin 4-donor islets compared with wild-type-donor islets (FIG. 2H). Nephrectomies performed on day 15 confirmed that the improvements in glycemia were attributable to the Syntaxin 4-donor islets (insulin secreted from the transplanted donor mouse islets cannot be discerned from those of the recipient mouse), as Syntaxin 4-islet recipients reverted back to pre-transplant hyperglycemic levels (FIG. 2H). Syntaxin 4 transgenic and wild-type mice had equivalent relative β cell areas (FIG. 2I), indicating that Syntaxin 4 enrichment enhances secretion via increasing islet function. Since Syntaxin 4 transgenic mice show normal fasting insulinemia and appropriate responsiveness during a glucose challenge, and the islets ex vivo exhibit normal biphasic secretory patterning, islet function was not unregulated.

### Example V

### Doc2b Enrichment Studies—Materials and Methods

Materials.

The rabbit anti-Munc18c and rabbit anti-Syn4 antibodies were generated in-house. The rabbit anti-Syntaxin 4 (for immunoprecipitation), SNAP-23, SNAP25, and Doc2b antibodies were purchased from Chemicon (Temecula, Calif., U.S.A.), Affinity Bioreagents (Golden, Colo., U.S.A.) and Abcam (Cambridge, Mass., U.S.A.) respectively. The Munc18-1, VAMP2 and Syn1A antibodies were acquired from Synaptic Systems (Gottingen, Germany) and Sigma (St. Louis, Mo., U.S.A.), respectively. The Akt and phosphoserine (Ser 473)-specific Akt antibodies were purchased from Cell Signaling, Inc. (Beverley, Mass., U.S.A.). Goat anti-rabbit-HRP and anti-mouse-HRP secondary antibodies were purchased from Bio-Rad (Hercules, Calif., U.S.A.). Protein G+ agarose beads and anti-GLUT4 antibody were acquired from Santa Cruz Biotechnology (Santa Cruz, Calif., U.S.A.). The rat insulin radioimmunoassay kit was acquired from Millipore (Billerica, Mass., U.S.A.). Enhanced chemiluminescence (ECL) was purchased from Amersham Biosciences (Pittsburgh, Pa., U.S.A.). Humulin R was obtained from Eli Lilly (Indianapolis, Ind., U.S.A.).

Animals and In Vivo Procedures.

All studies involving mice followed the Guidelines for the Use and Care of Laboratory Animals The pUC-Combi^{CMV} vector described in Schultze et. al. Nat Biotechnol 1996, 14:499-503 (Dr. Ulli Certa, Hoffman-LaRoche, Switzerland) was used to generate tetracycline-repressible Doc2b over-expressing transgenic mice. The vector contains a tetO minimal promoter to drive the target gene (DOC2B in this case) in one direction, and a CMV promoter to drive the tet-transactivator gene in the other direction. This vector has been shown to drive expression primarily in pancreas, fat and skeletal muscle and not in brain, heart, liver, spleen, kidney or lung. Mice were generated and maintained on the C57BL/6J genetic background; breeders to expand the initial colony were purchased from Jackson Labs (Bar Harbor, Me., USA). Female Doc2b Tg and Wt mice (4-6 months old) were fasted for 6 h (08:00-14:00) for IPGTT and ITT analyses. Pancreatic mouse islets were isolated and perifusion performed. Skeletal muscle subcellular fractionation and immunoprecipitation assays were performed.

Generation of Doc2b Transgenic Mice.

The pUC-Combi^{CMV} plasmid used for generation of tetracycline-repressible Doc2b Tg mice contains the tetO minimal promoter to drive the Doc2b gene in one direction, and a CMV promoter to drive the transactivator gene in the other direction. The full-length cDNA for DOC2B carrying an N-terminal Myc tag was inserted into the vector at the PmeI site. The construct was linearized by digestion with NotI and microinjected into the nucleus of pre-implantation embryos. These embryos were then transferred into the oviduct of pseudo-pregnant C57BL/6J female mice by the Indiana University, School of Medicine Transgenic Animal Facility. Thirty eight pups were screened for the presence of the transgene using PCR of genomic DNA, with four founders resulting. Of the four lines, F5170, the line with the highest expression levels in pancreas, fat and skeletal muscle (˜2-3.5-fold) was selected for full phenotypic characterization. Genotypes determined by PCR on DNA from tail-biopsy specimens (primers for Tg: 5′ to 3′: #3-ggcagaggacaagtccctgg (SEQ ID NO: 9); #9-agaggattgagcgttggcac (SEQ ID NO: 10); and #10-acgacgaggcttgcaggatcataa (SEQ ID NO: 11). Primers for Wt: 5′ to 3′: O-ggaaagaaggcgaatggaag (SEQ ID NO: 12) and F-tcactccagggttttcatcc (SEQ ID NO: 13)), whereby the PCR product of the wild-type (Wt) allele was 500 bp and that of the Tg allele was 681 and 277 bp.

Cell Culture, Transient Transfection and Binding Assays.

Rat L6 muscle cells stably expressing GLUT4 with an exofacial myc-epitope (Dr. Amira Klip, Department of Biochemistry, University of Toronto, Canada) were cultured as previously described in Walker et al, J Biol Chem, 1990, 265:1516-1523. Myoblasts were electroporated (0.20 kV and 960 μFarad) with 150 μg of GFP vector or GFP-DOC2B plasmid DNA per 10 cm² dish. After electroporation, cells were allowed to adhere to plates for 48 hours. Cells were pre-incubated in serum-free media for 2 hour followed by stimulation for 5 minutes with 100 nmol/l insulin. Cells were harvested in 1% NP-40 lysis buffer and detergent lysates used for co-immunoprecipitation or GST-VAMP2 interaction assays.

Intraperitoneal Glucose Tolerance Test (IPGTT) and Insulin Tolerance Test (ITT).

Female C57BL/6J Doc2b Tg and Wt mice (4-6 months old) were fasted for 6 h (08:00-14:00) before the IPGTT. Following sample collection of fasted blood, animals were given glucose (2 g/kg body weight) by intraperitoneal injection, and blood glucose readings were taken at 30, 60, 90 and 120 min for IPGTT. For the ITT, female mice (4-6 months old) were fasted for 6 hrs, sampled for fasted blood and then injected intraperitoneally with Humulin R (0.75 U/kg body weight) and blood glucose readings taken at 15, 30, 60 and 90 min after injection. Blood was collected from the tail vein and measurement of blood glucose was performed using the Hemocue glucometer (Mission Viejo, Calif., U.S.A.).

Isolation, Culture and Perifusion of Mouse Islets.

Pancreatic mouse islets were isolated from pancreata of 10-14 week old female mice. After isolation, islets were cultured overnight in CMRL-1066 medium, and hand-picked into groups of 40 onto cytodex bead columns. The islets were pre-incubated in Krebs-Ringer bicarbonate buffer (10 mmol/l HEPES pH 7.4, 134 mmol/l NaCl, 5 mmol/l NaHCO₃, 4.8 mmol/l KCl, 1 mmol/l CaCl₂, 1.2 mmol/l MgSO₄, 1.2 mmol/l KH₂PO₄) containing 2.8 mmol/l glucose and 0.1% BSA. Islets were perifused for 10 min at a rate of 0.3 ml/min, followed by stimulation with 16.7 mmol/l glucose for 35 min. Fractions were collected every 1-3 min and insulin secreted into fractions and the corresponding islet lysate insulin content was quantified by radioimmunoassay (RIA).

Skeletal Muscle Subcellular Fractionation.

Hind limb skeletal muscle was sub fractionated into sarcolemmal/t-tubule plasma membrane and intracellular membrane components. Doc2b Tg and Wt littermate female mice (4-6 months old) were fasted for 16 h (18:00-10:00), injected intraperitoneally with 21 U/kg body weight of Humulin or saline, and sacrificed within 40 min for removal of the hindquarter muscles into homogenization buffer (20 mmol/l Hepes pH 7.4, 250 mmol/l Sucrose, 1 mmol/1 EDTA, 5 mmol/l benzamidine, 10 μg/ml aprotinin, 5 μg/ml leupeptin, 1 μg/ml pepstatin, 1 mmol/l PMSF) for Polytron homogenization. Homogenates were centrifuged at 2000×g for 5 min at 4° C., and supernatant then centrifuged at 9000×g for 20 min at 4° C. That supernatant was subsequently centrifuged at 180,000×g for 90 min. Pellets containing t-tubule and sarcolemmal membrane fractions were resuspended in 1% NP-40 lysis buffer (25 mmol/l Tris, pH 7.4, 1% NP40, 10% glycerol, 50 mmol/1 sodium fluoride, 10 mmol/l sodium pyrophosphate, 137 mmol/l sodium chloride, 1 mmol/l sodium vanadate, 1 mmol/l PMSF, 10 μg/ml aprotinin, 1 μg/ml pepstatin and 5 μg/ml leupeptin) and proteins resolved by 10% SDS-PAGE for subsequent immunoblotting for GLUT4, Syn4, Munc18c and Doc2b.

L6 Muscle Cell Culture.

Rat L6 muscle cells stably expressing GLUT4 with an exofacial myc-epitope were obtained from Dr. Amira Klip (Department of Biochemistry, University of Toronto, Canada). Myoblasts were maintained in α-MEM containing 5.5 mmol/l glucose (Life Technologies, Gaithersburg, Md., U.S.A) and 10% fetal bovine serum (Fisher Scientific, Pittsburgh, Pa., U.S.A.), and 1% (v/v) antimycotic antibiotic solution (Life Technologies, Gaithersburg, Md., U.S.A.). Myoblasts at 80-90% confluence were electroporated (0.20 kV and 960 μFarad) with 150 μg of GFP vector or GFP-DOC2B plasmid DNA per 10 cm² dish. After electroporation, cells were allowed to adhere to plates for 48 hours. Cells were pre-incubated in serum-free media for 2 h followed by stimulation for 5 min with 100 nmol/l insulin. Cells were harvested in 1% NP-40 lysis buffer and detergent lysates used for co-immunoprecipitation or GST-VAMP2 interaction assays.

CHO-K1 Cell Culture.

CHO-K1 cells were cultured in Ham's F-12 medium supplemented with 10% fetal bovine serum, 100 units/ml penicillin, 100 μg/ml streptomycin, and 292 μg/ml L-glutamine. At 80-90% confluence, cells were electroporated with 40 μg of plasmid DNAs: pN2-GFP-DOC2B or pN2-GFP-DOC2A, per 10 cm² dish. After 48 h of incubation, cells were harvested in 1% NP-40 lysis buffer and detergent lysates were prepared and proteins resolved by 10% SDS-PAGE for immunoblotting for Doc2b and GFP. GFP-Doc2a and -Doc2b fusion proteins migrate at ˜73 and 75 kDa, respectively (pN2-eGFP encodes GFP of 29 kDa, Doc2b migrates at ˜46 kDa, Doc2a at ˜44 kDa).

Co-Immunoprecipitation.

Cleared detergent lysates from L6 myoblasts (2.5 mg) were combined with rabbit anti-Syn4 antibody for 2 h at 4° C., followed by a second incubation with protein G Plus-agarose for 2 h. The resultant immunoprecipitates were subjected to 10-12% SDS-PAGE followed by transfer to PVDF membranes for immunoblotting.

Recombinant Proteins and Interaction Assays.

The GST-VAMP2 protein was generated in E. coli and purified by glutathione-agarose affinity chromatography for use in the Syntaxin 4 accessibility assay. GST-VAMP2 protein linked to Sepharose beads was combined with 2 mg of L6 myoblast detergent cell lysate for 2 h at 4° C. in 1% NP-40 lysis buffer, followed by three stringent washes with the lysis buffer. The associated proteins were resolved on 10% SDS-PAGE followed by their transfer to PVDF membranes for immunoblotting for Syn4.

Statistical Analysis.

All data were evaluated for statistical significance using student t-test for comparison of two groups; ANOVA was used otherwise. Data are expressed as the average±standard error (SE).

### Example VI

### Generation of Doc2b Transgenic Mice

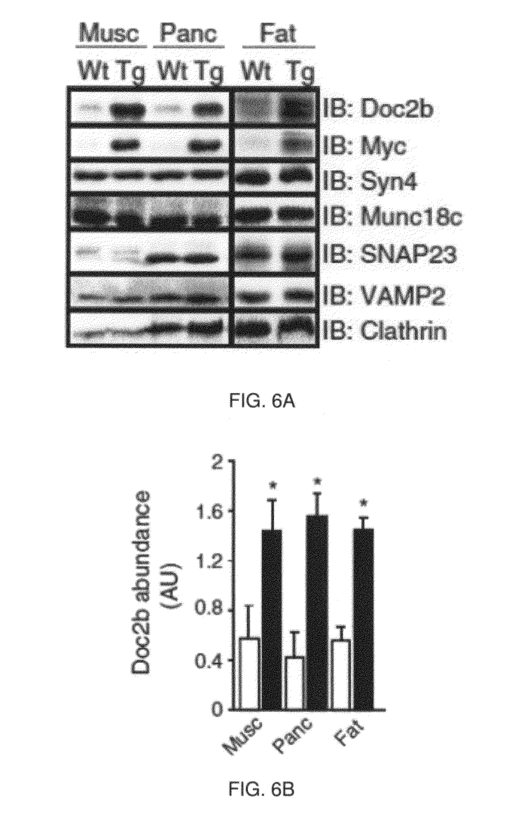

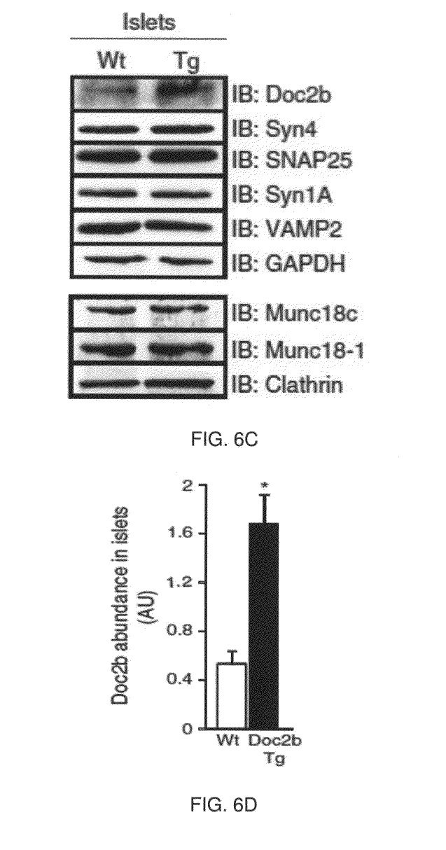

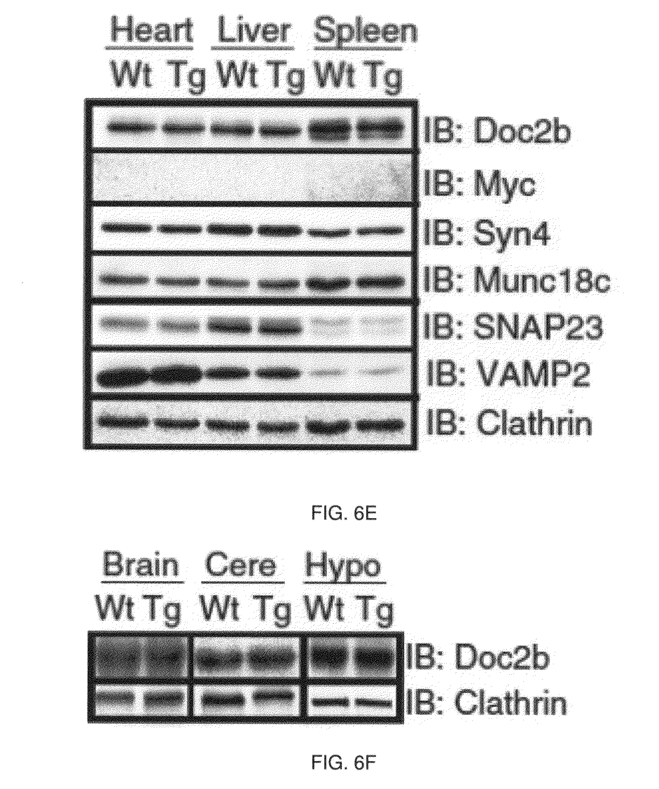

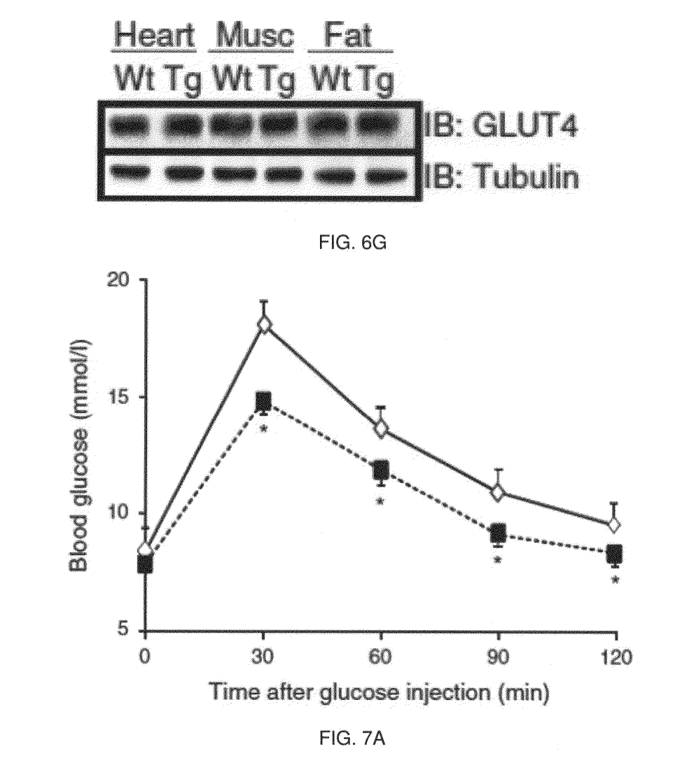

Tetracycline-repressible Doc2b Tg mice were generated using a targeting vector previously shown to drive expression in almost exclusively in pancreas, skeletal muscle, and adipose tissue described in Spurlin et al., Diabetes 2004, 53:2223-2231 and Spurlin et al Diabetes, 2003, 52:1910-1917. Of the four founder lines, three transmitted the DOC2B gene, and one line consistently exhibited about 2-3-fold increases in Doc2b protein relative to endogenous expression in Wt littermates in these tissues (FIGS. 6A-6B). No alterations in the levels of SNARE proteins such as Syn4, SNAP23, VAMP2 and Munc18c were detected. Doc2b protein levels in Tg islets were increased about 3 fold over that in Wt islets, again with no alteration in expression of SNARE or Munc18 proteins (FIGS. 6C-6D). No transgene expression was detected in heart, liver or spleen (FIG. 6E), nor in whole brain lysate, cerebellum or hypothalamus (FIG. 6F), similar to other reports using this targeting vector. GLUT4 protein levels in heart, skeletal muscle and fat of Doc2b Tg mice were similar to that in Wt mice (FIG. 6G).

### Example VII

### Improved Glucose Tolerance in Doc2b Enriched Mice

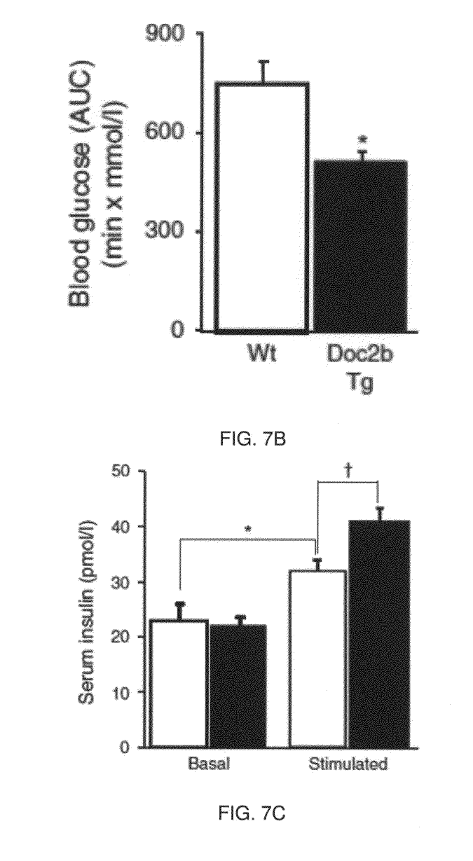

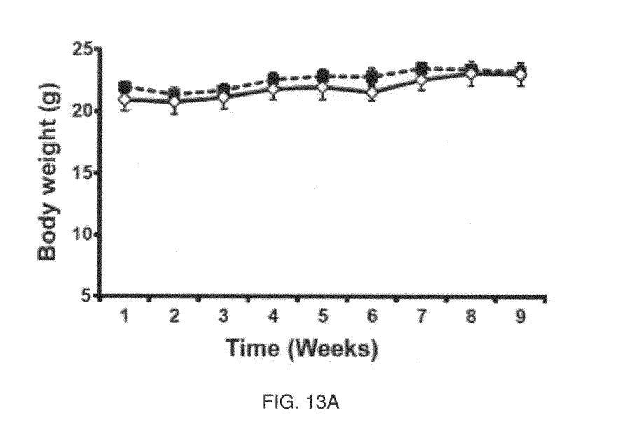

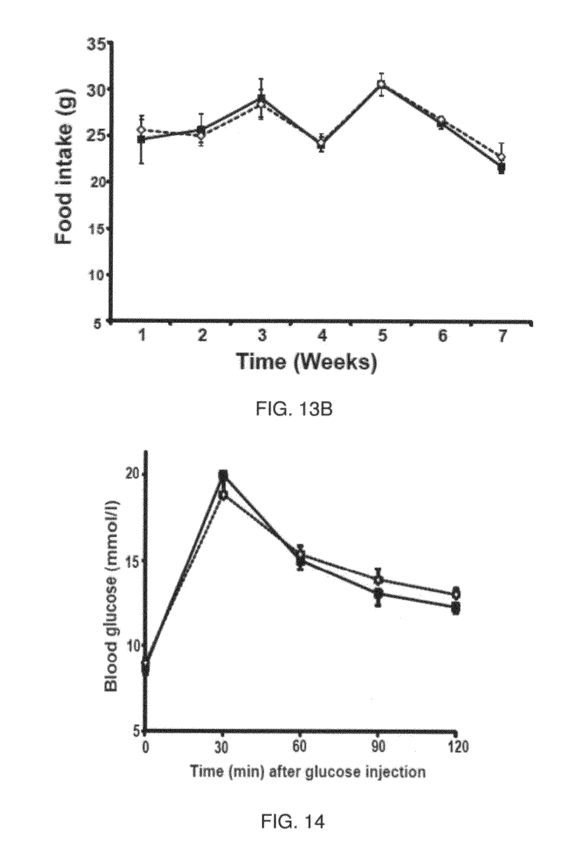

To determine whether Doc2b expression in these select tissues was limiting for glucose homeostasis in vivo, intraperitoneal glucose tolerance tests (IPGTT) were performed. While fasting glycaemia was unaltered, Doc2b Tg mice showed significantly lower blood glucose levels than Wt mice upon glucose challenge at all time points (FIG. 7A). Area under the curve (AUC) analysis quantified this to be a 44% improvement in glucose tolerance (FIG. 7B). This improved glucose clearance correlated with significantly increased serum insulin content in the Doc2b Tg mice within the first 10 minutes after glucose injection during the IPGTT (FIG. 7C). Body weights and food intake of Doc2b Tg mice were equivalent to that of Wt mice over 7-9 weeks time (FIGS. 13A-13B). The lower expressing transgenic line (<1.3-fold) was similar to Wt responsiveness in the IPGTT (FIG. 14). These data indicate that about 2-3-fold enrichment of Doc2b simultaneously in skeletal muscle, fat and pancreas was sufficient to enhance glucose tolerance via heightened insulin release and/or peripheral glucose uptake.

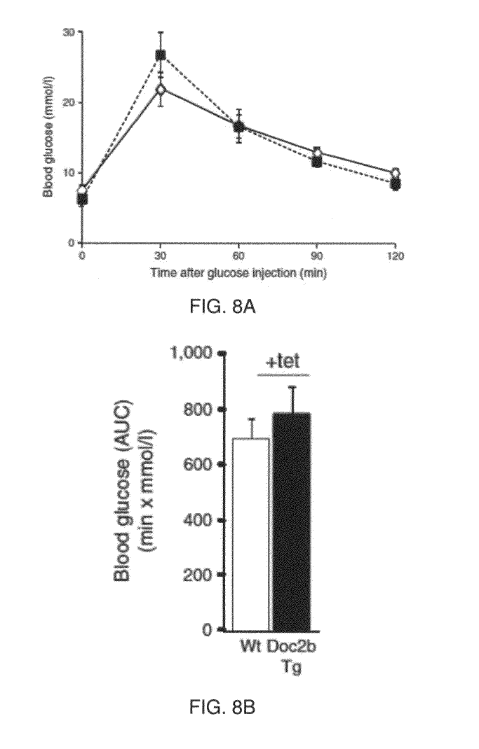

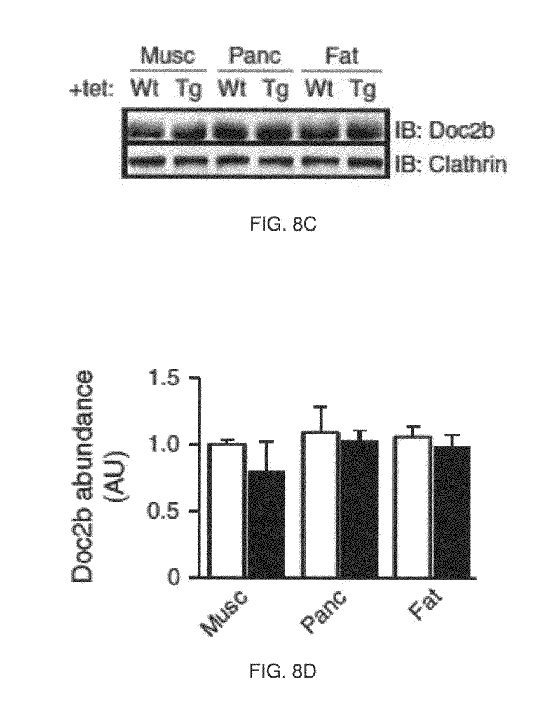

To validate that the enhanced glucose tolerance of the Doc2b Tg mice was due to the presence of the transgene, the same Doc2b Tg and Wt mice examined by IPGTT in FIG. 7A were administered tetracycline (tet)-treated drinking water for 1 week to repress the transgene, after which the IPGTT was repeated. Glucose tolerance of the tet-treated Doc2b Tg mice was similar to that of Wt mice (FIGS. 8A-8B), as were the Doc2b protein levels (FIGS. 8C-8D). These data confirmed that the enhanced glucose tolerant phenotype of the Doc2b Tg mice corresponded to the expression of the DOC2B transgene.

### Example VIII

### Doc2b Enrichment Potentiates Biphasic GSIS

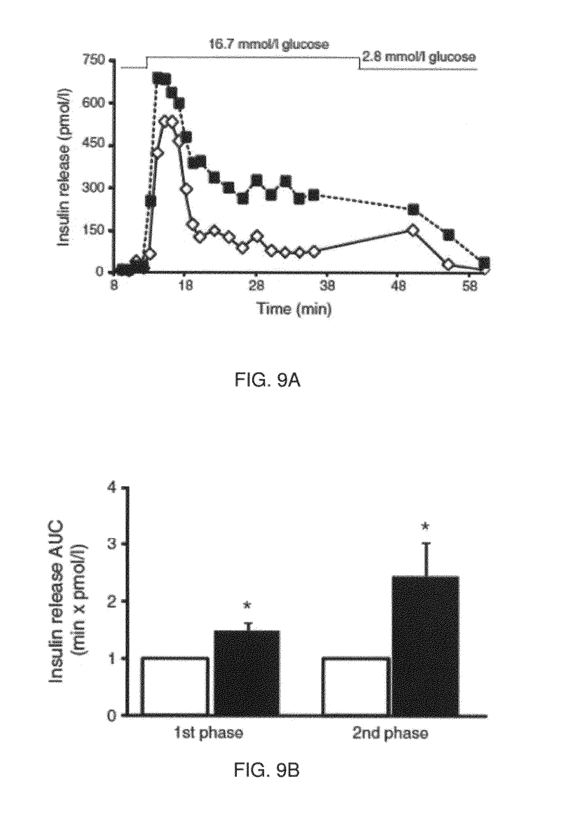

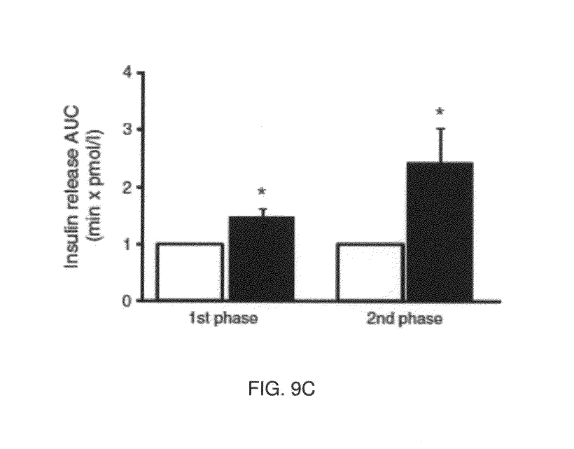

To determine if the increased serum insulin content of Doc2b Tg mice was related to increased islet function, islet cells were subjected to parallel perifusion analyses. Islets of Doc2b Tg mice exhibited a higher peak of first-phase insulin release as well as sustained elevation of second-phase release (FIG. 9A). AUC analysis of Doc2b Tg secretion revealed a 50% increase over that of Wt islets in the first phase, and a 250% increase in second phase relative to Wt islets (FIG. 9B). Total insulin content was similar between Doc2b Tg and Wt islets (FIG. 9C). Basal insulin secretion was similar between Doc2b Tg and Wt islets (FIG. 9A), consistent with similar fasting serum insulin contents of the mice (FIG. 7C). These data indicate Doc2b to be limiting for each phase of GSIS and that its enrichment could enhance functional GSIS without aberrantly raising basal insulin release.

### Example IX

### Doc2b Tg Mice have Enhanced Insulin Sensitivity and Cell Surface GLUT4 Accumulation in Skeletal Muscle

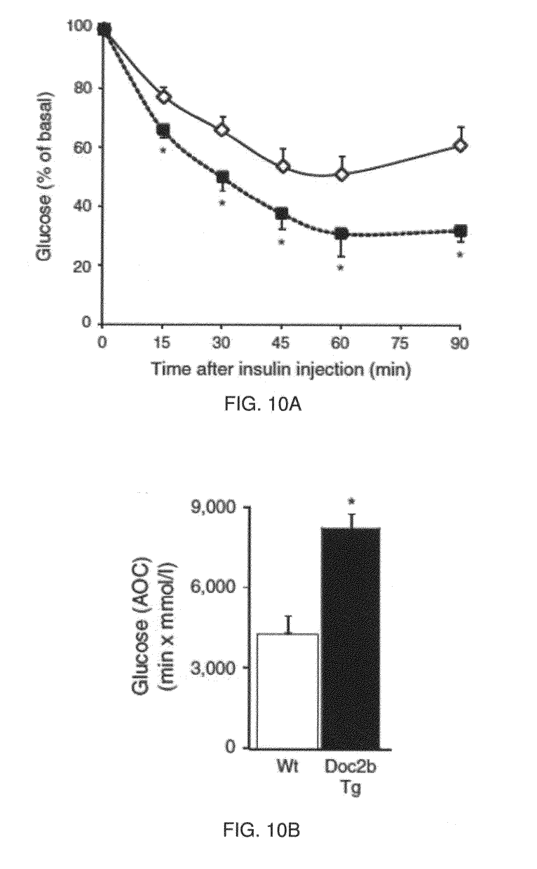

It was next assessed whether the beneficial effect of Doc2b enrichment on glucose tolerance was related to improved peripheral insulin sensitivity, by performing intraperitoneal insulin tolerance tests (ITT). Following insulin injection (0.75 U/kg body weight), blood glucose levels of the littermate WT mice dropped by about 45% within 60 min, as is normal for the C57BL/6J strain (FIG. 10A). In Doc2b Tg mice, both the rate and extent of the reduction in glycaemia was significantly enhanced (FIG. 10A). Area over the ITT curve for the Doc2b Tg mice showed this to be a nearly 2-fold improvement in glycaemia in the Doc2b Tg mice compared to Wt mice (FIG. 10B).

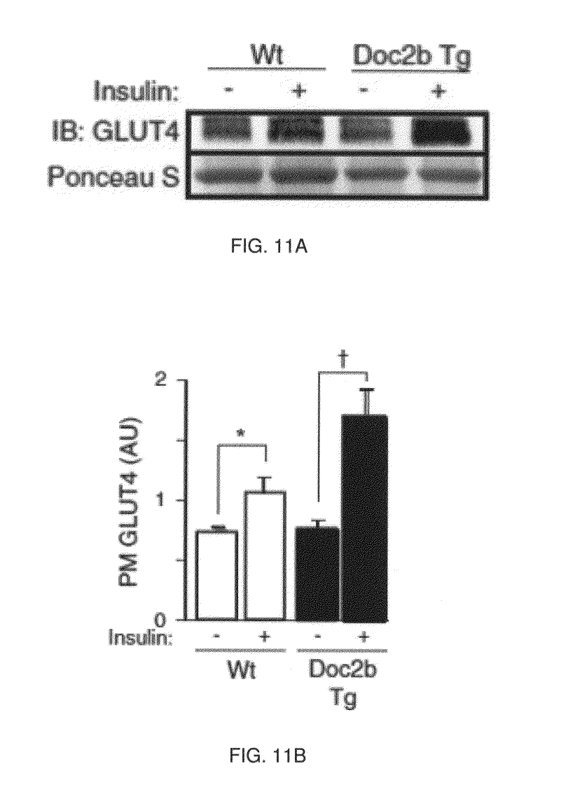

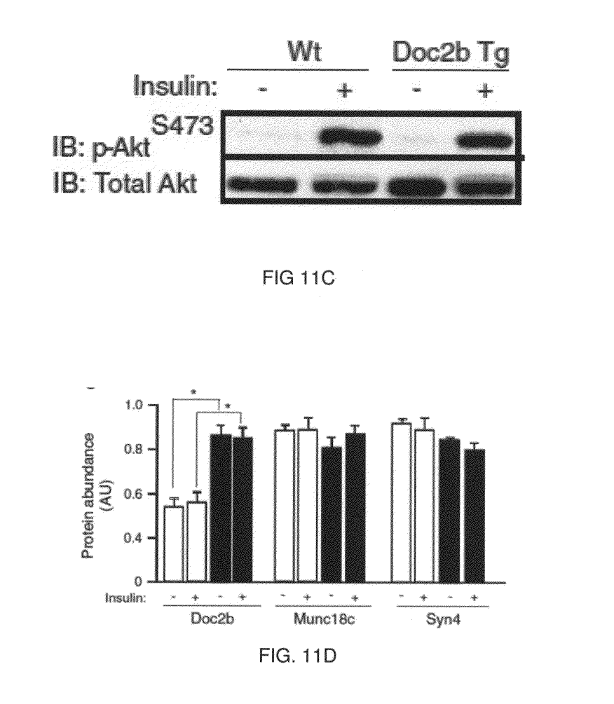

The improved insulin sensitivity indicated that Doc2b is limiting for insulin signalling or insulin-stimulated GLUT4 vesicle translocation to the cell surface membranes in skeletal muscle, given that skeletal muscle accounts for about 80% of glucose uptake in humans. To verify this, t-tubule/sarcolemmal cell surface enriched membrane fractions (referred to as PM) were isolated from saline- or insulin-injected hind limb skeletal muscle of Doc2b Tg and Wt mice. PM fractions prepared from insulin-injected Wt mice showed the expected about 1.5-fold increase in GLUT4 accumulation in insulin-stimulated versus the unstimulated Wt mouse muscles. Doc2b Tg PM fractions exhibited about 2.2-fold increase in insulin-stimulated GLUT4 vesicle accumulation at the PM (FIGS. 11A-11B). Since Akt activation in Doc2b Tg skeletal muscle homogenates was similar to that of Wt mice (FIG. 11C), the beneficial action of Doc2b enrichment appeared to lie downstream of Akt activation. Toward this, SNARE and Munc18 abundances were assessed for differences. While Doc2b levels were elevated in the Tg PM muscle fractions compared with Wt, Syn4 and Munc18c protein levels were similar between Doc2b Tg and Wt muscle PM fractions, under both unstimulated and insulin-stimulated conditions (FIG. 11D). Collectively, these data indicate the enhanced insulin-stimulated GLUT4 vesicle accumulation (docking/fusion) at the PM is an underlying cause for the enhanced peripheral insulin sensitivity of the Doc2b Tg mice.

### Example X

### Doc2b Enrichment Promotes SNARE Complex Formation in Skeletal Myoblasts

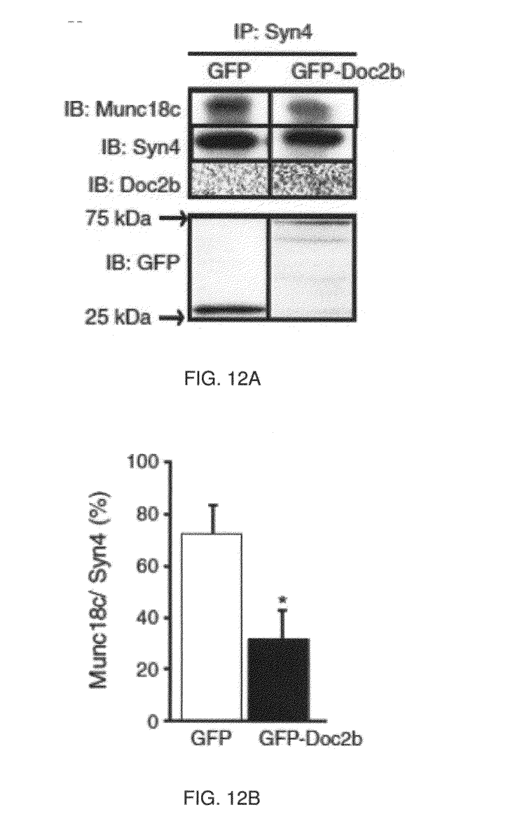

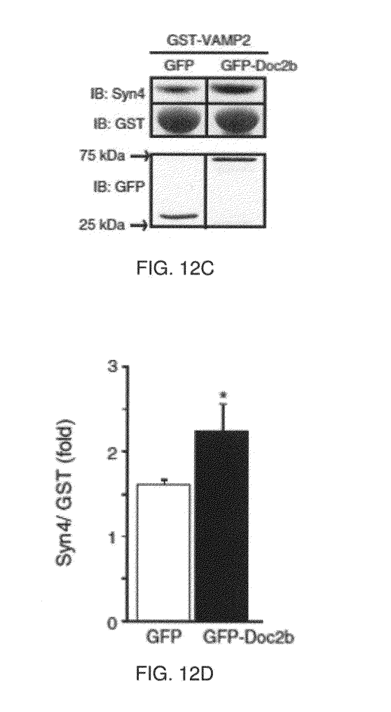

To determine how Doc2b enrichment in skeletal muscle of the Doc2b Tg mice might increase the PM-localization of GLUT4, Munc18c-Syn4 binding and Syn4 activation/SNARE complex formation was studied in L6-GLUT4-myc myoblasts transfected to express exogenous GFP-tagged Doc2b (or GFP vector control). L6-GLUT4-myc skeletal myoblasts are the premiere clonal muscle cell line for recapitulating the events associated with GLUT4 vesicle exocytosis and transfect with ˜30-50% efficiency. Immunoprecipitation of Syn4 from insulin-stimulated GFP-Doc2b-expressing L6 cells showed significantly reduced Munc18c co-precipitation compared to GFP-expressing cells (FIGS. 12A-12B). Syn4 accessibility to VAMP2 was examined as an indicator of its ability to form SNARE complexes, given the inability to obtain sufficient PM protein from transfected L6 cells for co-immunoprecipitation analyses. Syn4 accessibility to an exogenous GST-VAMP2 protein linked to sepharose beads from insulin-stimulated GFP-Doc2b expressing L6 myoblasts was significantly enhanced relative to cells expressing a similar amount of GFP (FIG. 7B).

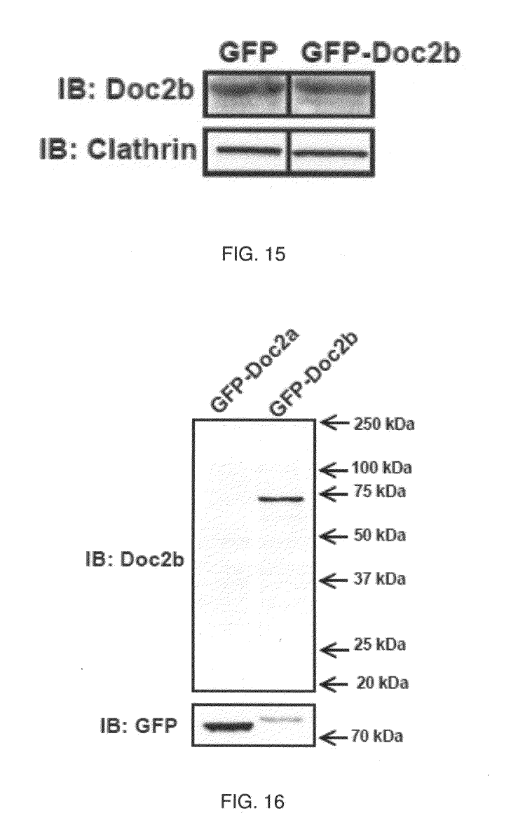

Endogenous Doc2b levels were similar amongst transfected cells, indicating that the enrichment in SNARE complex formation was due to the effects of overexpressed Doc2b (FIG. 15). The Doc2b antibody used for this and all Doc2b immunoblotting was confirmed to react specifically with Doc2b (FIG. 16). Together, these results indicate that Doc2b is a limiting factor for Munc18c-Syn4 dissociation in skeletal muscle cells, and that the potentiation of Syn4 accessibility by increased expression of Doc2b may underlie the enhanced GLUT4 accumulation in PM fractions and insulin sensitivity of the Doc2b Tg mice.

### Example XI

### Increased Longevity in the Insulin-Sensitive Syntaxin 4 Transgenic Mouse—Materials and Methods

Animals.

All animal studies were approved by the Indiana University School of Medicine Institutional Animal Care and Use Committee. The rat Syntaxin 4 cDNA inserted into the pUC-Combi targeting vector (Schultze et al., 1996) was used to generate heterozygous transgenic mice on the C57BL/6J strain background as described (Spurlin et al., 2004); wildtype (Con) littermates served as controls. All physiological aging, metabolic and feeding studies used three cohorts each, randomizing for potential variations in external environment (season and chow components), parentage (sire/dam pairing) and intrauterine environment.

Immunoblotting.

The following antibodies were used: mTOR (2972, Cell Signaling), p-mTOR (Ser 2448) (2971, Cell Signaling), SirT1 (D739) (2493, Cell Signaling), FoxO1 (C29H4) (2880 Cell signaling), p-FoxO1 (Ser256) (9461, cell Signaling), VAMP2 (104211, Synaptic Systems, Germany), SNAP 23 (PA1-738, Affinity Bioreagents), Tubulin (T5168 Sigma). Rabbit anti-Syn4 and Munc18c antibodies were generated in-house as described (Thurmond et al., 1998; Wiseman et al., 2011). Goat-anti-rabbit-horseradish peroxidase and anti-mouse-horseradish peroxidase secondary antibodies were purchased from Bio-Rad (Hercules, Calif., USA).

Metabolic Studies.

For IPGTT and ITT studies, littermate Con and Syn4 Tg mice were fasted for 16 h and 6 h respectively. Following sample collection of fasted blood, animals were injected intraperitoneally with glucose (2 g/kg body weight) or insulin (0.75 U/kg body weight Humulin R; Eli Lilly and Company, Indianapolis Ind.), respectively, and blood glucose sampled from the tail vein every 15-30 min using the Hemocue glucometer (Mission Viejo, Calif.). To measure serum metabolite levels, littermate Con and Syn4 Tg mice were fasted for 16 h and serum samples were submitted to Vanderbilt Hormone Assay Core facility. For Glucagon secretion assay, littermate Con and Syn4 Tg mice were fasted for 6 h and animals were injected intraperitoneally with glucose (2 g/kg body weight), then blood from the tail vein was collected at 10 min. Serum was analyzed using the glucagon RIA immunoassay kit from Millipore. Indirect calorimetric measurement was performed using a TSE systems LabMaster Metabolism Research Platform (Chesterfield, Mo.) equipped with calorimeter, feeding and activity system. Con and Syn4 Tg mice (12 months old) were acclimated for 48 h and measurements of food intake, movement and respiratory quotient (RQ) during 24 h were collected every 10 min during light (07:00-19:00) and dark cycles (19:00-07:00). Body fat percentage was measured by dualenergy X-ray absorptiometry (DEXA) using a Lunar PIXIMus DEXA Scanner (GE Medical Systems, Fairfield, Conn.) on Con and Syn4 Tg mice (8 months old) with inhalational anesthesia (isofluorane). Serum analytes other than insulin were quantified by the Hormone Assay and Analytical Services Core at Vanderbilt University (Nashville, Tenn.).

Diet Composition and Feeding.

Male mice at 12 weeks of age were individually housed for the 10 week high-fat diet (HFD) study. The study was conducted in three separate cohorts with 2 or more mice per feeding group (Con-HFD, Syn4 Tg HFD). Each cohort was conducted at a different time over the span of 9 months in an effort to obtain randomized results from independent litters of mice. Body weight and food intake measurements were taken daily along with the replenishment of fresh diet. The proximate profile of the HFD (cat. #F3282, Bio-Serv, Frenchtown N.J.) was 20% protein, 35.5% fat, 32.7% carbohydrate, 3.7% ash, 0.1% fiber and <10% moisture. Fatty acid composition was 161 g/kg oleic, 89 g/kg palmitic, 44 g/kg stearic, 35 g/kg linoleic, 11 g/kg cis-9-hexadecanoic, 5 g/kg myristic, 4.7 g/kg cis-11-eicosenoic, with all others at 1.5 g/kg or less. Carbohydrates consisted of 195 g/kg polysaccharides, 149 g/kg disaccharides, 13 g/kg trisaccharides and no monosaccharides. The caloric profile of the HFD was 16% protein, 59% fat and 25% carbohydrate. The caloric profile of the regular diet (RD, cat. #7012 Teklad, Harlan, Indianapolis Ind.) was 22% protein, 11% fat and 60% carbohydrate with 3.41 kcal/g metabolizable energy.

Skeletal Muscle GLUT4 Translocation.

Littermate male mice (4-6 months old) were fasted for 16 h, injected intraperitoneally with Humulin or vehicle (saline) at 21 U/kg body weight for 40 min, and sacrificed for removal of the hindquarter muscles for Polytron homogenization and subsequent differential centrifugation to yield the P2 fractions (contain transverse tubule and sarcolemmal membranes) and intracellular membrane components as described (Spurlin et al., 2004; Zhou et al., 1998). P2 fractions proteins were solubilized in 1% NP40 lysis buffer and proteins resolved on 10-12% SDS-PAGE for immunoblotting for GLUT4 (Santa Cruz)

Pancreatic Immunohistochemistry and Islet Immunofluorescence.

β cell areas in Syn4 Tg and littermate Con mice were determined as described (Wang et al., 2011), wherein pancreata from five-month old male Con or Syntaxin 4 Tg mice were fixed with 10% formalin (neutralized buffer), paraffin embedded, and longitudinally sectioned at 5-μm thickness and 100-μm intervals for insulin staining and counterstaining with hematoxylin. Digital images were acquired on an Axio-Observer Z1 microscope (Zeiss) fitted with an AxioCam high resolution color camera and associated software used to calculate beta cell area. Data shown are representative of 3-4 sections per pancreas and 3 pancreata from each group. For immunofluorescence staining, slides were incubated with insulin antibody (Invitrogen, Carlsbad, Calif.), and glucagon antibody (Sigma, St. Louis, Mo.) and followed by incubation with Alexa Fluor 488- or Alexa 555-conjugated secondary antibodies (Invitrogen). Images were recorded on the laser-scanning confocal microscope (FV1000, Olympus) using Fluoview software (FV10-ASW version 1.7).

Isolation, Culture and Stimulation of Insulin Secretion of Mouse Islets.

As described (Spurlin et al., 2004), islets were isolated from pancreata of Syn4 Tg and Con mice at 6 months of age (FIG. 3D-E) or 22 weeks (FIG. 4D) and were cultured overnight in CMRL-1066 medium. Fresh islets were hand-picked into groups of 10, pre-incubated in Krebs-Ringer bicarbonate buffer (10 mM HEPES pH 7.4, 134 mM NaCl, 5 mM NaHCO3, 4.8 mM KCl, 1 mM CaCl2, 1.2 mM MgSO4, 1.2 mM KH2PO4) containing 2.8 mM glucose and 0.1% BSA for 2 h, followed by stimulation with 20 mM glucose for 2 h. Media was collected to measure insulin secretion and islets were harvested in NP-40 lysis buffer to determine cellular insulin content by radioimmunoassay.

Microarray.

Hindlimb skeletal muscle was dissected from Con and Syn4 Tg mice and immediately frozen in liquid nitrogen to preserve RNA quality. Total RNA from Con or Syn4 Tg was labeled and hybridized to Affymetrix Mouse Genome GeneChips. The array was analyzed by the Indiana University School of Medicine Center for Medical Genomics (Indianapolis, Ind.).

Statistical Analysis.

All values are presented as means±SE. Differences were analyzed by Student's t test or two-way ANOVA.

### Example XII

### Relationship Between Syn4 Abundance and Aging

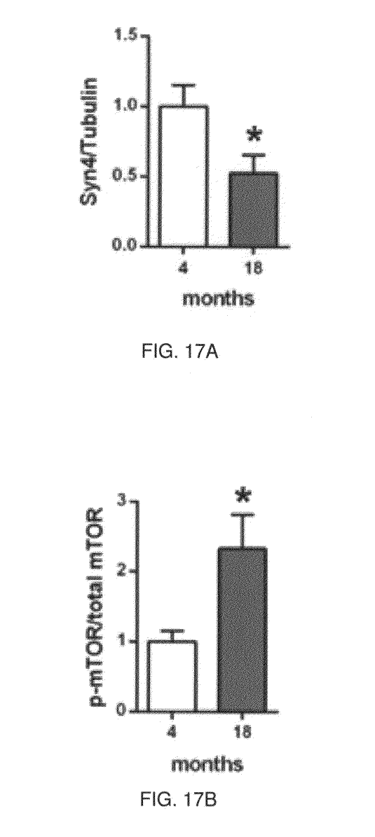

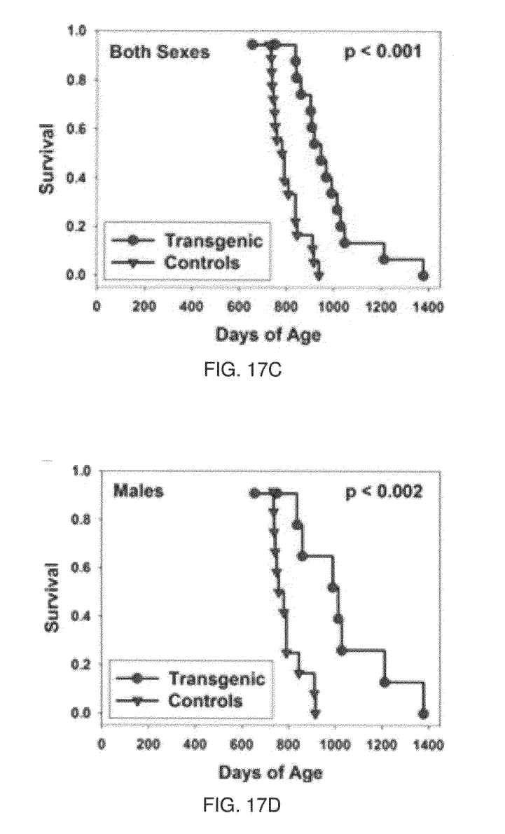

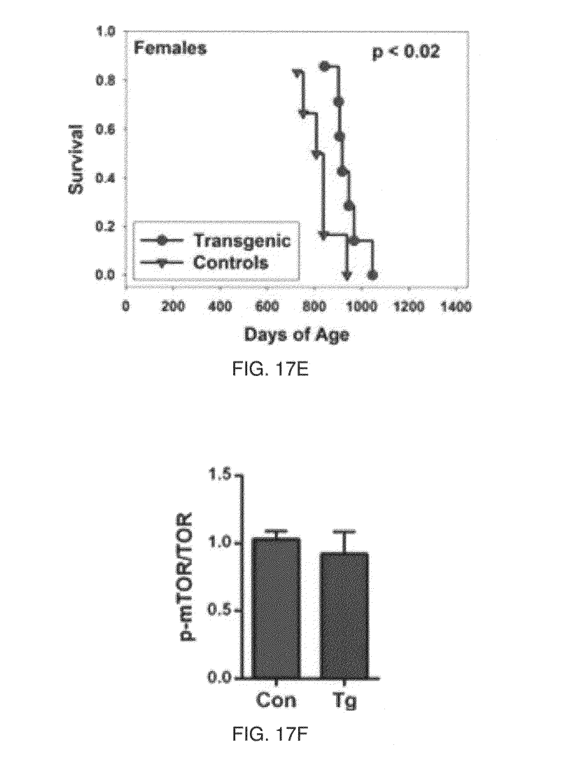

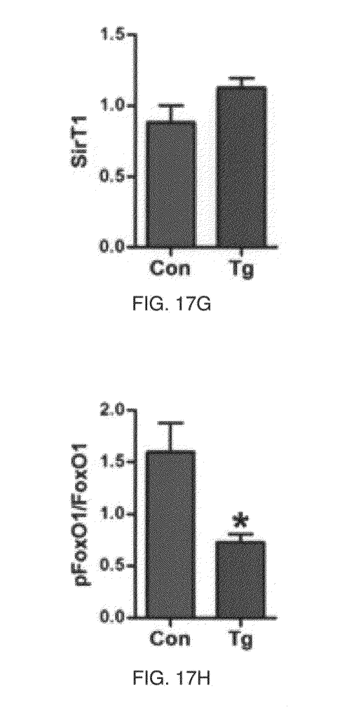

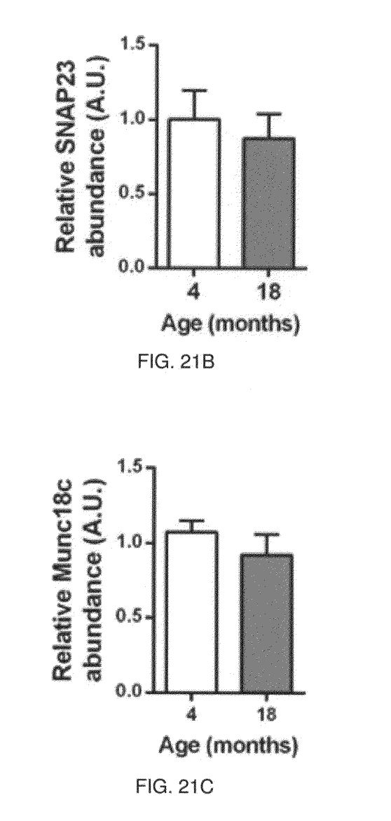

The abundances of exocytosis proteins in pancreas lysates of differentially aged mice were investigated. Syn4 was found to be attenuated in a manner inversely related to that of the known aging-induced p-mTOR (FIGS. 17A-17B). No differences in pancreatic abundances of Syn4-based SNARE partners VAMP2, SNAP23 or Munc18c were observed with aging (FIGS. 21A-21C). This observation led to a longitudinal study of Syn4 transgenic (Syn4 Tg) mice. These mice express 3-5 fold more Syn4 protein specifically in the pancreas, skeletal muscle and adipose tissues, and exhibit enhanced insulin sensitivity, accelerated glucose clearance, and have the capacity to release ˜30% more insulin per islet while retaining biphasic kinetics.

The Syn4 Tg mice lived ˜33% longer than wild type control (Con) littermates (p<0.0001), with similar effects in male and female mice (FIGS. 17C-17E). In this regard, up-regulation of Syn4 yields lifespan extension similar to that produced by caloric restriction, and more than rapamycin or acarbose treatments (Harrison et al., 2014; Miller et al., 2014). Aging-related gene products such as SIRT1 or mTOR were unchanged in pancreata of Syn4 Tg mice relative to littermate controls (FIGS. 17F-17G). While the amount of phosphorylated FoxO1 (Ser256) was also unchanged in Syn4 Tg pancreata, total FoxO1 levels were 2-fold greater, thus decreasing the relative level of phosphorylated FoxO1 (FIG. 1H). Microarray data from skeletal muscle of Syn4 Tg mice otherwise showed no changes in other genes implicated in aging (Table 3). These data collectively show the existence of a specific inverse relationship between aging and Syn4 abundance.

TABLE 3
Microarray data from Syn4 Tg and Control mouse skeletal muscle

| | Fold: Syn4 Tg/Con |
|---|---|
| Sirtuin 1 | −1.0 |
| FoxO1 | 1.06 |
| MTOR | 1.03 |
| Igf1r | 1.07 |
| Foxo3a | 1.05 |
| ApoC3 | −1.04 |
| Tshr | 1.23 |
| Ghr | 1.0 |
| Ir | 1.02 |
| Sirtuin 6 | −1.01 |
| Mif | −1.02 |
| Sirtuin 3 | −1.0 |
| Sirtuin 4 | 1.0 |
| Irs1 | 1.02 |
| Irs2 | −1.02 |

Note:
PEPCK-C, AMPK, CEPT, AdipQ, human TDP-43, PARP-1, 4E-BP and Sty1 MAP kinase transcripts were not detected in the microarray studies. Con, control.

### Example XIII

### Glucose Homeostatic Mechanisms in Long-Lived Syn4 Tg Mice

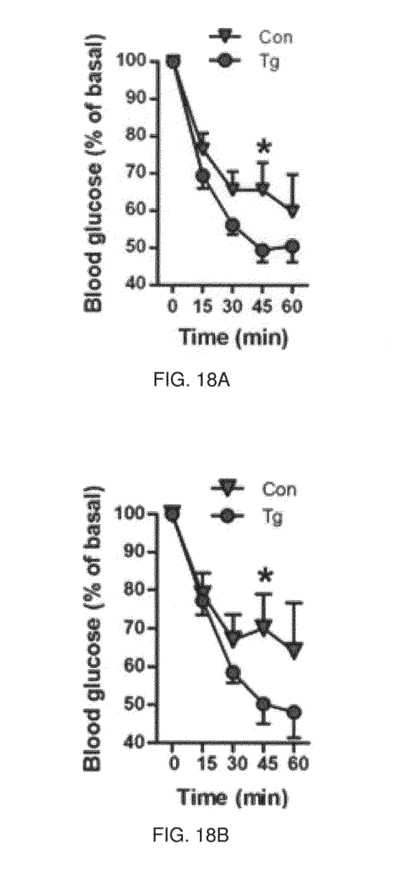

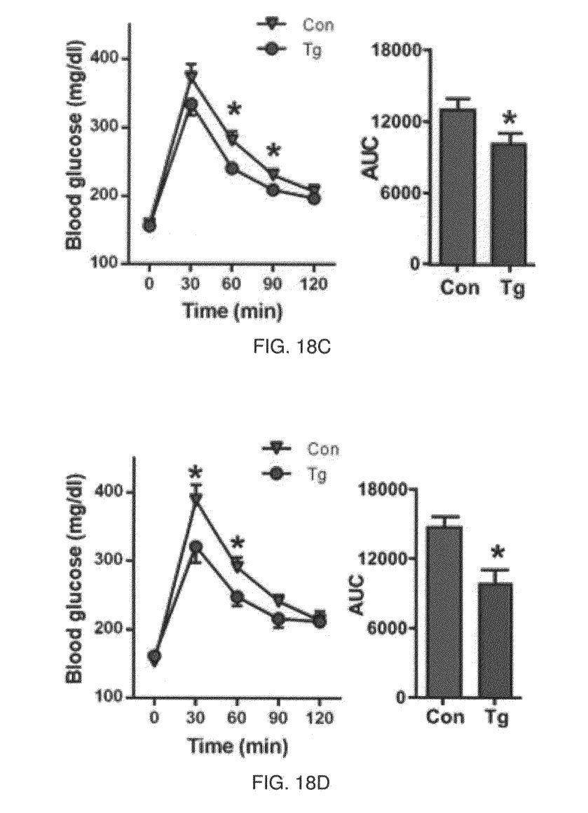

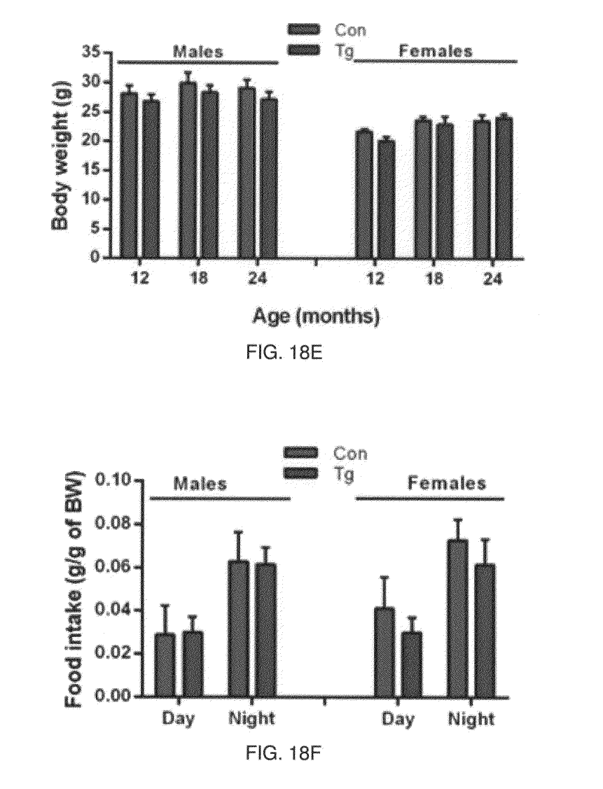

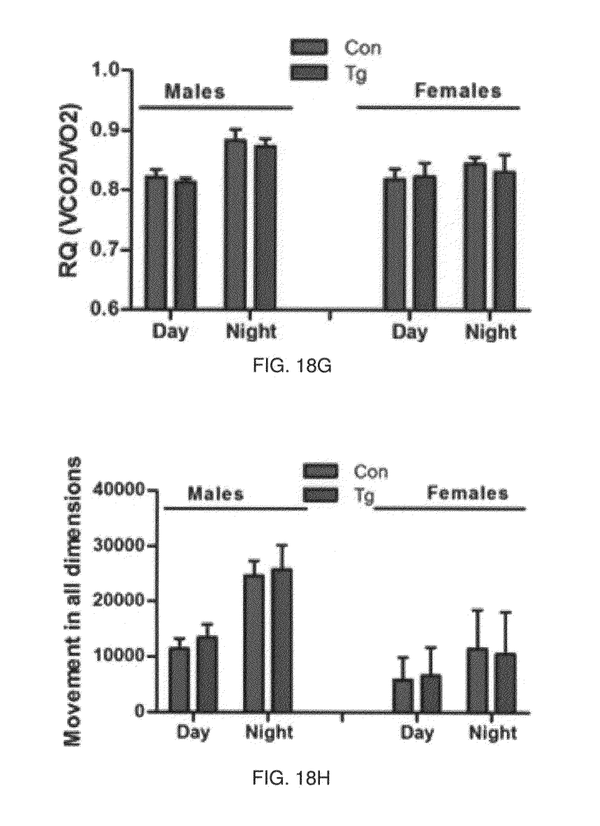

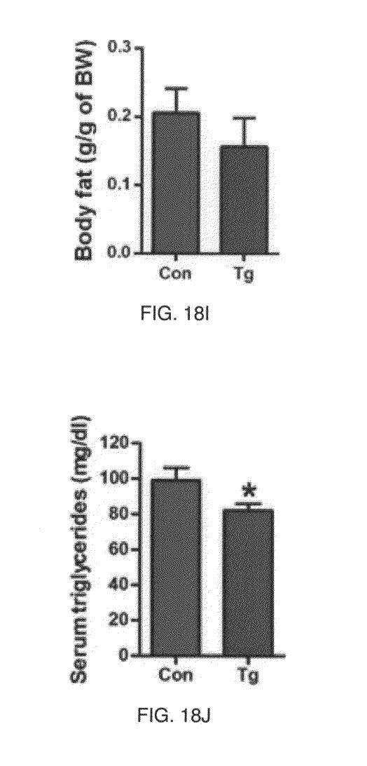

Comparing the insulin tolerance tests of month-old mice (n=25) with the response of this strain at 4-6 months of age, the Syn4 Tg mice showed enhanced insulin sensitivity (FIG. 18A). The males alone showed significant sensitivity (FIG. 18B), notable, as aged C57BL/6J males normally show insulin resistance. Consistent with this result, aged Syn4 Tg mice were significantly more glucose tolerant than controls (FIGS. 18C-D). Syn4 upregulation was not associated with dwarfism and occurred independent of caloric restriction as evidenced by body weight and food intake measures (FIGS. 18E-2F). Metabolic caging studies showed no significant differences between 12 month old Con and Tg mice for respiratory quotient (RQ) and movement/activity level (FIGS. 18G-18H). Although body fat content was also similar (FIG. 18I), serum triglycerides were slightly but significantly diminished in the Syn4 Tg mice (FIG. 18J). These data show that increasing the capacity for exocytosis in skeletal muscle is sufficient to protect against age-induced insulin resistance

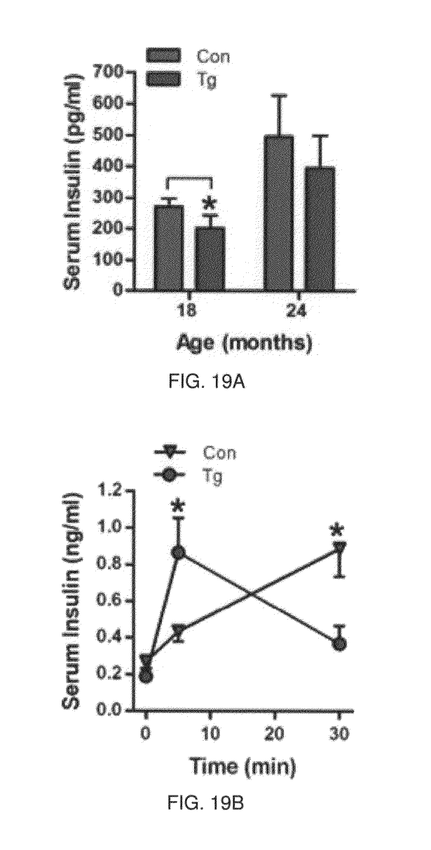

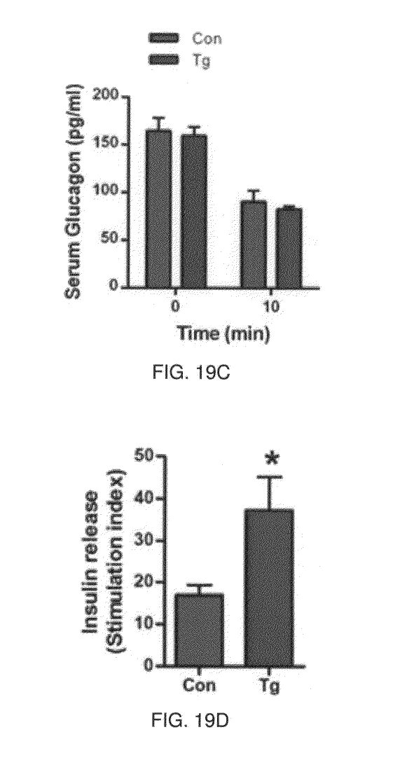

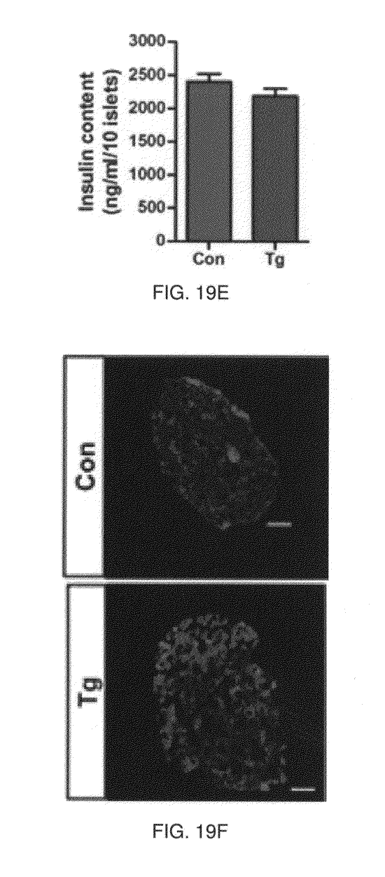

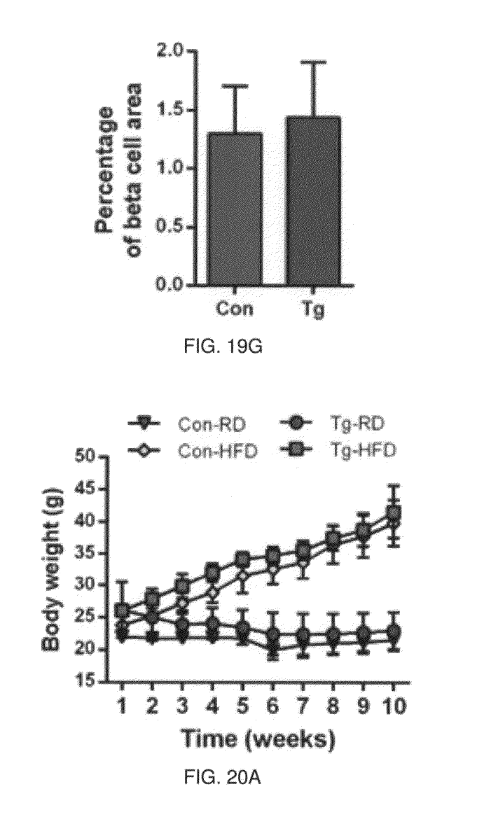

Glucose tolerance and homeostasis is substantially affected by functional β cell mass. Serum insulin content in fasted Syn4 Tg versus Con mice was reduced at 18 months of age (FIG. 19A), rising in both strains by age 24 months (P=0.0275 by two-way ANOVA). Following a glucose injection, 6 month old Syn4 Tg mice exhibited a left-shifted insulin response in vivo, showing a peak in serum insulin content by 5 min, as opposed to that seen in control mice (FIG. 19B); serum glucagon levels were not different between Syn4 Tg and Con mice under fasted or glucose-stimulated conditions (FIG. 19C). The rapid serum insulin response of the Syn4 Tg mice is consistent with an amplified glucose-stimulated insulin secretory response from their islets ex vivo (FIG. 19D), and was not affiliated with changes in islet insulin content or distribution of β and α cells within the islets (FIGS. 19E-19F). The percentage of β cell area remained similar between 18 month old Syn4 Tg and Con mice (FIG. 19G), although both were elevated by ˜2-fold relative to that reported for 6 month old mice (Oh et al., 2014). These data demonstrate that islet function was increased in the Syn4 Tg mice, independent of insulin content and islet morphometry.

### Example XIV

### Syn4-Induced Resistance to Obesity-Induced Dysfunction

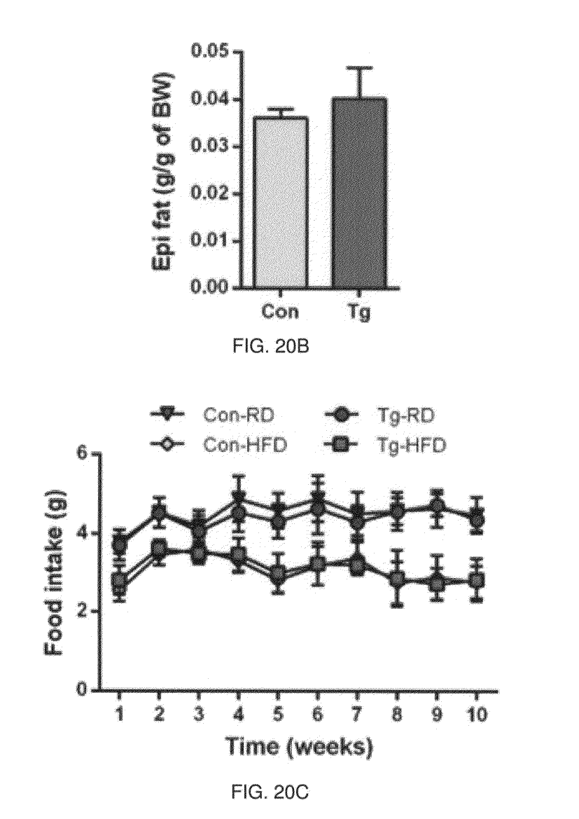

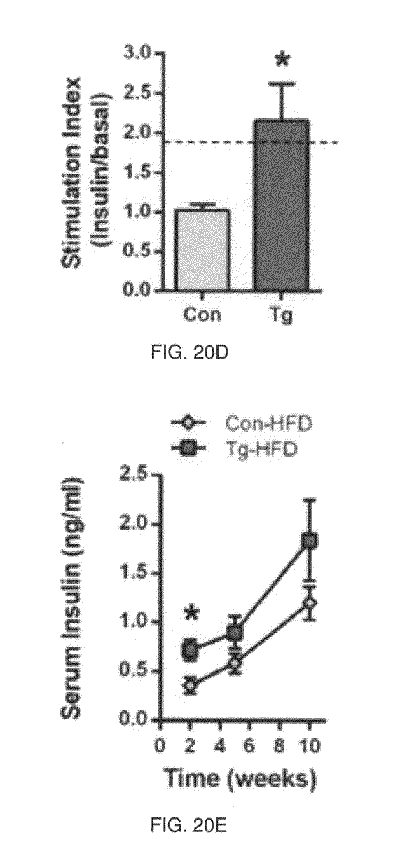

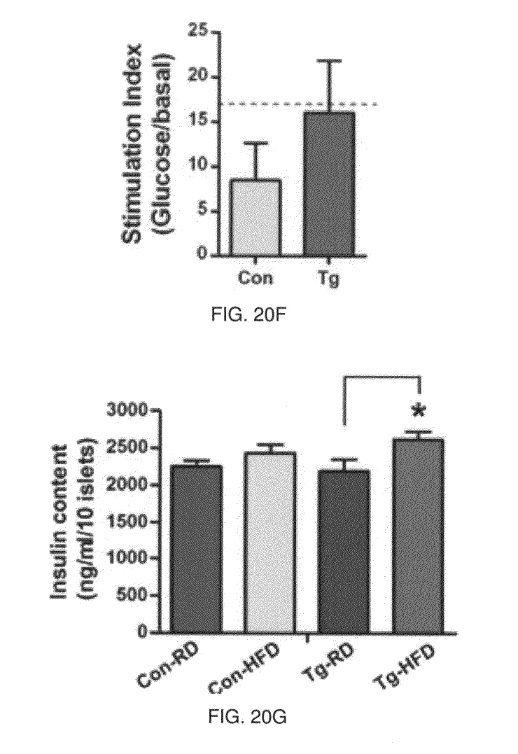

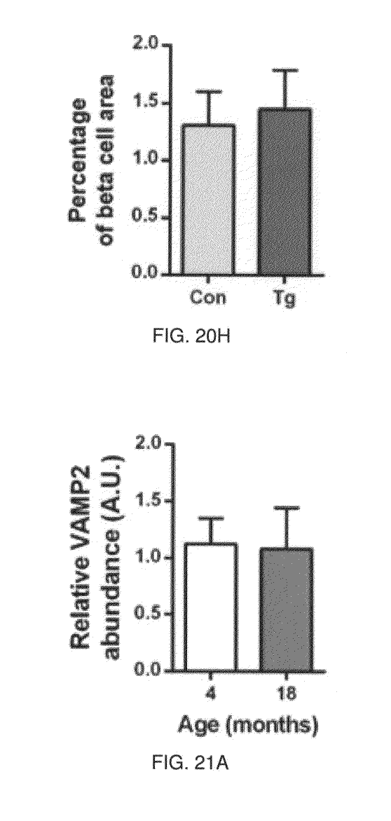

The onset of pre/type 2 diabetes with aging is associated with increasing adiposity/obesity in humans. Activities of proteins associated with lifespan and longevity, such as FoxO3, FoxO1, mTOR, IRS1, AMPK, and SIRT1 reportedly change with high fat diet (HFD). To assess whether Syn4 Tg and Con mice differ in responses to dietary fat, mice were fed a diabetogenic 60% HFD or regular chow diet (RD) for 10 weeks. Both Syn4 Tg and Con mice showed equivalently increased body weight on either RD or HFD (FIG. 20A). The relative fat pad weights of HFD-fed Tg and Con mice were similarly elevated above that of RD-fed mice (0.024 g/g BW for RD-Con and -Tg, n=4/group) (FIG. 20B), as was their food intake (FIG. 20C). Despite the obesity, HFD-fed Syn4 Tg mice retained the normal 2-fold increase in insulin-stimulated GLUT4 accumulation in skeletal muscle t-tubules/sarcolemma reported for similarly aged chow-fed control mice (dashed line) (Spurlin et al., 2004); by contrast, HFD-fed Con mice failed to respond to insulin (FIG. 20D).

Within just 2 weeks on the HFD, fasting serum insulin levels of the Syn4 Tg mice were significantly higher than those of HFD-fed Con mice (FIG. 20E), elevated relative to those reported for similarly aged chow-fed mice (Spurlin et al., 2004). Islets from HFD-fed Syn4 Tg mice also retained the 16-fold increase in glucose-stimulated insulin secretion seen in the RD-fed Con mice (dashed line), while the response of the Con-HFD islets ex vivo fell by 50% (FIG. 20F). In terms of islet insulin content, HFD-fed Syn4 Tg mice showed an increase relative to RD-fed Tg mice, whereas HFD-Con and RD-Con showed no differences (FIG. 20G). β cell area was similar between the HFD-Con and HFD-Syn4 Tg mice (FIG. 20H), with both being increased by nearly 2-fold relative to age-matched RD-fed mice (Oh et al., 2014), and rising to levels otherwise seen in the old aged mice of FIG. 19G. These data show that Syn4 enrichment preserves activity in skeletal muscle and islet β cells with aging and with adiposity.

This is the first report of an exocytosis factor promoting extension of lifespan. How Syn4 promotes healthspan is indicated by data showing that Syn4 mice exhibit accelerated and enhanced capacity for glucose uptake and insulin release, as well as faster ‘re-equilibration’ of glycemia and insulinemia following a glucose challenge. When challenged with HFD-induced obesity, skeletal muscle GLUT4 and islet insulin granule exocytosis processes were fully protected in the Syn4 Tg mice, matching levels equivalent to those of chow-fed Con mice; HFD-Con mice showed significant lapses in both exocytotic processes.

Migration inhibition factor knockout mouse (MIF KO) showed a 15% extension of lifespan (Harper et al., 2010), and MIF deficiency is linked to protection from age-related insulin resistance (Verschuren et al., 2009) and from inflammation in islets associated with type 1 and type 2 diabetes (Stojanovic et al., 2012; Stosic-Grujicic et al., 2008). Despite the similarities with MIF deficiency in terms of lifespan extension and improvements in insulin secretion and insulin action, the Syn4-enriched Tg mice did not exhibit differences in inflammation, as assessed via serum levels of inflammatory factors TNFα or IL-6, with aging or under HFD-induced inflammatory conditions (Tables 4 and 5). However, finding that the capacity of islets of HFD-fed Syn4 mice to increase insulin content, without increasing β cell mass, shows that this exocytotic protein plays an otherwise unappreciated role in insulin biosynthesis.

TABLE 4
Serum metabolite levels in aged Control and Syn4 Tg mice TNF

| | | | Cholesterol | |
|---|---|---|---|---|
| | TNF-α (pg/ml) | IL-6 (pg/ml) | (mg/dl) | FFA (mmol/l) |
| Con | 1.6 ± 0.5 | 14.6 ± 4.8 | 122.7 ± 5.7 | 1.35 ± 0.1 |
| Syn4 Tg | 1.3 ± 0.8 | 8.3 ± 3.1 | 109.6 ± 5.8 | 1.52 ± 0.1 |

Data represent the mean ± SE of 7 Con and 7 Tg mice sera at 18 months of age (combined sexes). Con, control.

TABLE 5
Serum metabolite levels in HFD-fed Control and Syn4 Tg mice

| | Con | | Syn5 Tg | |
|---|---|---|---|---|
| Weeks on HFD | 5 | 10 | 5 | 10 |
| TNF-α (pg/ml) | 3.3 ± 0.1 | 3.6 ± 0.4 | 2.9 ± 0.1 | 4.2 ± 1.0 |
| IL-6 (pg/ml) | 7.9 ± 0.4 | 12.8 ± 2.6 | 8.5 ± 1.0 | 14.4 ± 1.5 |

Data represent the mean ± SE of 3-6 Con and Tg male mice sera during the HFD study.
Con, control.

Syn4 upregulation and improvement of function in islets was not associated with substantial changes in expression of genes affiliated with lifespan extension (Table 3). However, relative phosphorylated FoxO1 in pancreata from the Syn4 Tg mice was reduced, consistent with the association of health and longevity with decreased pFoxO1 in liver (Laurent et al., 2008; Tomobe et al., 2013). Relatedly, pFoxO1 is elevated in cardiac tissue of diabetic and HFD-fed mice (Battiprolu et al., 2012). The finding of ‘normal’ insulin sensitivity in aged Syn4 Tg mice shows an increased healthspan in addition to longevity.

Preservation of insulin sensitivity into old age reflects enhanced responsiveness by the skeletal muscle to the insulin signal, itself a consequence of more rapid GLUT4 vesicle accumulation at the t-tubule/sarcolemmal membranes, leading to faster clearance of circulating glucose. This is consistent with reports citing hyperglycemia as damaging to cells and leading to shorter lifespan (Fitzenberger et al., 2013; Yang et al., 2005). Additionally, this enhanced muscle response to insulin was maintained in obese Syn4 Tg mice. The decreased serum triglyceride content of the aged Syn4 Tg mice was unexpected, particularly given that 3-6 month old Syn4 Tg mice or HFD-fed Syn4 Tg mice failed to show differences from paired Con mice in triglyceride contents (Spurlin et al., 2004).

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiments disclosed herein contemplated for carrying out this invention, but that the invention will include all embodiments falling within the scope of the claims.

What is claimed is:

1. A method for improving whole body glucose homeostasis in a subject, comprising inducing overexpression of at least one gene in the subject, wherein the at least one gene is selected from the group consisting of: Syntaxin 4; and Doc2b, thereby improving whole body glucose homeostasis.

2. The method of claim 1, wherein insulin sensitivity is improved, insulin secretion is improved, or a combination thereof in the subject, wherein improvement is relative to the insulin sensitivity or insulin secretion of the subject prior to inducing overexpression of the at least one gene in the subject.

3. The method of claim 1, further comprising inducing overexpression of the Munc18c gene in the subject.

4. The method of claim 1, wherein the subject is selected from the group consisting of: human; canine; rodent; primate; swine; equine; sheep; and feline.

5. The method of claim 1, wherein the subject is human.

6. The method of claim 1, wherein the subject suffers from an insulin-related disease or condition selected from the group consisting of: type 1 diabetes; type 2 diabetes; chronic pancreatitis; pancreatectomy; insulin resistance; prediabetes; and age-related insulin resistance.

7. The method of claim 1, wherein overexpression of the at least one gene in the subject is induced by at least one method selected from the group consisting of: cell therapy; and gene therapy.

8. The method of claim 7, wherein the cell therapy comprises administering to the subject cells overexpressing the at least one gene.

9. The method of claim 8, wherein overexpression of the at least one gene in the cells is achieved by introducing into the cells at least one nucleic acid having a sequence identity at least 80% identical to at least one nucleic acid selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; and SEQ ID NO: 8.

10. The method of claim 9, wherein the at least nucleic acid has a sequence identity selected from the group consisting of: at least 80%; at least 85%; at least 90%; at least 95%; at least 96%; at least 97%; at least 98%; at least 99%; and 100% identical with the at least one selected nucleic acid.

11. The method of claim 8, wherein the introduction of the at least one nucleic acid is by transduction via viral vector.

12. The method of claim 11, wherein the viral vector is selected from the group of viral vectors consisting of: a retroviral vector; adenovirus; herpes simplex virus; lentivirus; poxvirus; adeno-associated virus; and recombinant adeno-associated virus (rAAV).

13. The method of claim 11, wherein the viral vector is recombinant adeno-associated virus (rAAV).

14. The method of claim 9, wherein the at least one nucleic acid is operably linked to a promoter.

15. The method of claim 8, wherein the cells are autogenic, allogenic, or xenogenic.

16. The method of claim 8, wherein the cells are selected from the group of cells consisting of: β cells; differentiated stem cells; undifferentiated stem cells; precursor cells, and reprogrammed insulin producing cells.

17. The method of claim 8, wherein the cells are targeted to a particular tissue selected from the group consisting of: pancreatic tissue; skeletal muscle tissue; adipose tissue; brain tissue; heart tissue; liver tissue; spleen tissue; kidney tissue; and lung tissue.

18. The method of claim 8, wherein the cells are administered by a method of administration selected from the group consisting of: intravenous administration; and transplantation.

19. The method of claim 18, wherein cells are transplanted in at least one tissue selected from the group consisting of: pancreatic tissue; skeletal muscle tissue; adipose tissue; brain tissue; heart tissue; liver tissue; spleen tissue; kidney tissue; and lung tissue.

20. The method of claim 8, wherein the cells are β cells overexpressing Syntaxin 4, and wherein the cells are transplanted in a tissue of the subject.

21. The method of claim 20, wherein the tissue is kidney tissue.

22. The method of claim 8, wherein the cells are autologous β cells isolated from the subject, transduced to induce overexpression of Syntaxin 4, and transplanted back into the subject.

23. The method of claim 7, wherein the gene therapy comprises administering to the subject at least one nucleic acid associated with the at least one gene.

24. The method of claim 23, wherein overexpression of the at least one gene is achieved by administering to the subject at least one nucleic acid having a sequence identity at least 80% identical to at least one nucleic acid selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; and SEQ ID NO: 8.

25. The method of claim 24, wherein the at least one nucleic acid has a sequence identity selected from the group consisting of: at least 80%; at least 85%; at least 90%; at least 95%; at least 96%; at least 97%; at least 98%; at least 99%; and 100% identical with the at least one selected nucleic acid.

26. The method of claim 23, wherein the at least one nucleic acid is present as a naked DNA, in a plasmid, or in a vector.

27. The method of claim 26, wherein the vector is selected from a group of vectors consisting of: a retroviral vector; adenovirus; herpes simplex virus; lentivirus; poxvirus; adeno-associated virus; and recombinant adeno-associated virus (rAAV).

28. The method of claim 23, wherein the at least one nucleic acid is operably linked to a promoter.

29. The method of claim 28, wherein the promoter is tissue specific.

30. The method of claim 28, wherein the promoter is specific for a tissue selected from the group consisting of: pancreatic tissue; skeletal muscle tissue; adipose tissue; brain tissue; heart tissue; liver tissue; spleen tissue; kidney tissue; and lung tissue.

31. The method of claim 28, wherein the promoter is β cell specific.

32. The method of claim 23, wherein the at least one nucleic acid is administered to the subject by at least one method selected from the group consisting of: intravenous injection; direct organ or tissue injection; organ surface instillation; intra-arterial injection; intraportal injection; and retrograde intravenous injection.

33. The method of claim 32, further comprising at least one physical method to enhance delivery of the nucleic acid selected from the group consisting of: electroporation; sonoporation; mechanical massage; and ultrasound exposure.

34. The method of claim 33, wherein the at least one physical method is applied to a target tissue.

35. The method of claim 34, wherein the target tissue is selected from the group consisting of: pancreatic tissue; skeletal muscle tissue; adipose tissue; brain tissue; heart tissue; liver tissue; spleen tissue; kidney tissue; and lung tissue.

36. The method of claim 23, wherein the at least one nucleic acid is chemically modified.

37. The method of claim 36, wherein the chemical modification is selected from the group consisting of: lipoplex condensation and encapsulation; polymersome condensation and encapsulation; polyplex complex formation; dendrimer complex formation; inorganic nanoparticle complex formation; and cell penetrating peptide complex formation.

38. The method of claim 37, wherein the chemical modification further comprises the addition of a tissue specific peptide.

39. A pharmaceutical composition comprising at least one nucleic acid associated with a gene selected from the group consisting of: Syntaxin 4; and Doc2b, wherein the at least one nucleic acid is operably linked to a promoter.

40. The pharmaceutical composition of claim 39, wherein the composition further comprises a nucleic acid encoding Munc18c, wherein the nucleic acid encoding Munc18c is operably linked to a promoter.

41. The pharmaceutical composition of claim 39, wherein the at least one nucleic acid shares a sequence identity of at least 80% with at least one nucleic acid selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; and SEQ ID NO: 8.

42. The pharmaceutical composition of claim 41, wherein the at least one nucleic acid shares a sequence identity selected from the group consisting of: at least 80%; at least 85%; at least 90%; at least 95%; at least 96%; at least 97%; at least 98%; at least 99%; and 100% with the at least one selected nucleic acid.

43. The pharmaceutical composition of claim 39, wherein the at least one nucleic acid is present in a vector.

44. The pharmaceutical composition of claim 43, wherein the vector is selected from the group consisting of a retroviral vector; adenovirus; herpes simplex virus; lentivirus; poxvirus; adeno-associated virus; recombinant adeno-associated virus (rAAV); and naked plasmid DNA.

45. The pharmaceutical composition of claim 39, further comprising a pharmaceutically acceptable excipient.

46. The pharmaceutical composition of claim 39, wherein the promoter is specific for a tissue selected from the group consisting of: pancreatic tissue; skeletal muscle tissue; adipose tissue; brain tissue; heart tissue; liver tissue; spleen tissue; kidney tissue; and lung tissue.

47. A method for improving the healthspan of a subject, comprising inducing overexpression of Syntaxin 4 in the subject, thereby improving healthspan.

48. The method of claim 47, wherein the subject is selected from the group consisting of: mouse; and human.

49. The method of claim 47, wherein the subject is of an advanced age, obese, or a combination thereof.

50. The method of claim 47, wherein insulin sensitivity, insulin secretion, or a combination thereof is improved or conserved in the subject.

51. The method of claim 47, wherein the lifespan of the subject is extended.

52. The method of claim 47, wherein the overexpression of Syntaxin 4 occurs in at least one cell type selected from the group consisting of: skeletal muscle cells

53. The method of claim 47, wherein the subject suffers from an insulin related disease or condition selected from the group consisting of: type 1 diabetes; type 2 diabetes; chronic pancreatitis; pancreatectomy; insulin resistance; prediabetes; and age-related insulin resistance.

54. The method of claim 47, wherein overexpression is induced by at least one method selected from the group consisting of: cell therapy; and gene therapy.


SEQUENCE LISTING (13 sequences)

SEQ ID NO: 1 - DNA, 894 bp, Homo sapiens
atgcgcgacaggacccacgagctgagacagggggatgacagctcggacgaagaggacaaggagcgggtcgcgctggtggtgcacccgggcacggcacggctggggagcccggacgaggagttcttccacaaggtccggacaattcggcagactattgtcaaactggggaataaagtccaggagttggagaaacagcaggtcaccatcctggccacgccccttcccgaggagagcatgaagcaggagctgcagaacctgcgcgatgagatcaaacagctggggagggagatccgcctgcagctgaaggccatagagccccagaaggaggaagctgatgagaactataactccgtcaacacaagaatgagaaaaacccagcatggggtcctgtcccagcaattcgtggagctcatcaacaagtgcaattcaatgcagtccgaataccgggagaagaacgtggagcggattcggaggcagctgaagatcaccaatgctgggatggtgtctgatgaggagttggagcagatgctggacagtgggcaaagcgaggtgtttgtgtccaatatcctgaaggacacgcaggtgactcgacaggccttaaatgagatctcggcccggcacagtgagatccagcagcttgaacgcagtattcgtgagctgcacgacatattcacttttctggctaccgaagtggagatgcagggggagatgatcaatcggattgagaagaacatcctgagctcagcggactacgtggaacgtgggcaggagcacgtcaagacggccctggagaaccagaagaaggcgaggaagaagaaagtcttgattgccatctgtgtgtccatcaccgtcgtcctcctagcagtcatcattggcgtcacagtggttggataa

SEQ ID NO: 2 - DNA, 1372 bp, Mus musculus
gcaggatggggggaagagaagggaattccaacctgtagaactttgggggtcctatgatcgggttcttccccacgactgtggatggtgaaaggggtggggcctttggcggctccggggtgtactggagttcgctgggtgagggagggggtgtcaagtgtgagagaggtgcaggacttccaaatttagaaccttgaggccctttgggctcgggtccagggcactccgaccaccatgcgcgacaggacccacgagttgaggcagggggataacatctcagacgatgaagatgaggttcgagtcgcgctggtggtgcactcaggtgctgcccggttgggcagcccggacgacgagttcttccagaaggtgcagacaattcggcagactatggccaaactggagagtaaagtccgggagttggagaaacagcaggtcaccatcctggccacgcctcttcccgaggagagcatgaagcagggcctgcagaacctgcgagaggagatcaaacagctggggagagaggtccgggcgcagctaaaagccatagagccccagaaggaggaagctgatgagaattacaactcagtcaacacaaggatgaagaaaacccagcatggagtcctgtcccagcaatttgtcgagctcatcaacaagtgcaactcaatgcagtccgaataccgagagaagaatgtggagcggatccggaggcagctgaagatcaccaatgctggaatggtgtctgacgaggagctggaacagatgctggacagtgggcagagtgaggtgtttgtgtctaatatcctgaaggacacgcaggtgactcggcaggccctgaatgagatctctgcgcggcacagtgagatccagcagttggagcgcagtatccgagagctccatgagatctttacgtttctagctacggaggtggagatgcagggggagatgatcaaccgcatcgagaagaacatcctgagctcggccgactacgtggaacgtgggcaagagcacgtcaagatagccctagagaatcagaagaaggcgaggaagaaaaaagtcatgattgccatctgtgtctctgtcactgttctcatcttggctgtcatcattggcatcaccataaccgttggataatgtcacatgttcttggcattgggagagacagagacccagcttttgtggaggtggggcaggcctccaactgaacccgtccgcacactggaccttgtgcagagggcctgcggctcgtcctgggttggcagctgctcattcatggagtcctcccctggagtcctccacaatgcctgcgagacagttttcaatgtcctgtttgtctgggatacatggttttgtaaaaaatttaaaaaaaaaaaaaaaa

SEQ ID NO: 3 - DNA, 894 bp, Homo sapiens
  modified_base (519)..(519): a, c, t or g
atgcgcgacaggacccacgagctgagacagggggatgacagctcggacgaagaggacaaggagcgggtcgcgctggtggtgcacccgggcacggcacggctggggagcccggacgaggagttcttccacaaggtccggacaattcggcagactattgtcaaactggggaataaagtccaggagttggagaaacagcaggtcaccatcctggccacgccccttcccgaggagagcatgaagcaggagctgcagaacctgcgcgatgagatcaaacagctggggagggagatccgcctgcagctgaaggccatagagccccagaaggaggaagctgatgagaactataactccgtcaacacaagaatgagaaaaacccagcatggggtcctgtcccagcaattcgtggagctcatcaacaagtgcaattcaatgcagtccgaataccgggagaagaacgtggagcggattcggaggcagctgaagatcaccaatgctgggatggtgtctgatgaggaggcngcncagatgctggacagtgggcaaagcgaggtgtttgtgtccaatatcctgaaggacacgcaggtgactcgacaggccttaaatgagatctcggcccggcacagtgagatccagcagcttgaacgcagtattcgtgagctgcacgacatattcacttttctggctaccgaagtggagatgcagggggagatgatcaatcggattgagaagaacatcctgagctcagcggactacgtggaacgtgggcaggagcacgtcaagacggccctggagaaccagaagaaggcgaggaagaagaaagtcttgattgccatctgtgtgtccatcaccgtcgtcctcctagcagtcatcattggcgtcacagtggttggataa

SEQ ID NO: 4 - DNA, 894 bp, Mus musculus; identical to SEQ ID NO: 3
  modified_base (519)..(519): a, c, t or g

SEQ ID NO: 5 - DNA, 2026 bp, Homo sapiens
gcggagcgggcagcggccaagtcagggccgtccgggggcgcggccggcgatgcccgcagcccccgccgcgccccgccgggcctgctgagccgcccccgggccggggtcgcgccgggccgggccgcgcccggggcggggcggcgctgcctgcatgaccctccggcggcgcggggagaaggcgaccatcagcatccaggagcatatggccatcgacgtgtgccccggccccatccgtcccatcaagcagatctccgactacttcccccgcttcccgcggggcctgcccccggacgccgggccccgagccgctgcacccccggacgcccccgcgcgcccggctgtggccggtgccggccgccgcagcccctccgacggcgcccgcgaggacgacgaggatgtggaccagctcttcggagcctacggctccagcccgggccccagcccgggtcccagccccgcgcggccgccagccaagccgccggaggacgagccggacgccgacggctacgagtcggacgactgcactgccctgggcacgctggacttcagcctgctgtatgaccaggagaacaacgccctccactgcaccatcaccaaggccaagggcctgaagccaatggaccacaatgggctggcagacccctacgtcaagctgcacctgctgccaggagccagtaaggcaaataagctcagaacaaaaactctccgtaacactctgaaccccacatggaacgagaccctcacttactacgggatcacagatgaagacatgatccgcaagaccctgcggatctctgtgtgtgacgaggacaaattccggcacaatgagttcatcggggagacacgtgtgcccctgaagaagctgaaacccaaccacaccaagaccttcagcatctgcctggagaagcagctgccggtggacaagactgaagacaagtccctggaggagcggggccgcatcctcatctccctcaagtacagctcacagaagcaaggcctgctggtaggcatcgtgcggtgcgcccacctggccgccatggacgccaacggctactcggacccctacgtgaaaacatacctgaggccagatgtggacaagaaatccaaacataagacagcggtgaagaaaaaaaccctgaacccggagtttaatgaggagttctgttacgagatcaagcatggggacctggccaagaagtccctggaggtcaccgtttgggattacgacattggaaaatccaacgatttcattggtggtgtggttctgggcatccacgccaagggggagcgcctgaagcactggtttgactgcctgaagaacaaggacaagcgcatcgagcgctggcacacgctcaccagcgagctcccaggggctgtgctcagcgactgacgcccacccgccactgctacccctgccgccacctgcgcccagcacggccggccccgggcttccccagcagccaccaaggcctgtggcccccacactgggggagatccagaacccctgcttggacacagagccactgcagtccccgctcggaggatgtggagggctcagccactctgggacggggagggcaaggagctggggtggggggctctcagctctctggggcccaagaggccggtggtggaaagagacctcagcacctgcccaggggaaggggacacgcccatctgggagcaaagacccttctagaggccagccccggctgagaggacaggagtgtgggggcgccttggcggacagtgggaacagaggagggaggtggtgagcagacagacaggtggaggatgggaccttgaagactggctgctccagcccaagaaagcctaactgcatccctcatctccttcgctgctggacagatggaagaagcgggcctgccggccgaaagtctgccagagttcccggaggctcctgatgatgggtaaattggcacatgcttcactcaatgattccacaagccctgggggtgaatgagacacagggcctgccctcagggagttcccatctagtcaggaa

SEQ ID NO: 6 - DNA, 4275 bp, Mus musculus
ctcgcaccggccgccgccgagcaggcagcagccgcgtcagggccgtccgggggccacgctggcgatgaccgcagcccccgcagcgccccgcgggacccgctgacttgcccccgggccggggtcacaccgggccgggccgggccgcgcgccgcggcgctgcctgcatgaccctccggcggcgcggggagaaggcgaccatcagcatccaggagcatatggccatcgacgtgtgtcccggccccattcggcctatcaagcagatctccgattattttccccgcttcccgcggggcctcccccctaccgccgcgccccgcgcccccgcgcccccggacgcccccgcgcgctctcccgcagccagcgccagcccccgcagcccctccgacggcgcccgcgacgacgacgaagatgtggaccagctcttcggagcctacggagccagcccaggccccagccccggccccagccccgcgaggccgcccgccaagccccccgaggacgagccggacgtcgacggctacgagtcagacgactgcaccgccctgggtacgctggacttcagtctgctctatgaccaggagaacaacgcactgcactgcaccatcagcaaggccaagggcctgaagccgatggaccacaatggactggctgatccctacgtcaaactacacctgctgcctggagccagcaaggcaaataagctcagaacaaaaactcttcggaacaccctgaacccctcgtggaacgagaccctcacttattacggaatcacggatgaggacatggtccgaaagaccctgaggatctccgtgtgtgatgaggacaaattccgccacaatgagttcattggagagactcgggtgcccctgaagaagctgaagcccaatcacaccaagacattcagcatctgcctggagaagcagctgccggtggacaaggcagaggacaagtctctggaagagcgaggccgcatcctcatctccctcaagtacagctcacagaagcagggcctgctggtgggcatcgtgcgctgtgcacacctggctgccatggatgctaatggctactcggacccctatgtgaaaacatatctgaagccagatgtagacaagaaatccaagcataagacagcagtgaagaagaaaacactaaacccagaattcaatgaggaattctgttacgagatcaagcatggagacctggccaaaaagactctggaggtcactgtctgggattatgacattggaaaatccaatgatttcatcggtggtgtggttctgggcatcaacgccaagggcgagcgcctgaagcactggtttgactgcttgaagaacaaggacaagaggattgagcgttggcacacgctcaccaatgagctcccaggggctgtactcagcgactgactgtcccatctgctgccacccacccttgccacccggcccacacaggtccaaccctgggctttctcagctgccgccaagggcaagatccaagttgtctgctcggacatagacagtgcagcccctgctaggaggccaagagcacccagcctccctcagagggacaggaaaacacaacatgatacctgttccagctccctgggcatccaagctggccagagctgggggaatcctcagcctcagctccatccagaggaagggctatctacagtgaggacctattggaggtgaggggtggagccctgctgcaagcccagaaatgggggtactctggctgcctggagactacagggaggacaatgggcaggcagagcccaggactccaggacagacaatgtagccagccagagcttccaacttgcttgtgcattgatgggcagatagactccgtgagcctctgggacggggattcccactctagcagggcatctgggtgcacatattgctacaaaggggtgcagcataccagaaccagccagttgtctgcaggggctgtgggtactgctgactgctgaagggtggagggcaaggaaggcatcccatgagagtcagggctcagcacagtcttgaaaacccagctgtggcttgaaagctaggagaaaaccaaatacagaaaagaggaggcctgaaagagggtaccaggggacagggagccatgggggcctagggttgcgctagggcagcattgggcttttaggcaggagctaggcttagggatgtgtacaagcacttgggaaacaatgttcaaacccaagctctcccatttgatcttgctcccaaggggtctcaatgcacactgctcctgttctcagcgtaaattcccagtcagcttcaggggaatgagaaagggatcctactaatgtggaaagaagcaagcatccctcctgcccacctgtccctcccttcttcatcccctcagctcaagctggtgttaggtccttcacggctacctatggcatcctggccctatcaagacagtcaggaccagcagtggaaaatagaaagccttgtttgggggaaggtggagggatgggggttctgccctcaggagctgagaactctggcctcagtttctaggaaagggtaggacactggtcagtcttgaagctcccaactgcctcctgccaactgggattcaggagcacagggggaacagggaaccaaggagttccaccatcatctcagagccccattttcctcaatccatcactgagtgataagacactggggtcctgccctaagcctagaaccctcctacagctgaaataagaactcggggtcccagaacatgattccaatccacagtgctcaccagccttccctagttggcagggaaaataggtctctagctctccacagcaccaagccacctgttgcttgtaagagactgggtttccctctttaaaaggtgtcgcctgttactgaattggcaaacgccttcttattgaatcttcaccatcctgtgtggtgggaggcatggtcaacccattttataaatgagatggctccagagttgttgataaacttccagggccaccacggtgtgaagggctgaagcaaaaactccaagccttttgactatctgaggacaaggactctcaggtccctctctccggagttaatggggcttagtgaggtctcttacagagaagtcttgttattgtcagccacctcaccctctagctggctttgaaatcatttgtgccatttaggatttgccaaaggctacagccaagccctggaactcagctgtcccacccccagcctgaggtcaaggaacttgggggaccggcctgatgttttccccatacagctagaaacaggagcagatatttaagtgcacctggttgagctctgagtaactcagattttgccttttggggtgctttgttcagagaggagtttagaaaccccccgacgatacagaaatgtttattatacctaaatgtttattatacctgaggtgactgaagctcatcaaaagaacaggtggctcaggtggacaagaaacctggatccctgttgcaccaggcagtaagtgggtttgggactcaatgatgatccttgccaatcccagggacaggcagggtgggaagcagggcttctagccacctaaaaagtgatggccactaaaaagtgatggaactttcccggatcctggaacgtttctaaatccccagtacattacatctctactaaagggaaaaggtcctttgagaagtttcagaatcagttcagtctttcattccaaacgagtatcttccttctggctcatcagcgttgcaaagaggaaatttgataccaaaaacaaaggtcattccaaagtactgtcagtaaccagggcctctcttgatgcccatcatccacctcgcgtcctggtctctgccccgccctccaccaccgttagccaatccctgaacagtcccgtcttccaacccagagtcagtgacctcctgacctctagtggcaaaaatgaggaactgcacctccgtggcccgcccgcccgcccgcccgcccgccgagctcgggccaagcgtcctgccctccgtccttctccagccaagtgaatctcccgatgcatggactctggctgtcctcgacacggactcgtgcactgcttacagttttgctgccacgtgacgactgcagaatacatcctcaaactgcagcgccacgaaacaatttttgtaatcttgtctgaccttcccaaaggtgtcctttttaagtcttatttgttaatatttataacgatatataatcaaagtaaatgagaacgtcgtactgaaaaaaaaaaaaaaaaaaaaaaaaaaaaaaa

SEQ ID NO: 7 - DNA, 2522 bp, Homo sapiens
accccaacgccgcttctgcggccaaagtaggttgggagtggaaggtggtggctgctgctccgcagtgtcgggaagatggcgccgccggtggcagagagggggctaaagagcgtcgtgtggcagaagataaaagcaacagtgtttgatgactgcaagaaagaaggcgaatggaagataatgcttttagatgaatttaccactaagcttttggcatcgtgttgcaaaatgacagatcttctagaagaaggtattactgttgtagagaatatttataagaaccgtgaacctgtcagacaaatgaaagctctttatttcatcactccgacatcaaagtctgtagattgtttcttacatgattttgcaagtaaatcggagaacaagtataaagcagcatatatttacttcactgacttttgccctgataatctctttaacaaaattaaggcttcttgctccaagtcaataagaagatgtaaagaaataaatatttccttcattccacatgaatctcaggtgtatactcttgatgtaccagatgcattctattactgttatagtccagaccctggtaatgcaaagggaaaagatgccattatggaaacaatggctgaccagatagttacagtgtgtgccaccttggatgaaaatcccggagtaagatataaaagtaaacctctagataatgccagtaagcttgcacagcttgttgaaaaaaagcttgaagactactacaagattgatgaaaagagcctaataaagggtaaaactcattcacagctcttaataattgatcgtggctttgatcctgtgtccactgtcctgcatgaactgacctttcaggcaatggcatatgatctactaccaattgagaatgatacatacaaatataaaacagatggaaaagaaaaggaggccatccttgaagaagaagatgacctctgggttagaattcgacatcgacatattgcggttgtgttagaggaaattcccaagcttatgaaagaaatttcatcaacaaagaaagcaacagaaggaaagacatcacttagtgctcttacccagctgatgaaaaagatgccccatttccgaaaacagattactaagcaagttgtccatcttaacttagcagaagattgcatgaataagttcaagcttaatatagaaaagctctgcaaaactgaacaggacctggcacttggaactgatgcagaaggacagaaggtgaaagattccatgcgagtactccttccagttctactcaacaaaaatcatgataattgtgataaaataagagcaattctactttatatcttcagtattaatggaactacggaagaaaatttggacaggttgatccagaatgtaaagatagaaaatgagagtgacatgattcgtaactggagttaccttggtgttcccattgttccccaatctcaacaaggcaaaccgttaagaaaggatcggtctgcagaagaaacttttcagctctctcggtggacaccttttatcaaagatattatggaggatgctattgataatagattagattcaaaagaatggccatattgttcccagtgtccagcagtatggaatggttcaggagctgtaagtgctcgccagaaacccagagctaattatttagaagaccgaaaaaatgggtcaaagctgattgtttttgtaattggagggatcacatactctgaagtgcgttgtgcttatgaagtttctcaggcacataaatcctgtgaagttattattggttctacacatgttttaacacccaaaaagctgttggatgatataaagatgctgaataaacccaaggataaagtctccttaattaaagatgaatagcatttctttttggagggtttagagattcttactaatatgttgaactaaaatagaaagaaaatgttgctgtcatgtaatttaaacaatgtaaatattttatggaataatggcttttcaaatacatttcttaaggaactgtttatgattattactggatttgtcatttttgataatttaaatattgctgctgctttgtagatgatgagaagaaatgttaaagtgctttctaaaaggaaattttttcacctttggaggagaatatattagagttgtgggtaatttttcacagccacctatgtacatactaattacccattggatacttatatctaaaagtctcatgctgaagtatagtttttgggaaagaatgattttaaataaagagattgtaaaagtaaaaaactgtaaatgtatatgtatgatagaattgtttcctctaagtgtagtttttctttcaactaaaattcagtttatgtgtaaaataattcagtcattaatagaaatggagtgatttcacagtgtgtactgttttgccacatacttctaaagaacacaattttatataattttgaaatcatgtatgtttaaattagaaaaccaaaaatcatgaacattctaagagaaaataaatatagaatttaaaaaattaaaaaaaaaaaaaaaaaaaa

SEQ ID NO: 8 - DNA, 2406 bp, Mus musculus
gaggcggtggctgtagctccacggctgggggaagatggcgccgccggtatcggagcggggactgaagagcgtcgtgtggcggaagataaaaacagccgtgttcgatgactgccggaaagaaggcgaatggaagataatgctgttagatgagtttaccaccaaacttttgtcgtcatgctgcaaaatgacagaccttctagaggagggcataactgttatagagaatatttataagaatcgtgaacctgtcagacaaatgaaagctctttatttcatctctccaacaccaaaatctgtagattgtttcttgagagattttggaagtaaatctgagaaaaaatacaaagcagcgtatatatacttcactgacttttgtcctgacagtctctttaacaagattaaagcatcttgctccaagtcaataagaagatgtaaggaaataaacatttccttcattccacaggaatctcaggtttatactcttgatgtaccggacgcattctattactgttacagtccagaccctagtaacgccagcaggaaagaagtggtcatggaggcaatggctgagcagattgtgacagtgtgtgccactctggatgaaaaccctggagtgaggtacaagagtaaacctctagataatgccagtaagcttgcacagctggttgagaaaaagcttgaagactactacaaaattgatgaaaaaggcctaataaagggtaaaactcagtcccagctcttaataattgaccgtggctttgaccctgtgtccactgtcctgcatgaactgacctttcaggcaatggcatatgatctactaccaattgagaatgatacatacaagtacaaaacagatggaaaagagaaggaggcagttcttgaagaagacgacgacctgtgggtgcgggttcgacaccggcacatcgcggttgtgttggaggaaattccaaagcttatgaaggaaatttcatcaacaaagaaagctacagaggggaagacatcacttagcgctcttacccagctgatgaaaaagatgccgcacttccgaaagcagatctcgaagcaagtagtccatcttaacttagctgaagactgcatgaataagtttaagctgaatattgagaagctctgcaaaactgagcaggacctggcacttggaacagatgctgaaggccagcgggtgaaggactccatgctggtgctcctcccagtgctgctcaacaaaaaccatgacaactgcgacaaaatacgggcagtcctgctctacatcttcgggattaatggaaccactgaagaaaatctggacagactgatccacaatgtaaagatagaagatgatagtgatatgattcgtaactggagccaccttggtgttcccattgttcccccatcccagcaagccaaaccactgagaaaggatcggtctgcagaagagacttttcagctttctcgatggacaccttttatcaaagatatcatggaggatgccattgataatagattagattccaaagagtggccgtattgttcccggtgcccagcagtgtggaatggctctggagctgtgagtgctcgccagaaacccagaactaactacttagagctggaccggaaaaatgggtcaaggctgattatttttgtaattggaggaattacgtactctgagatgcggtgtgcttatgaagtttcccaggcacataaatcctgtgaggttattattggttccacacatattttaacacccagaaagctcctggatgatataaaaatgctgaataaatcaaaggataaagtttcctttaaggatgagtaactttttatggttgtcgtttagaggttttgttaatacgatcggctacagcaaatgttgctgttgtaatttaagcggtgtaaatagtgtatggagtaatgtggttttcaagtgtacatcttacagaagcttatgtgttatggatttgccatttctaataatttaaatactgttgctgctttgtacaagatctgacactacagggctttctaatcaggatgtcttcaccctagggacagaatctgttaggagggatgggtagtttttacagcctgctgtatacatactagttactgtatacttacgctgaaatatagtgttgggggcaaaatgattttgaatgaagagattataaaagtaaaaactgtaaatgtatgtatgatagaattatttcttataagtgcagtttcttaaaactgagattcataattttttatgtgtaaaataattccagtgtttaatatgatttcactgtgtatattacctgtgaaataattctaaagaacacaattttatatgtttgagatcatgtctgcttaagttagacgccaaaaataatcatgaacattctaagagaaaataaatatattattgtc

SEQ ID NO: 9 - DNA, 20 bp, Artificial Sequence
  Description of Artificial Sequence Synthetic primer
ggcagaggacaagtccctgg

SEQ ID NO: 10 - DNA, 20 bp, Artificial Sequence
  Description of Artificial Sequence Synthetic primer
agaggattgagcgttggcac

SEQ ID NO: 11 - DNA, 24 bp, Artificial Sequence
  Description of Artificial Sequence Synthetic primer
acgacgaggcttgcaggatcataa

SEQ ID NO: 12 - DNA, 20 bp, Artificial Sequence
  Description of Artificial Sequence Synthetic primer
ggaaagaaggcgaatggaag

SEQ ID NO: 13 - DNA, 20 bp, Artificial Sequence
  Description of Artificial Sequence Synthetic primer
tcactccagggttttcatcc